SEQUENCE LISTING (19 sequences)

SEQ ID NO: 1 - DNA, 1986 bp, Mus musculus
  misc_feature (1494)..(1496): n is a, t, c or g
tcaagaatatgctttgccttattgcctgtgactttctgagattcaattatagtatctgttaaattctaatgttaaagagaactcttttttccgctttgtgtaagttaacctatattgattaccaatatcaaataaaaaggtcctgtaatgagaataatcacctttaacctcctcggcaaaacagcaaagcgtatgccatatcatagcgtgtcgcagcgcgaatttgagcaaatctacccaaaaccaggttgggtagaacacgacccaatggaaatctgggccacccaaagctccacgctggtagaagtgctggcgaaagccgatatcagttccgatcaaattgcagctatcggtattacgaaccagcgtgaaaccactattgtctgggaaaaagaaaccggcaagcctatctataacgccattgtctggcagtgccgtcgtaccgcagaaatctgcgagcatttaaaacgtgacggtttagaagattatatccgcagcaataccggtctggtgattgacccgtacttttctggcaccaaagtgaagtggatcctcgaccatgtggaaggctctcgcgagcgtgcacgtcgtggtgaattgctgtttggtacggttgatacgtggcttatctggaaaatgactcagggccgtgtccatgtgaccgattacaccaacgcctctcgtaccatgttgttcaacatccatatccctggactgggacgacaaatgctggaagtgctggatattccgcgcgagatgctgccagaagtgcgtcgttcttccgaagtatacggtcagactaacattggcggcaaaggcggcacgcgtattccaatctccgggatcgccggtgaccagcaggccgcgctgtttggtcagttgtgcgtgaaagaagggatggcgaagaacacctatggcactggctgctttatgctgatgaacactggcgagaaagcggtgaaatcagaaaacggcctgctgaccaccatcgcctgcggcccgactggcgaagtgaactatgcgttggaaggtgcggtgtttatggcaggcgcatccattcagtggctgcgcgatgaaatgaagttgattaacgacgcctacgattccgaatatttcgccaccaaagtgcaaaacaccaatggtgtgtatgtggttccggcatttaccgggctgatttaccgggctggtgcgccgtactgggacccgtatgcgcgcggggcgattttcgtctactcgtgggtgaacgctaaccacattatacagcgcatcgctgggttccaggctagctattctgtattgaatgcggaactgtgcgtgccgcaaacatcgatgcgggggccgaagggcgtgcggtggtgaatctcaaccgcttcgacctcgccatgctgaaaccgtttatgccagaaaccactcaggccagcggtatcttcacgggtaaagcggacgttgcctgggacaccacgaaagaggggctgccgcagggcagtatcaccnnnccggttttgtcgccaatgatgccgcccaccagcgcaccgagaaacattccggcggtcgtgattgctgagaatgtggctgtggtggaattatctgtccagcccaacgctttcagctgcgcgaggatcaagccaccaacggcattactccagcagacaagcaagccaaacgcgacgatggcaaacattgatgaatgccagcggcaatccggtaagcgatccagccgtgcaccacaatgcggttaagataatagctggtacccattacactggcgttttctcttcacgccagtcggtcgtgacttctgctaacaccgcagccggagattttccgttcaggcgcgtgacgcctgcttctgattgcctgctctccaggcagtggtcgccctgataaagccaggcgcgcagattggtcgatccccagtcaattgcgatgtagcgagctgtcatgtgatttcctttaaccttcgtgtcgagctggcgatcatggtaa

SEQ ID NO: 2 - protein, 403 aa, Mus musculus
MRIITFNLLGKTAKRMPYHSVSQREFEQIYPKPGWVEHDPMEIWATQSSTLVEVLAKADISSDQIAAIGITNQRETTIVWEKETGKPIYNAIVWQCRRTAEICEHLKRDGLEDYIRSNTGLVIDPYFSGTKVKWILDHVEGSRERARRGELLFGTVDTWLIWKMTQGRVHVTDYTNASRTMLFNIHIPGLGRQMLEVLDIPREMLPEVRRSSEVYGQTNIGGKGGTRIPISGIAGDQQAALFGQLCVKEGMAKNTYGTGCFMLMNTGEKAVKSENGLLTTIACGPTGEVNYALEGAVFMAGASIQWLRDEMKLINDAYDSEYFATKVQNTNGVYVVPAFTGLIYRAGAPYWDPYARGAIFVYSWVNANHIIQRIAGFQASYSVLNAELCVPQTSMRGPKGVRW

SEQ ID NO: 3 - protein, 505 aa, Pseudomonas aeruginosa
MTDKHNKKYVVALDQGTTSSRAIVFDRDANVVSQAQREFAQFYPQAGWVEHDPMEIWATQSSTLVEALAQASIEHDQVAAIGITNQRETTVVWDRHSGRPIHNAIVWQCRRSAAICAQLKRDGLEDYIRETTGLVTDPYFSGTKLKWILDNVEGARERARNGDLLFGTIDTWLIWKLTEGKVHVTDYTNASRTMLFNIHSRDWDARMLEVLDIPRSMLPEVRNSSEVYGNARIGGVGGGELPIAGIAGDQQAALFGQMCVEPGQAKNTYGTGCFLLMHTGDKAVKSTHGLLTTIACGPRGEVGYALEGAVFNGGSTVQWLRDELKVINDSFDSEYFATKVKDSNGVYLVPAFTGLGAPYWDPYARGAVFGLTRGVKADHLIRATLESIAYQTRDVLDAMQRDAGERLRALRVDGGAVANNFLMQFQADILGTRVERPVMRETTALGAAYLAGLACGFWSSLDELKSKAVIERVFEPECDEPRREKLYAGWKKAVERTRGWDDGEL

SEQ ID NO: 4 - protein, 524 aa, Mus musculus
MAAAKKAVLGPLVGAVDQGTSSTRFLVFNSKTAELLSHHQVEIKQEFPREGWVEQDPKEILQSVYECIEKTCEKLGQLNIDISNIKAIGVSNQRETTVVWDKVTGEPLYNAVVWLDLRTQSTVENLSKRIPGNNNFVKSKTGLPLSTYFSAVKLRWLLDNVKKVQEAVEENRALFGTIDSWLIWSLTGGIHGGVHCTDVTNASRTMLFNIHSLEWDKELCEFFGIPMEILPNVRSSSEIYGLMKAGALEGVPISGCLGDQSAALVGQMCFQDGQAKNTYGTGCFLLCNTGHKCVFSEHGLLTTVAYKLGRDKPVYYALEGSVAIAGAVIRWLRDNLGIIKSSEEIEKLAKEVGTSYGCYFVPAFSGLYAPYWEPSARGIICGLTQFTNKCHIAFAALEAVCFQTREILDAMNRDCGIPLSHLQVDGGMTSNKILMQLQADILYIPVVKPSMPETTALGAAMAAGAAEGVGVWSLEPEDLSAVTMERFEPQINAEESEIRYSTWKKAVMKSIGWVTTQSPESGIP

SEQ ID NO: 5 - protein, 553 aa, Homo sapiens
MAAPKTAAVGPLVGAVVQGTNSTRFLVFNSKTAELLSHHKVELTQEFPKEGWVEQDPKEILQSVYECIARTCEKLDELNIDISNIKAVGVSNQRETTVIWDKLTGEPLYNAVVWLDLRTQTTVEDLSKKIPGNSNFVKSKTGLPLSTYFSAVKLRWMLDNVRNVQKAVEEGRALFGTIDSWLIWSLTGGVNGGVHCTDVTNASRTMLFNIHSLEWDKELCDFFEIPMDLLPNVFSSSEIYGLIKTGALEGVPISGCLGDQCAALVGQMCFQEGQAKNTYGTGCFLLCNTGRKCVFSEHGLLTTVAYKLGREKPAYYALEGSVAIAGAVIRWLRDNLGIIETSGDIERLAKEVGTSYGCYFVPAFSGLYAPYWEPSARGILCGLTQFTNKCHIAFAALEAVCFQTREILEAMNRDCGIPLRHLQVDGGMTNNKVLMQLQADILHIPVIKPFMPETTALGAAMAAGAAEGVSVWSLEPQALSVLRMERFEPQIQATESEIRYATWKKAVMKSMGWVTSQSPEGGDPSIFCSLPLGFFIVSSMVMLIGARYISGVP

SEQ ID NO: 6 - DNA, 1793 bp, Mus musculus
gccgctgtacggtccggaattccgggacgacccacacgtccgggcggaccctaggtggtacaacatggcggcgctcggcacgcagcttccgtgacctgtagccgagcctcttgtgtttgtagctgagtttggttgggcgcgggcaccgccttggggcagcagccggagagggagccatggggacagtgcacgcccggagtctggagcccctcccgtcgagtggaactgactttggagcactgggggaggaagccgagtttgtggaagttgagccggaagcgaaacaggaaatcctggaaaacaaagatgtggtggttcagcatgttcattttgatggacttgggcggactaaggatgacatcatcatttgtgaaatcggagaggtctttaaggctaaaaacctcattgaggtaatgcggcgatctcatgaagcccgggaaaaactgcttcgcctaggaatttttagacaagtggatgttttgatcgatacatgtcatggtgaagatgccctgcccaatgggttagatgtcacctttgaagtgacagagctgaggagactgacgggcagttacaacaccatggttggaaacaacgaaggcagtatggtactcggcctcaaactccccaaccttctgggacgagcagaaaaagtcactttccagttttcttatggaaccaaagaaacttcctacggcctgtccttcttcaagccacagcctggaaacttcgagagaaatttctccgtaaacttatataaagttactgggcagttcccgtggagctcacttcgggagacagacagaggagtgtctgcagagtacagttttcccctgtggaagaccagtcacactgtcaagtgggagggtgtgtggcgggagctgggctgcctctcgaggactgcgtcgttcgctgtgcggaaggaaagtggacactcactgaagtcgtctctctcgcatgccatggtcatcgactctcgaaattcatctatcttgccaagaagaggggccttgttcaaagtcaaccaggagctggcaggctacactggaggagatgtgagcttcatcaaggaagactttgagcttcagctgaataagccgctcgccttggactcggtattctccacgtctctctggggtggaatgctggtgcccatcggtgacaagccatccagcattgctgacaggttttacctgggaggccccacgagtgtccgaggatttagcatgcacagcattggaccccagagtgaaggagattacctgggcggcgaggcctactgggctgggggcctgcacctctacaccccactgcccttccggccaggccagggtggcttcggagagcttttcagaactcactttttcctcaatgcgggcaacctgtgcaacctcaactatggtgagggccccaaagcccatatccggaagctagctgagtgcatccgctggtcctatggagcaggcgtcgtcctccgacttggcaacatcgctcggctggagctgaactactgcattcctatgggcgtgcaggggggcgacaggatttgtgatggtgtccagtttggagctgggattcggttcctgtaacttgagtcccaggggcagcttggatgagaaacaggcatttcccaggctccaagcctatttggaggtgacacggttgagtgcttctgggccggaatccttatgggaaatcacgtcaataaattttaaacgctcaaaaaaaaaaaaaaaaaagaacacaaaccaacaacacacaaacaacacaaacacaacaacaaaacaaatcggcgg

SEQ ID NO: 7 - protein, 469 aa, Mus musculus
MGTVHARSLEPLPSSGTDFGALGEEAEFVEVEPEAKQEILENKDVVVQHVHFDGLGRTKDDIIICEIGEVFKAKNLIEVMRRSHEAREKLLRLGIFRQVDVLIDTCHGEDALPNGLDVTFEVTELRRLTGSYNTMVGNNEGSMVLGLKLPNLLGRAEKVTFQFSYGTKETSYGLSFFKPQPGNFERNFSVNLYKVTGQFPWSSLRETDRGVSAEYSFPLWKTSHTVKWEGVWRELGCLSRTASFAVRKESGHSLKSSLSHAMVIDSRNSSILPRRGALFKVNQELAGYTGGDVSFIKEDFELQLNKPLALDSVFSTSLWGGMLVPIGDKPSSIADRFYLGGPTSVRGFSMHSIGPQSEGDYLGGEAYWAGGLHLYTPLPFRPGQGGFGELFRTHFFLNAGNLCNLNYGEGPKAHIRKLAECIRWSYGAGVVLRLGNIARLELNYCIPMGVQGGDRICDGVQFGAGIRFL

SEQ ID NO: 8 - protein, 469 aa, Homo sapiens
MGTVHARSLEPLPSSGPDFGGLGEEAEFVEVEPEAKQEILENKDVVVQHVHFDGLGRTKDDIIICEIGDVFKAKNLIEVMRKSHEAREKLLRLGIFRQVDVLIDTCQGDDALPNGLDVTFEVTELRRLTGSYNTMVGNNEGSMVLGLKLPNLLGRAEKVTFQFSYGTKETSYGLSFFKPRPGNFERNFSVNLYKVTGQFPWSSLRETDRGMSAEYSFPIWKTSHTVKWEGVWRELGCLSRTASFAVRKESGHSLKSSLSHAMVIDSRNSSILPRRGALLKVNQELAGYTGGDVSFIKEDFELQLNKQLIFDSVFSASFWGGMLVPIGDKPSSIADRFYLGGPTSIRGFSMHSIGPQSEGDYLGGEAYLGRRWHLYTPLPFRPGQGGFGELFRTHFFLNAGNLCNLNYGEGPKAHIRKLAECIRWSYGAGIVLRLGNIARLELNYCVPMGVQTGDRICDGVQFGAGIRFL

SEQ ID NO: 9 - protein, 463 aa, Drosophila melanogaster
MPKSGRDGGASKDSKYDLSKISARVDRVNVSGLLRTHNDYVMRAADGLFKASNFQDLMLEAMSTKSYLHELGIFKDVSVHIDVSRGADASPQGYEVTFKGNEMSRMMGSAGTEIGQNEGSLRTELTIPNILGRGENISLQGSYSSTRANDLQLKFWKPFFHTRFKENRPEMSFSIFRQTDRFDISSFQTTNIGYLVDFSAHTMVGVDVSLKILQSKLNFLAIYFNFQLTHSLQYENAIRDVGLLNKSVPFAIRDHCGPKLASLLRYSVVYDNRDGNVFPTRGIYLKSVNEYCGLGGNVAYTSSTAHGELNVPLFAGLVAQFCARVGVVKETKNTTQLPISSLFYCGGPLTLRGFKFGGAGPVVESTPIGAQSFWCTGAHLWAPLPFAGVFKNLASHFRMHFFYNIGNNNSFSTENMRSAFGMGLAVKLAERARIELNYCVPVRHQDTDRILNGFQFGIGYEFV

SEQ ID NO: 10 - protein, 398 aa, Caenorhabditis elegans
MSEKTFHKAQTIRAKASGVPSIVEAVQFHGVRITKNDALVKEVSELYRSKNLDELVHNSHLAARHLQEVGLMDNAVALIDTSPSSNEGYVVNFLVREPKSFTAGVKAGVSTNGDADVSLNAGKQSVGGRGEAINTQYTYTVKGDHCFNISAIKPFLGWQKYSNVSATLYRSLAHMPWNQSDVDENAAVLAYNGQLWNQKLLHQVKLNAIWRTLRATRDAAFSVREQAGHTLKFSLENAVAVDTRDRPILASRGILARFAQEYAGVFGDASFVKNTLDLQAAAPLPLGFILAASFQAKHLKGLGDREVHILDRCYLGGQQDVRGFGLNTIGVKADNSCLGGGASLAGVVHLYRPLIPPNMLFAHAFLASGSVASVHSKNLVQQLQDTQRVSAGFGEFEI

SEQ ID NO: 11 - DNA, 447 bp, Mus musculus
ccatggagcacaaggtgatctgtgtcctcgctgtggtcctcatgctggccttcggcagtcttgcccaggcccaggcccaggcccaggcccaggaagaaacatgtatcatggccccccgggagaggataaattgtggcttccccggtgtcaccgcccagcagtgcacggagagaggttgctgttttgatgacagtgtccggggattcccgtggtgcttccaccccatggccatcgagaacactcaagaagaagaatgtcccttctaaggtccatcctgagagaactggctacatcaagacttggcaccctccacctgggcactggagccacctggcccacctgctacatacacacctattctgtggctggatctggctggtgacacagttcaacccctcagacttttagtcctcgaattcggcctgagaattaaaagagatgaatgtt

SEQ ID NO: 12 - protein, 87 aa, Mus musculus
MEHKVICVLAVVLMLAFGSLAQAQAQAQAQEETCIMAPRERINCGFPGVTAQQCTERGCCFDDSVRGFPWCFHPMAIENTQEEECPF

SEQ ID NO: 13 - protein, 81 aa, Rattus norvegicus
MEHKVTCVLAMVLMLALSSLAQNQEETCAVIPRERINCGFPGVTAQQCKEKGCCFDDSVRGFPWCFRPLVIENQQEEECPF

SEQ ID NO: 14 - protein, 84 aa, Homo sapiens
MATMENKVICALVLVSMLALGTLAEAQTETCTVAPRERQNCGFPGVTPSQCANKGCCFDDTVRGVPWCFYPNTIDVPPEEECEF

SEQ ID NO: 15 - protein, 78 aa, Xenopus laevis
MNYKVFCLVAIALIVGSIGSANGQAAFTEEQCSVERLARVNCGYSGITPQECTKQGCCFDSTIQDAPWCFYPRATPEC

SEQ ID NO: 16 - protein, 80 aa, Sus sp.
  MISC_FEATURE (64)..(64): X is any amino acid
MEARMFWLLVVLLALASSSSAGEYVGLSANQCAVPAKDRVDCGYPQVTPEQCNNRGCCFDSSIXGVPWCFKPLQETECTF

SEQ ID NO: 17 - DNA, 8680 bp, Homo sapiens
  misc_feature (933)..(933): n is a, t, c or g
gaacagggtccgggggaaccccctcaaatgaaaaggagccagggtaaacaagttacaaaagagaggagccagggtaaaaccagtagacgagaacaagtggaccgcagaggacacatccaggagccttcaggtacccatggccgcatgaagtgcagctttgataggaagctaaaatctcagaacacagaactgatgaaccttacaaatgagttttccccaaatggacttatgatccaagtgtaccagaaccagcaccttgggtgaacagtgagatttcttcaacagtgcctcaggtgggcatggagtaatggatttaaagcgattctgtcttactgaggccagtcagcactccaaggatgggagtcacaagttgtgattgggcaaagtttattttctatgtcagcttgtcagtcggctgcaccattttgcaagaatttttttggccttgataaaatgttctcagttaagtatggaagtttttctactgcttaatccaaaaagctattaaatcttactgaaataaaaataaatgcataaatacaatgcattattaataactgtggtcaccatgctgtacaatagatcgtaaaggatattcctccagggtcattgaaactttgtaccctttgaccacatttcctttcaacttagtcactgacttgctgatgttcggctttgacagaaatgcgtttccagaatgcctttccattcctccctcagagcagggagggcagttaggcttgcttctttcacttttaagttgattctttcctctgagggatggagtctgtcatccttacattcatcatccgctgggggttagcactccaaagtcaccctcacccaatttggcaaatccaccatttggcaaatccagcttctgtcagatgtgcccacatgtttgtttcttctccacaaatggaagaactacgngtgattactgctgctcactaaattaaacttaaanncaatctagtttattccagcaggttcctggtaaacangcactgcagctgaanaaaggcttatcctgtttatgttaacatgaaatatccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntaccacgcccagctaatcttttgtatttttagtagagacaggtttcatcatattggccaggctggtcccaaactcctgacctcaggtgatccacctgcttcagcctcccaaaatgtagggattacaggcatgagccaccactccctgccaaaaataatttttaagactaattttagcctacaaattatctgtgattttgttattttaggtaagttggagaggctaactaataagagatatagaaacaaccatcactaaatgacgtattcaaatgaacactgaagcccaacacatcgctttacaaacaaatcacactccttgtcccagacctagcgctccaaattcccaaaaatcaaataagtgttttttgttgttgttgtttgttttgctttgttttgggacagagtctcactctgtcgcccaggctggagtgcagtagtgtgatcttggctcactgcaacctccgtctcttgggttcaagcgattcacctgcctcagcctcctgagtagctgggattacaggcgcatgccatcacacctggccaatttttgtatttttagtagagacggggttttgccatgttggccaggctggtctcgaactcctgacctgcagtgatcagcctgtttcagcctccaaaagtgctaggattgcaggcatgagccaccgtgcccagcagcattttttttttaaaaagcttatctatcatttgaaccctcgaagtgatgacattttttggtaagtatttcagagaaaaaattgcttgtctacacttctaagaggatttgaggcagcaacattgcattacagttatttgttaatgctattagattgtaagctgttagaggcaggaatgccctctcttatgccttgtatcttccgttgcttaggccatattgctcatattattaatagctgttcaataaatgagttgatttaaattaaattttatcccaatgagttcatactgtttgcaatatatttctaaattgaaactgccaatctttaaaacaaaaaagagtccggtaccgagcttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctacaaaggactgtgcagccataaaaaagaagtttcactttttcactttaccattttccttaaaatttcccatggatggtattcttggtttttggggtggaagagagagagggagaagggaaacaggaaggaaatatttcaagggggagcaaagttgaattaagactggtggttgaaggctggcgatgaatgctccatctcttacttctctccttccattaatgtctaaggggaagcaacagaaaaggtcggttctgagtaggggaagcaaaggcagagcagtcccgctgctgtcatcagatttagcgagatttttgcccagctgtttgctgaaagaaaagcaggaaaacgaagaaccagagggctcaggcagggccactctgtcttgaatggtctcattcctcattctcaaagagaatctttcaggggttagctcttccttgagaatttctgcagctcagaaacctgccctttgaagcccagggtggccaccttttttttttttctttaattttttgagacggagtctagctctgttgcccagcaggctggagtgaagtggcgccatctcagctcattgcaacctctgcctcctggttgcaagcaattctcctgcctcagcctcccgagcagctgcaactacaggcatgcatcaccacgcctggctaatttttgtatttttagtagagacggggtttcaccatgttggacaggctggttttgaactcctgacctcaggtgatccacctgcctcggcctcccaaagtgctgggattacaggtgtgagccactgtgcctggccatttttttttttggaaagggcaatctgtgaccctcccactggctgccctgggagggttgtcagctttgttgtcaggatgttttatgttggacacttataggttgagttcaggccatcttgtggccttcctttagttttancacaggcagtttctgttcccaaaaagaaggggatagttttgggaacagctattgtcatccttgctttaaggtaaaccataaactaaattccttccaaagtagcttgcctatgcgcatgaatgataannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctataagctcggtacccctgtaatctcagttactcaggagattgaggcagaagaattgcttgaacccaggaggcagatgttgcagtgagccgagatcacgccactgtgctctaacctgggcgacagagcaagactccatcttgaaaaacaaaagaccaaaaagacaaaaacatgtttccatcttaggatagactgcccctcccattgctggccaattctgggatacagcaaagggctcaactgggagtgtgccctaggtatcccaactgcccaatccaggggccatatctcctctatctggtccatgcaccccaggaagcaatattcctctgccttaatcatcccagggccagctaccaggcaactagggacatgcttatagcttacagcttgctgaaattattcaaactagccaatcctagtttgtctttcctatggaaacttcaataaagactctagcataaaccatttacttgcttctatcttctgcttcctgaccacccaggtgtctttccttgtggccctgagtggcctgctgtagctcctgtctctaagactgtgagtataatgcactttgttttcctgagcctctcctctgtctcctcttgtggtggggacctggctgaacatttcataaaagaaggcaaaacagttgaccatgggcttgggacttacatacgcctggccaaagcttttgggatgagggctcagggaactatgtgccctgtgctacttctctgcttgtcatcttccccctcccacaaagcctattttctatctgtatcatgaggcataaatgatgtgtgtctatgactcctttgtaaaacagtttccaagttttgcttattaaccactcctgcatttgaggataaagtgacgtctgtggtgacagatcatgggattaataacaaatggttggaccactgctttcttcccacttaataaatattccatgtgtaattgaatcctgttgaattagttgggccacaaaaggcttcttcctcctttgctaggaatgcagccagagttgtgctttaaaagacacgtgtgcatgtgcttacactgaacaatccctggcaaaagaagtgtgaggagttagaacgccaaaaagcgaagagccagaaggtttcgtaaagcctttttttcatatggggtttttcccaaagggaaagcgtgggggccactcggtttcgggaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagagactatgcagacaccacaccttgagaagacctgtgaccttccgtagatgcacacagaggttccaggcaacctatctggcagggagccaggggaatcaatatttgacctcattcatcccttccttctcatctcctcctgggattccttcttcactggacaaacccagaggatgaagacatggaaacactttgtgggtccatacaggccagtttttctgagcagaaagacagatgcaaaaaggtggagaatggggaggggagatggagggaccagggacaaaaggaggctggcacagcagttgaactatctttagaacagaatttgtccctcttgaatttcttcatctgtctctgaatctcttttctacctttatgtacatgacctgttagcagacctttagaagttaagtactaagtttattctgctttttttattttttcaattttggggacagagttccaaaggcatctcacaaattcactccacgctcaagtcatttttccatcagcatatggcccttgggaagatcatatttgaaaagggatattttcaaaaagggtatttcaaatactttcaaaatgcatactcaaaaaagatcatatttcttgccatttggggaaataggtgtgtgtaagtaccttcaactcttcctaaataaagtgctttgaatctgcagcctgggtctgaggggcagtcttgttggtaattccctccccagcttacccagcagggccacaagtggtcttgcatcggaagcttggaagaaaacagcagtgatgccctgggttgaatgttcctactccactgaattgttacttgatttccccctaatttctttctagaattatattttaaggcctattattttattccatcaggaccatgagataaaatgttcaatcaccccangctaaagggaagaaacnnattcctggggcagcagtcttctctctacaccagcacattcagcaagcctttggcctctgagaggtcctagactcccctggaggttcagtaactcatcaaccttcaaatttcaaacccttagagtatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagaaactgagcctggtaccaaatgtgaattcaggccctacccagactcctgcctcacagtgcatttttccattagagtttgaaagcagtgctcttctcctgggaacctaggctgcctccaagagtctagtgaacactttcaggctctgccaagatgttcctactgtcaggatttcaaaatcgagttaaaactgagagaaaaaccaaggtgagtgtagagtgattccctttggtgcctgctttcaaacgagaagctaacaatgaacagttagaaaggaaagtaatgttttcatggtcaggggtgacaagccttgtctttaaacctcttagatgcccgttttaccctcatggtagagactgcaaggttgtccccgttctaaccaaactcctctgctttcttcataatagaatcccttgtatttcactagtgcttaatgtccaaaatagagaccacattactagtcttccctgaaagtaggatatctatgtgtaagttttagccaacactataaaagcaaaggtgcttcttgcaacttctaggaagtgtccttgtaagaatagaatatggctgggcgtggtggctcacgtctgtaatcccagcactttgggaggccgaggagggcagatcacttgagtccaggagttcaagaccagcctggacaagatggtgaaaccccgactttactaaaaaaaaaaaagattaaagaataaaacgaaataaatacaatgggctgactctgtacacgtgagctgcagcatggcagcccaccactccagcccactcaagatgcagaataaagctcataaaagtgggcagcatcggggtcggggatctgcacagcaggatggcaagggccatctagcactgaaaaccctaagcaagaaggtgagaaaagaactcagcagagtagaccagaggcattccgccagtcagctccaaaagcagaagaaggaggtggtcctggcggagaagagacagctgggtagcaaggatggctctcctcattagcactagctgtccagggattatctggcctcccactgaagaaacaattagatcccagaaagaagctaagtaaagcagtggagaagcatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctatgcgcaggaatgaattaaggacagcccagagattagaagcaagatggagtcagctatgtcagatttctcttactatcataattttgcaaaggtggtttcagtgggggtggtagtgaccagtatatctgtcagtggttttcatatactactgctcataagaatgcccttttggagcttggtaaaatacagacttctaggatcctgacaatcactttaaatgtaacaggtatacagtatacaggtatacagtaacagatgtctgatttttatagcaggcactctgagtaatttctgataaaggtggtcaaggagcagcattttgagaaatattggtaccctgtgcctaggcagtctgtgtgggtgagggtctcgacctctcagatcgtaaatttacagagatcctgtgttctgggatatgtgagtttagatagtctggtttaattaattaaaggagacacatattgctaatctttgtggctcaggaaaattttatcaaaggtacattttcctccattttggagttacagcggttttgttcttgcaggaagtttgagggtgtcttaatcagctcagctgccataacaaaaggccatgaaatgcagtggcttagatatagaacatgtatttctcacagctcttgaaagagtggaaaatctgagatcagggtgccaacatggttggatttatggaaaagggcactcttcctggcttgcagaatagacctccttctacagccagacattcttgctgtgttacccacatgcagagagggagtgagtcccaatctctaccacattcttataaagacattaatcccatcatgagaatttaatcctcatgatctcatctcaaccaaatggcctcccaaaggctccatgtgaaataccatcacactaggggttagggcttcaacatatgacttttagcggaacccaaacattcaatccataacaggagattctgcatcactaagaatggtactggctgattaacaagaggtttggtgtggatagaggtggatgtgagggcaggggcaggggagaatcagggaagggaagaaggagaaggggcgaggcagaagctgatctctagtgggcattgctgcatgcagtcatggtgctggccattgtagaggtgtcacttcatttaacatggtggaggtagtggggggtgtttcattttacctcagacatttgttttaatattctgtttgcacatattaaatatttaatctacaaaagggttggaggaaagaaatggcttttctgatgaccgagtaccaggactcctttttgcctcttcatcttcataccatgtgcactgcctgaaaggtgttcttccttcccatcctcctgcctccttcccattcccagctctgcatctccagttcggtctttgaatcctctgtactaataaaggcctagttcaagtgctaggtagatttctctgtcacccttttcaggagtcccagatcgtagccccttgcatggattcaatccttcattcatgtggataagtttcccagtttctgcaatcagcactgggattcaggttaccgacctacta

SEQ ID NO: 18 - DNA, 70 bp, Mus musculus
tgatcagcaagcagcattgttcggacaaatgtgcgtagaagttggacaagctaaaaacacttatggtacc

SEQ ID NO: 19 - DNA, 447 bp, Mus musculus; identical to SEQ ID NO: 11

(19) United States
(12) Patent Application Publication
(10) Pub. No.: US 2003/0208057 A1
(43) Pub. Date: Nov. 6, 2003

(54) Mammalian genes modulated during fasting and feeding

(76) Inventors: David A. Lewin, New Haven, CT (US); Timothy A. Stewart, San Francisco, CA (US)

Correspondence Address: SONNENSCHEIN NATH & ROSENTHAL, P.O. BOX 061080, WACKER DRIVE STATION, CHICAGO, IL, 60606-1080, US

(21) Appl. No.: 10/166,349

(22) Filed: May 7, 2002

Related U.S. Application Data
(60) Provisional application No. 60/306,969, filed on Jul. 20, 2001.

(51) Int. Cl. C12N 9/20; C07H 21/04; C12P 21/02; C12N 5/06

(52) U.S. Cl. 536/23.2; 435/198; 435/320.1; 435/325; 435/69.1

(57) ABSTRACT

Genes are disclosed that are differentially-regulated during feeding and fasting cycles. These genes, and their encoded polypeptides are useful to combat obesity.

### BACKGROUND

Obesity

Obesity is the most prevalent metabolic disorder in the United States, afflicting over a third of the population. While obesity can be simply defined as an excess of subcutaneous fat in proportion to lean body mass, relating to calorie intake and use, underlying metabolic disorders also contribute substantially. The assertion of metabolic dysfunction is confirmed by the observation that many obese people eat the same number of calories from similar sources as non-obese people. In obesity, adipocytes (fat cells) first increases in size, then number, as a person gains weight. A remedy—changing calorie intake and increasing calorie demand—can be daunting to those faced with metabolic troubles.

Metabolic issues that are to some degree inherited include a low resting metabolism, feeding behavior, changes in energy expenditures in response to overeating, and the basal rate of the breakdown of fat. In some instances, the mechanisms that signal the body that sufficient food has been eaten are defective (satiety mechanisms).

The cost of obesity is enormous to both the obese person as well as to society. Hundreds of thousands of deaths each year can be contributed to obesity or its associated complications, negatively impacting society. Treatment and care costs tens of billions of dollars. Personally, however, the physical well-being is adversely effected; obese persons are more likely to suffer from injuries, type II diabetes mellitus, hypertension, coronary heart disease, hypercholesterolemia, osteoarthritis, gallstones, cancers of the reproductive organs, sleep apnea (episodes of not breathing, correlating to higher incidence of stroke and heart attack), hypoventilation syndrome, osteoarthritis and other orthopedic disorders, infertility, lower extremity venous stasis disease, gastroesophageal reflux disease, and urinary stress incontinence (Pi-Sunjer and NHLBI Obesity Education Initiative Expert Panel on the Identification, Publication No. 98-4083, 1998; Report, 1997). Even medical procedures, such as surgeries, are less likely to succeed and are more often fraught with complications. Psychological costs are also high: the obese person may suffer from undeserved social stigmas, poor self-image, and psychological stress.

Biology of Obesity

Understanding obesity has been hampered by the absence of an animal model that immediately reflects the human situation. Human obesity does not generally follow a Mendelian inheritance pattern, wherein a single gene determines the obese phenotype (physical manifestation of a gene's expression) (Weigle and Kuijper, 1996), although there are several rodent models that do (Spiegelman and Flier, 1996; Weigle and Kuijper, 1996). Human obesity is a quantitative trait, with a few rare exceptions (Clement et al., 1998; Montague et al., 1997); that is, many genes contribute to the obese phenotype (Comuzzie and Allison, 1998). In fact, environmental and behavioral aspects also contribute (Hill and Peters, 1998). Thus a multi-front battle must be waged to conquer or reign-in this disorder (Perusse and Bouchard, 1999; Pi-Sunjer and NHLBI Obesity Education Initiative Expert Panel on the Identification, Publication No. 98-4083, 1998; Report, 1997).

Candidate genes that significantly influence obesity can be divided into groups based on phenotype (Table 1). These genes are candidates implicated directly in obesity in animal models, such as leptin encoded by the ob gene in mice, or are suspected to have a role in an obesity disorder.

TABLE 1
Selected candidate genes for human obesity/body composition (Comuzzie
and Allison, 1998)

| Phenotype | Gene | Notes |
|---|---|---|
| Obesity | agouti signaling | In mutant (a^{y}/a^{y}) mice, agouti is expressed |
| | polypeptide | ubiquitously (instead of only skin in wild-type |
| | | mice), and antagonizes melanocyte stimulating |
| | | hormone receptor ligation. |
| | carboxypeptidase | In agouti (a^{y}/a^{y}) mice, a mutation in this |
| | | enzyme prevents processing of |
| | | proopiomelanocortin. |
| | leptin | In mice, encoded by ob gene; mutant |
| | | homozygotes express the obese diabetic (db) |
| | | mouse phenotype due to aberrant leptin |
| | | translational termination. |
| | leptin receptor | fa/fa (fatty) rats (Phillips et al., 1996) |
| | tubby polypeptide | May effect processing of other obesity-related |
| | | polypeptides: neuropeptide Y and POMC |
| | | (Aron et al., 1997; Guan et al., 1998; |
| | | Spiegelman and Flier, 1996; Weigle and |
| | | Kuijper, 1996). |
| | proopiomelanocortin | Knock-out mice express a phenotype |
| | (POMC) | resembling that of agouti mutants (Yaswen et |
| | | al., 1999). |
| | tumor necrosis factor-α | Genetic linkage study of Pima Indians; up- |
| | | regulated in adipose tissue in obese people and |
| | | rodents (Norman et al., 1995). |
| Energy balance | uncoupling polypeptides | Uncoupling proteins disengage ATP synthesis |
| | (1, 2, 3) | from mitochondrial respiration, thereby |
| | | affecting metabolic rate (Schrauwen et al., |
| | | 1999). |
| Satiety | cholecystokinin A and its | (CCK) stimulates secretion of digestive enzyme |
| | receptor | and promotes cell growth (Cancela, 2001). |
| Feeding | melanocortin and its | Appetite suppressants, control feeding |
| behavior | receptors (3, 4) | behavior, among many other diverse functions |
| | | (Wikberg et al., 2000). |
| Appetite | neuropeptide Y | Promote feeding, although knockout mice |
| regulation | neuropeptide Y receptor | expressed a weaker phenotype than expected. |
| | | Double mutant mice, such as ob/ob npy-/npy- |
| | | have more striking phenotypes (Beck, 2001). |
| | ghrelin | Stimulates feeding and weight gain in mice |
| | | (Nakazato et al., 2001). |
| | other orexins | Stimulate feeding, e.g. (Sakurai et al., 1998a; |
| | | Sakurai et al., 1998b). |
| Adipocyte | peroxisome proliferator | Adipogenic transcription factor (Kersten, |
| differentiation | activated receptor-γ | 2001). |
| | β-3-adrenergic receptor | Expressed mostly in adipocytes. May be |
| | | coupled to lipolysis (Strosberg, 1997). |

Available Treatments

Non-pharmaceutical interventions include diet, exercise, and psychiatric and surgical treatment to optimize calorie intake/calorie output load or physically remove fat. Pharmaceuticals include mostly appetite suppressants and energy expenditure/nutrient-modifying agents. However, these treatments are often unsatisfactory, due to either unwanted complications, difficulties in maintaining weight loss after treatment, and/or unwanted side effects.

Although many candidate genes have been described, and others suggested by linkage analyses (Comuzzie and Allison, 1998), their usefulness to treat obesity in humans has often met with only limited success. For example, leptin (an appetite-suppressing hormone) administration as a treatment for obesity has entered clinical studies. In such a study examining the relationship between increased leptin dose and weight loss in both lean and obese adults (Heymsfield et al., 1999), only those obese subjects that received the highest dose (0.10-0.30 mg/kg/day) showed weight loss, although some obese subjects actually gained weight under this treatment regime. Furthermore, leptin administration was by daily subcutaneous injection, producing enough side effects that after the first 4 weeks of the 28 week study, almost a third of obese subjects declined to continue. Leptin's efficacy is at best moderate and besieged with complications. Leptin administration will most likely benefit those individuals that lack functional leptin (Farooqi et al., 1999), or suffer from other disorders, such as diabetes (Ebihara et al., 2001).

While there are many known candidate genes that may contribute to obesity (Table 1), other targets for various therapies are desirable. Optimal targets include those genes that are differentially-regulated during fasting and feeding because of their immediate relationship to food intake. These genes, along with their expression regulatory elements and encoded polypeptides represent a class of molecules that are desirable therapeutic targets and are also useful in predicting treatment success by expression profiling.

### SUMMARY

### DESCRIPTION OF THE DRAWINGS

FIG. 1 Hydrophobicity plot of glycerol kinase polypeptide (GLK; SEQ ID NO: 2)

FIG. 2 ClustalW alignment with GLK

FIG. 3 Hydrophobicity plot of peroxisomal membrane associated polypeptide (PMAP; SEQ ID NO: 7)

FIG. 4 ClustalW alignment with PMAP

FIG. 5 Hydrophobicity plot of Trefoil factor 1/pS2 secreted polypeptide (TFF1; SEQ ID NO: 12)

FIG. 6 ClustalW alignment with TFF1

### DETAILED DESCRIPTION

The present invention includes three genes that are remarkably differentially-regulated during fasting-feeding cycles, representing important weapons in the arsenal to treat and predict treatment success in obese subjects. These genes are useful in treating obesity, as markers for obesity diagnosis or propensity, and prognosis of the potential success of various treatment plans.

To identify those genes that are differentially regulated during fasting-feeding cycles, mice were put on various feeding regimes and at pre-determined time points mRNA was isolated from the stomach. Expression levels in fasting and feeding mice were then assessed and compared to identify those mRNA that were either up- or down regulated, using GeneCalling experiments (Shimkets et al., 1999) (see Examples), and the homology searches, such as BLAST (Altschul et al., 1997) were carried out to define the encoded polypeptide. In one set of experiments, three molecules were identified that were differentially expressed: (1) a glycerol kinase (GLK; Tables 2 and 3; down regulated during fasting, with a transient up regulation; post-fasting feeding, GLK is transiently upregulated and then down regulated), (2) a putative peroxisomal membrane associated polypeptide (PMAP; Tables 6 and 7; down regulated in response to feeding after fasting and induced with fasting), and (3) Trefoil Factor 1/pS2 secreted peptide (TFF1; Tables 10 and 11; down regulated in response to feeding after fasting), a known molecule previously unsuspected of playing any role in metabolism, especially obesity,. but well-characterized in oncogenic cells (Wright et al., 1997).

These differentially expressed genes, mRNAs and polypeptides can be manipulated in a variety of ways to treat obesity. Those genes that are up regulated during feeding, such as GLK, encode molecules that play roles in metabolic rate, satiety, and appetite suppression, and/or signal for the expression and/or activation of molecules that play such roles. For example, if a molecule up regulated during feeding signals satiety, then increased expression of this gene, administration of the polypeptide (or its active fragments) to obese subjects that habitually overeat can aid the subject in diminishing the quantity of food that they need to feel satisfied. On the other hand, genes down regulated during feeding, such as PMAP and TFF1, represent those molecules that signal feeding; up regulating their activity constitutively will also encourage obese subjects who eat too frequently to refrain. Likewise, differentially regulated genes during fasting may represent those molecules that signal or effect metabolic rate; those that accelerate metabolic rate could be up regulated in treatment to enhance the caloric utilization.

Together, the molecules GLK, PMAP and TFF1 are referred to collectively as DFF (differentially-regulated genes during fasting and feeding) molecules.

### I. Embodiments

The following embodiments are given as examples of various ways to practice the invention. Many different versions will be immediately apparent to one of skill in the various arts to which this invention pertains.

Obesity Treatment

DFFs can be exogenously regulated via a variety of means well-known in the art to treat or prevent obesity and other metabolic disorders, including: gene therapy techniques (including cell transformation of polynucleotides encoding active portions of a gene, anti-sense oligonucleotides), small molecule antagonists and agonists, polypeptide administration (for example, in replacement therapies), antibody administration to inhibit ligand-receptor interactions, etc.

Diagnostic and Prognostic Tools

Another application for differentially regulated genes is treatment prognosis and diagnosis. For example, if an obese subject constitutively expresses a gene that should be differentially regulated, such as a DFF, then treatments can be designed that target the expression and/or activity of that particular polypeptide. If an obese subject's expression profile (the totality of all, or preferably, a subset containing genes known to be differentially regulated during fasting and feeding, such as DFFs) is aberrant when compared to a lean individual, then a skilled artisan can determine which genes represent therapeutic targets, thus allowing many targets to be identified simultaneously. Finally, such expression profiling can diagnose the susceptibility of a subject to become obese.

DFF Molecules

The GLK and PMAP of the present invention includes the nucleic acids whose sequences comprise those provided in Tables 1 or 6 (GLK and PMAP, respectively) or fragments thereof. Mutant or variant GLKs or PMAPs, any of whose bases may be changed from the corresponding base shown in Tables 1 and 6 while still encoding a polypeptide that maintains the an activity or a physiological function of the GLK or PMAP fragment, or a fragment of such a nucleic acid, are also useful. Furthermore, nucleic acids, or fragments, whose sequences are complementary to those of Tables 1 and 6, are also advantageous. The invention additionally includes nucleic acids or nucleic acid fragments, or their complements, whose structures include chemical modifications. Such modifications include modified bases, and nucleic acids whose sugar phosphate backbones are modified or derivatized. These modifications are carried out at least in part to enhance the chemical stability of the modified nucleic acid, such that they may be used, for example, as anti-sense binding nucleic acids in therapeutic applications. In the mutant or variant nucleic acids, and their complements, up to 20% or more of the bases may be so changed.

The invention also includes polypeptides and nucleotides having 80-100%, including 81, 82, 83, 84, 85, 86, 87, 88, 89, 90, 91, 92, 93, 94, 95, 96, 97, 98 and 99%, sequence identity to the sequences presented in Tables 1 or 6, as well as nucleotides encoding any of these polypeptides, and compliments of any of these nucleotides.

The novel polypeptides of the invention include the polypeptide fragments whose sequences comprise those provided in Tables 2 or 7 and fragments thereof. The invention also includes GLK or PMAP mutant or variant polypeptides, any residues of which may be changed from the corresponding residue shown in Tables 2 and 7, while still encoding a polypeptide that maintains a native activity or physiological function, or a functional fragment thereof. In the mutant or variant GLK or PMAP, up to 20% or more of the residues may be so changed.

The invention further encompasses Abs and antibody fragments, such as F_{ab} or (F_{ab})′₂, that bind immunospecifically to any of the DFFs of the invention.

### II. Differentially Expressed Molecules During Fasting and Feeding

To distinguish between genes (and related nucleic acids) and the polypeptides that they encode, the abbreviations for genes are indicated by italicized (or underlined) text while abbreviations for the polypeptides are not italicized. Thus, GLK (glycerol kinase) or GLK refers to the nucleotide sequence that encodes GLK. Likewise, DFF refers collectively to nucleic acids related to GLK, PMAP (putative membrane associated polypeptide) and TFF1 (Trefoil Factor 1), and DFF refers to the polypeptides encoded by DFF or fragments thereof (Demerec et al., 1966).

Glycerol Kinase

In experiments examining gene expression during fasting and feeding, GLK mRNA was found to have a complex pattern of modulation: fasting induced down-regulation but was followed by a transient up-regulation; and, after post-fasting feeding, transient up-regulation (first 24 hours of feeding), and then down-regulation between 24 and 48 hours of post-fasting feeding.

Glycerol kinases catalyze the reaction of ATP and glycerol to yield sn-glycerol 3-phosphate and ADP; in adipose tissue, glycerol kinases are the first (and rate-limiting) step in triacylglycerol synthesis. Glycerol kinase-deficient individuals, such as those that result from the expression of an X-chromosome linked recessive allele, may experience disruptions in adrenal, muscle, and/or liver and brain function, and may also suffer from hypoglycaemia with hyperketonaemia and life-threatening metabolic events (Sjarif et al., 2000). GLK is a glycerol kinase. Because GLK's gene expression correlates with feeding and fasting, GLK is a non-redundant gene in triacylglycerol or other fat/adipose-related metabolism. The GLK genes, the GLK polypeptides, or molecules that interact with GLK genes and polypeptides are good drug targets for treating metabolic diseases, such as diabetes, obesity, cachexia, and anorexia in addition to its usefulness as a marker for monitoring metabolic phenomena. As a drug target, the action or expression of GLK during fasting can be up regulated, especially in subjects in whom GLK expression and/or activity is down regulated during feeding, permitting a subject to feel satisfied. Conversely, in individuals that are dangerously below weight, GLK expression and/or activity can be down regulated, encouraging feeding.

Table 2 shows the polynucleotide (DNA) sequence of GLK. The start and stop codons are indicated by boldface and underlining, respectively.

TABLE 2
GLK nucleotide sequence

(SEQ ID NO:1)

| | |
|---|---|
| tcaagaatat gctttgcctt attgcctgtg actttctgag attcaattat agtatctgtt | 60 |
| aaattctaat gttaaagaga actctttttt ccgctttgtg taagttaacc tatattgatt | 120 |
| accaatatca aataaaaagg tcctgtaatg agaataatca cctttaacct cctcggcaaa | 180 |
| acagcaaagc gtatgccata tcatagcgtg tcgcagcgcg aatttgagca aatctaccca | 240 |
| aaaccaggtt gggtagaaca cgacccaatg gaaatctggg ccacccaaag ctccacgctg | 300 |
| gtagaagtgc tggcgaaagc cgatatcagt tccgatcaaa ttgcagctat cggtattacg | 360 |
| aaccagcgtg aaaccactat tgtctgggaa aaagaaaccg gcaagcctat ctataacgcc | 420 |
| attgtctggc agtgccgtcg taccgcagaa atctgcgagc atttaaaacg tgacggttta | 480 |
| gaagattata tccgcagcaa taccggtctg gtgattgacc cgtacttttc tggcaccaaa | 540 |
| gtgaagtgga tcctcgacca tgtggaaggc tctcgcgagc gtgcacgtcg tggtgaattg | 600 |
| ctgtttggta cggttgatac gtggcttatc tggaaaatga ctcagggccg tgtccatgtg | 660 |
| accgattaca ccaacgcctc tcgtaccatg ttgttcaaca tccatatccc tggactggga | 720 |
| cgacaaatgc tggaagtgct ggatattccg cgcgagatgc tgccagaagt gcgtcgttct | 780 |
| tccgaagtat acggtcagac taacattggc ggcaaaggcg gcacgcgtat tccaatctcc | 840 |
| gggatcgccg gtgaccagca ggccgcgctg tttggtcagt tgtgcgtgaa agaagggatg | 900 |
| gcgaagaaca cctatggcac tggctgcttt atgctgatga acactggcga gaaagcggtg | 960 |
| aaatcagaaa acggcctgct gaccaccatc gcctgcggcc cgactggcga agtgaactat | 1020 |
| gcgttggaag gtgcggtgtt tatggcaggc gcatccattc agtggctgcg cgatgaaatg | 1080 |
| aagttgatta acgacgccta cgattccgaa tatttcgcca ccaaagtgca aaacaccaat | 1140 |
| ggtgtgtatg tggttccggc atttaccggg ctgatttacc gggctggtgc gccgtactgg | 1200 |
| gacccgtatg cgcgcggggc gattttcgtc tactcgtggg tgaacgctaa ccacattata | 1260 |
| cagcgcatcg ctgggttcca ggctagctat tctgtattga atgcggaact gtgcgtgccg | 1320 |
| caaacatcga tgcgggggcc gaagggcgtg cggtggtgaa tctcaaccgc ttcgacctcg | 1380 |
| ccatgctgaa accgtttatg ccagaaacca ctcaggccag cggtatcttc acgggtaaag | 1440 |
| cggacgttgc ctgggacacc acgaaagagg ggctgccgca gggcagtatc accnnnccgg | 1500 |
| ttttgtcgcc aatgatgccg cccaccagcg caccgagaaa cattccggcg gtcgtgattg | 1560 |
| ctgagaatgt ggctgtggtg gaattatctg tccagcccaa cgctttcagc tgcgcgagga | 1620 |
| tcaagccacc aacggcatta ctccagcaga caagcaagcc aaacgcgacg atggcaaaca | 1680 |
| ttgatgaatg ccagcggcaa tccggtaagc gatccagccg tgcaccacaa tgcggttaag | 1740 |
| ataatagctg gtacccatta cactggcgtt ttctcttcac gccagtcggt cgtgacttct | 1800 |
| gctaacaccg cagccggaga ttttccgttc aggcgcgtga cgcctgcttc tgattgcctg | 1860 |
| ctctccaggc agtggtcgcc ctgataaagc caggcgcgca gattggtcga tccccagtca | 1920 |
| attgcgatgt agcgagctgt catgtgattt cctttaacct tcgtgtcgag ctggcgatca | 1980 |
| tggtaa 1986 | |

Table 3 presents the GLK polypeptide amino acid sequence encoded by SEQ ID NO: 2.

TABLE 3
GLK polypeptide sequence

| | |
|---|---|
| Met Arg Ile Ile Thr Phe Asn Leu Leu Gly Lys Thr Ala Lys Arg Met | (SEQ ID NO:2) |
| 1 5 10 15 | |
| Pro Tyr His Ser Val Ser Gln Arg Glu Phe Glu Gln Ile Tyr Pro Lys | |
| 20 25 30 | |
| Pro Gly Trp Val Glu His Asp Pro Met Glu Ile Trp Ala Thr Gln Ser | |
| 35 40 45 | |
| Ser Thr Leu Val Glu Val Leu Ala Lys Ala Asp Ile Ser Ser Asp Gln | |
| 50 55 60 | |
| Ile Ala Ala Ile Gly Ile Thr Asn Gln Arg Glu Thr Thr Ile Val Trp | |
| 65 70 75 80 | |
| Glu Lys Glu Thr Gly Lys Pro Ile Tyr Asn Ala Ile Val Trp Gln Cys | |
| 85 90 95 | |
| Arg Arg Thr Ala Glu Ile Cys Glu His Leu Lys Arg Asp Gly Leu Glu | |
| 100 105 110 | |
| Asp Tyr Ile Arg Ser Asn Thr Gly Leu Val Ile Asp Pro Tyr Phe Ser | |
| 115 120 125 | |
| Gly Thr Lys Val Lys Trp Ile Leu Asp His Val Glu Gly Ser Arg Glu | |
| 130 135 140 | |
| Arg Ala Arg Arg Gly Glu Leu Leu Phe Gly Thr Val Asp Thr Trp Leu | |
| 145 150 155 160 | |
| Ile Trp Lys Met Thr Gln Gly Arg Val His Val Thr Asp Tyr Thr Asn | |
| 165 170 175 | |
| Ala Ser Arg Thr Met Leu Phe Asn Ile His Ile Pro Gly Leu Gly Arg | |
| 180 185 190 | |
| Gln Met Leu Glu Val Leu Asp Ile Pro Arg Glu Met Leu Pro Glu Val | |
| 195 200 205 | |
| Arg Arg Ser Ser Glu Val Tyr Gly Gln Thr Asn Ile Gly Gly Lys Gly | |
| 210 215 220 | |
| Gly Thr Arg Ile Pro Ile Ser Gly Ile Ala Gly Asp Gln Gln Ala Ala | |
| 225 230 235 240 | |
| Leu Phe Gly Gln Leu Cys Val Lys Glu Gly Met Ala Lys Asn Thr Tyr | |
| 245 250 255 | |
| Gly Thr Gly Cys Phe Met Leu Met Asn Thr Gly Glu Lys Ala Val Lys | |
| 260 265 270 | |
| Ser Glu Asn Gly Leu Leu Thr Thr Ile Ala Cys Gly Pro Thr Gly Glu | |
| 275 280 285 | |
| Val Asn Tyr Ala Leu Glu Gly Ala Val Phe Met Ala Gly Ala Ser Ile | |
| 290 295 300 | |
| Gln Trp Leu Arg Asp Glu Met Lys Leu Ile Asn Asp Ala Tyr Asp Ser | |
| 305 310 315 320 | |
| Glu Tyr Phe Ala Thr Lys Val Gln Asn Thr Asn Gly Val Tyr Val Val | |
| 325 330 335 | |
| Pro Ala Phe Thr Gly Leu Ile Tyr Arg Ala Gly Ala Pro Tyr Trp Asp | |
| 340 345 350 | |
| Pro Tyr Ala Arg Gly Ala Ile Phe Val Tyr Ser Trp Val Asn Ala Asn | |
| 355 360 365 | |
| His Ile Ile Gln Arg Ile Ala Gly Phe Gln Ala Ser Tyr Ser Val Leu | |
| 370 375 380 | |
| Asn Ala Glu Leu Cys Val Pro Gln Thr Ser Met Arg Gly Pro Lys Gly | |
| 385 390 395 400 | |
| Val Arg Trp | |

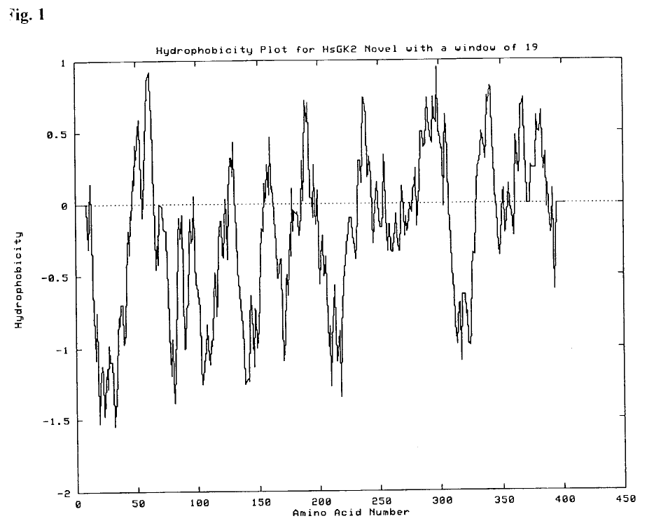

The predicted molecular weight of GLK, without post-translational modifications or alternative splicing, is 45,184.3 Da, with a predicted pI of 7.99. Table 4 presents other predicted physical characteristics of the GLK polypeptide (SEQ ID NO: 2). FIG. 1 presents a hydrophobicity plot of SEQ ID NO: 2 using a sliding window of 19 residues.

TABLE 4
Predicted physical properties of GLK¹

Wavelength

| | | | | |
|---|---|---|---|---|
| 276 nm | 278 nm | 279 nm | 280 nm | 282 nm |

Values assuming all Cys residues appear as half cystines

| | | | | | |
|---|---|---|---|---|---|
| Extinction | 84485 | 85781 | 85485 | 84710 | 82300 |
| Coefficient | | | | | |
| Optical | 1.870 | 1.898 | 1.892 | 1.875 | 1.821 |
| Density | | | | | |

Values assuming no Cys residues appear as half cystines

| | | | | | |
|---|---|---|---|---|---|
| Extinction | 84050 | 85400 | 85125 | 84350 | 82000 |
| Coefficient | | | | | |
| Optical | 1.860 | 1.890 | 1.884 | 1.867 | 1.815 |
| Density | | | | | |

¹Conditions at which these equations are valid are: pH 6.5, 6.0 M guanidium hydrochloride, 0.02 M phosphate buffer.

To ascertain cellular localization based on predicted stucture, the software PSORT (Nakai and Horton, 1999) was used, and the translocation sites were predicted to be mostly to peroxisomes, with some targeting to mitochondria. Such localization is consistent with GLK playing a role in metabolism since the peroxisome uses molecular oxygen to oxidize organic molecules and detoxify waste, and the mitochondria are charged with producing ATP. For example, persons lacking peroxisome organelles suffer from Zellweger syndrome, an infant lethal autosomal recessive disorder characterized by an accumulation of cis-4,7,10,13,16,19-docosahexaenoic acid (a 22 carbon polyunstaruated fatty acid) in membrane phopholipids. In this syndrome, neurons can not migrate, the child is afflicted by seizures, morphological defects, psychomotor retardation, hypotonia, myopathy, retinopathy, renal cortical cysts and hepatomegaly (Infante and Huszagh, 2001). Many other disorders are attributable to defective peroxisomes or peroxisomal molecules (Fujiki, 2000).

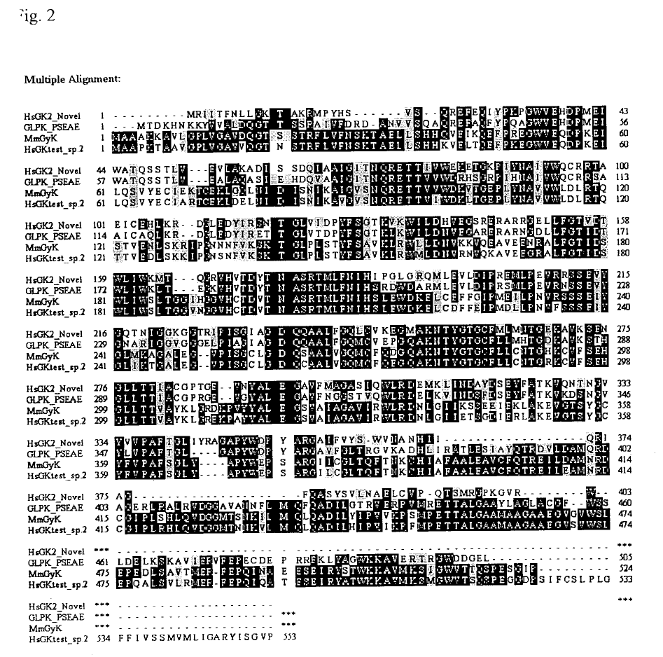

Homology to other molecules was found using BLAST (Altschul et al., 1990). CLUSTALW software for nearest neighbors (Thompson et al., 1994), the novel GLK (SEQ ID NO: 2, designated as “HsGK2_Novel” in FIG. 2), bacterial (Pseudomonas aeruginosa) glycerol kinase (GK) (SEQ ID NO: 3, “GLPK_PSEAE”), mouse GK (SEQ ID NO: 4; “MmGyK”, and a known human glycerol kinase (GK) (SEQ ID NO: 5; “HsGKtest_sp2”) were aligned (FIG. 2). Highly conserved regions (black) suggest those regions of the polypeptide that are most important for function; conservative amino acid substitutions (grey) also suggest important regions and properties (residue charge, size, etc.)

PMAP

PMAP is down regulated in response to post-fast feeding and is induced by fasting. The PMAP genes, the PMAP polypeptides, or molecules that interact with PMAP genes and polypeptides, are good drug targets for treating metabolic diseases, such as diabetes, obesity, cachexia, and anorexia in addition to its usefulness as a marker for monitoring metabolic phenomena. For example, obese (or individuals prone to obesity), PMAP expression and/or activity can be up regulated to discourage feeding or increase metabolism. Likewise, in individuals dangerously below weight, such as those suffering from, for example, anorexia, PMAP expression and/or activity can be down regulated to promote feeding or slow metabolism.

Table 5 shows the polynucleotide (DNA) sequence of PMAP. The start and stop codons are indicated by boldface and underlining, respectively; the polyadenylation signal is capitalized in boldface.

TABLE 5
Polynucleotide sequence of PMAP

(SEQ ID NO:6)

| | |
|---|---|
| gccgctgtac ggtccggaat tccgggacga cccacacgtc cgggcggacc ctaggtggta | 60 |
| caacatggcg gcgctcggca cgcagcttcc gtgacctgta gccgagcctc ttgtgtttgt | 120 |
| agctgagttt ggttgggcgc gggcaccgcc ttggggcagc agccggagag ggagccatgg | 180 |
| ggacagtgca cgcccggagt ctggagcccc tcccgtcgag tggaactgac tttggagcac | 240 |
| tgggggagga agccgagttt gtggaagttg agccggaagc gaaacaggaa atcctggaaa | 300 |
| acaaagatgt ggtggttcag catgttcatt ttgatggact tgggcggact aaggatgaca | 360 |
| tcatcatttg tgaaatcgga gaggtcttta aggctaaaaa cctcattgag gtaatgcggc | 420 |
| gatctcatga agcccgggaa aaactgcttc gcctaggaat ttttagacaa gtggatgttt | 480 |
| tgatcgatac atgtcatggt gaagatgccc tgcccaatgg gttagatgtc acctttgaag | 540 |
| tgacagagct gaggagactg acgggcagtt acaacaccat ggttggaaac aacgaaggca | 600 |
| gtatggtact cggcctcaaa ctccccaacc ttctgggacg agcagaaaaa gtcactttcc | 660 |
| agttttctta tggaaccaaa gaaacttcct acggcctgtc cttcttcaag ccacagcctg | 720 |
| gaaacttcga gagaaatttc tccgtaaact tatataaagt tactgggcag ttcccgtgga | 780 |
| gctcacttcg ggagacagac agaggagtgt ctgcagagta cagttttccc ctgtggaaga | 840 |
| ccagtcacac tgtcaagtgg gagggtgtgt ggcgggagct gggctgcctc tcgaggactg | 900 |
| cgtcgttcgc tgtgcggaag gaaagtggac actcactgaa gtcgtctctc tcgcatgcca | 960 |
| tggtcatcga ctctcgaaat tcatctatct tgccaagaag aggggccttg ttcaaagtca | 1020 |
| accaggagct ggcaggctac actggaggag atgtgagctt catcaaggaa gactttgagc | 1080 |
| ttcagctgaa taagccgctc gccttggact cggtattctc cacgtctctc tggggtggaa | 1140 |
| tgctggtgcc catcggtgac aagccatcca gcattgctga caggttttac ctgggaggcc | 1200 |
| ccacgagtgt ccgaggattt agcatgcaca gcattggacc ccagagtgaa ggagattacc | 1260 |
| tgggcggcga ggcctactgg gctgggggcc tgcacctcta caccccactg cccttccggc | 1320 |
| caggccaggg tggcttcgga gagcttttca gaactcactt tttcctcaat gcgggcaacc | 1380 |
| tgtgcaacct caactatggt gagggcccca aagcccatat ccggaagcta gctgagtgca | 1440 |
| tccgctggtc ctatggagca ggcgtcgtcc tccgacttgg caacatcgct cggctggagc | 1500 |
| tgaactactg cattcctatg ggcgtgcagg ggggcgacag gatttgtgat ggtgtccagt | 1560 |
| ttggagctgg gattcggttc ctgtaacttg agtcccaggg gcagcttgga tgagaaacag | 1620 |
| gcatttccca ggctccaagc ctatttggag gtgacacggt tgagtgcttc tgggccggaa | 1680 |
| tccttatggg aaatcacgtc AATAAAtttt aaacgctcaa aaaaaaaaaa aaaaaagaac | 1740 |
| acaaaccaac aacacacaaa caacacaaac acaacaacaa aacaaatcgg cgg 1793 | |

Table 6 show the PMAP polypeptide amino acid sequence encoded by SEQ ID NO: 6.

TABLE 6
PMAP polypeptide sequence

| Met Gly Thr Val His Ala Arg Ser Leu Glu Pro Leu Pro Ser Ser Gly | (SEQ ID NO:7) |
|---|---|
| 1 5 10 15 | |
| Thr Asp Phe Gly Ala Leu Gly Glu Glu Ala Glu Phe Val Glu Val Glu | |
| 20 25 30 | |
| Pro Glu Ala Lys Gln Glu Ile Leu Glu Asn Lys Asp Val Val Val Gln | |
| 35 40 45 | |
| His Val His Phe Asp Gly Leu Gly Arg Thr Lys Asp Asp Ile Ile Ile | |
| 50 55 60 | |
| Cys Glu Ile Gly Glu Val Phe Lys Ala Lys Asn Leu Ile Glu Val Met | |
| 65 70 75 80 | |
| Arg Arg Ser His Glu Ala Arg Glu Lys Leu Leu Arg Leu Gly Ile Phe | |
| 85 90 95 | |
| Arg Gln Val Asp Val Leu Ile Asp Thr Cys His Gly Glu Asp Ala Leu | |
| 100 105 110 | |
| Pro Asn Gly Leu Asp Val Thr Phe Glu Val Thr Glu Leu Arg Arg Leu | |
| 115 120 125 | |
| Thr Gly Ser Tyr Asn Thr Met Val Gly Asn Asn Glu Gly Ser Met Val | |
| 130 135 140 | |
| Leu Gly Leu Lys Leu Pro Asn Leu Leu Gly Arg Ala Glu Lys Val Thr | |
| 145 150 155 160 | |
| Phe Gln Phe Ser Tyr Gly Thr Lys Glu Thr Ser Tyr Gly Leu Ser Phe | |
| 165 170 175 | |
| Phe Lys Pro Gln Pro Gly Asn Phe Glu Arg Asn Ph Ser Val Asn Leu | |
| 180 185 190 | |
| Tyr Lys Val Thr Gly Gln Phe Pro Trp Ser Ser Leu Arg Glu Thr Asp | |
| 195 200 205 | |
| Arg Gly Val Ser Ala Glu Tyr Ser Phe Pro Leu Trp Lys Thr Ser His | |
| 210 215 220 | |
| Thr Val Lys Trp Glu Gly Val Trp Arg Glu Leu Gly Cys Leu Ser Arg | |
| 225 230 235 240 | |
| Thr Ala Ser Phe Ala Val Arg Lys Glu Ser Gly His Ser Leu Lys Ser | |
| 245 250 255 | |
| Ser Leu Ser His Ala Met Val Ile Asp Ser Arg Asn Ser Ser Ile Leu | |
| 260 265 270 | |
| Pro Arg Arg Gly Ala Leu Phe Lys Val Asn Gln Glu Leu Ala Gly Tyr | |
| 275 280 285 | |
| Thr Gly Gly Asp Val Ser Phe Ile Lys Glu Asp Phe Glu Leu Gln Leu | |
| 290 295 300 | |
| Asn Lys Pro Leu Ala Leu Asp Ser Val Phe Ser Thr Ser Leu Trp Gly | |
| 305 310 315 320 | |
| Gly Met Leu Val Pro Ile Gly Asp Lys Pro Ser Ser Ile Ala Asp Arg | |
| 325 330 335 | |
| Phe Tyr Leu Gly Gly Pro Thr Ser Val Arg Gly Phe Ser Met His Ser | |
| 340 345 350 | |
| Ile Gly Pro Gln Ser Glu Gly Asp Tyr Leu Gly Gly Glu Ala Tyr Trp | |
| 350 360 365 | |
| Ala Gly Gly Leu His Leu Tyr Thr Pro Leu Pro Phe Arg Pro Gly Gln | |
| 370 375 380 | |
| Gly Gly Phe Gly Glu Leu Phe Arg Thr His Phe Phe Leu Asn Ala Gly | |
| 385 390 395 400 | |
| Asn Leu Cys Asn Leu Asn Tyr Gly Glu Gly Pro Lys Ala His Ile Arg | |
| 405 410 415 | |
| Lys Leu Ala Glu Cys Ile Arg Trp Ser Tyr Gly Ala Gly Val Val Leu | |
| 420 425 430 | |
| Arg Leu Gly Asn Ile Ala Arg Leu Glu Leu Asn Tyr Cys Ile Pro Met | |
| 435 440 445 | |
| Gly Val Gln Gly Gly Asp Arg Ile Cys Asp Gly Val Gln Phe Gly Ala | |
| 450 455 460 | |
| Gly Ile Arg Phe Leu | |
| 465 | |

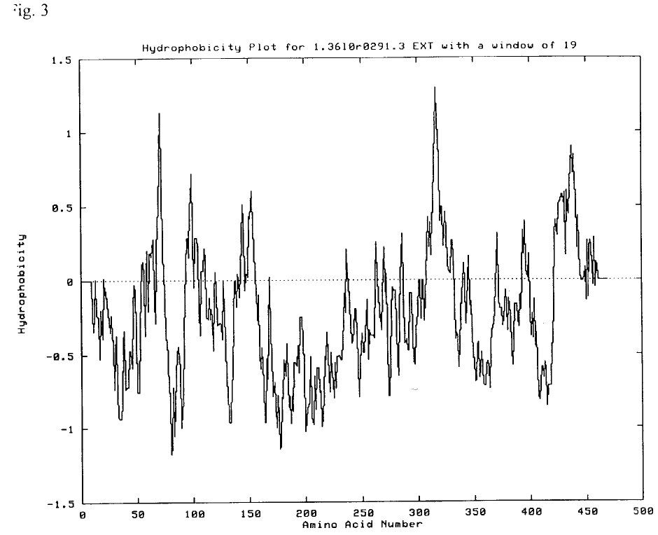

PMAP has a predicted (unprocessed) molecular weight of 51,863 Da and a pI of 6.78. Table 7 shows other predicted physical properties of PMAP (SEQ ID NO: 7). FIG. 3 presents a hydrophobicity plot of PMAP using a sliding window of 19 residues.

TABLE 7
Predicted physical properties of PMAP¹

Wavelength

| | | | | |
|---|---|---|---|---|
| 276 nm | 278 nm | 279 nm | 280 nm | 282 nm |

Values assuming all Cys residues appear as half cystines

| | | | | | |
|---|---|---|---|---|---|
| Extinction | 57085 | 57781 | 57465 | 56830 | 55100 |
| Coefficient | | | | | |
| Optical | 1.101 | 1.114 | 1.108 | 1.096 | 1.062 |
| Density | | | | | |

Values assuming no Cys residues appear as half cystines

| | | | | | |
|---|---|---|---|---|---|
| Extinction | 56650 | 57400 | 57105 | 56470 | 54800 |
| Coefficient | | | | | |
| Optical | 1.092 | 1.107 | 1.101 | 1.089 | 1.057 |
| Density | | | | | |

¹Conditions at which these equations are valid are: pH 6.5, 6.0 M guanidium hydrochloride, 0.02 M phosphate buffer.

Localization of PPMP based on predicted structure, PSORT indicated translocation to the peroxisome. Like GLK, localization to this cellular compartment is consistent for a role in metabolism for PMAP.

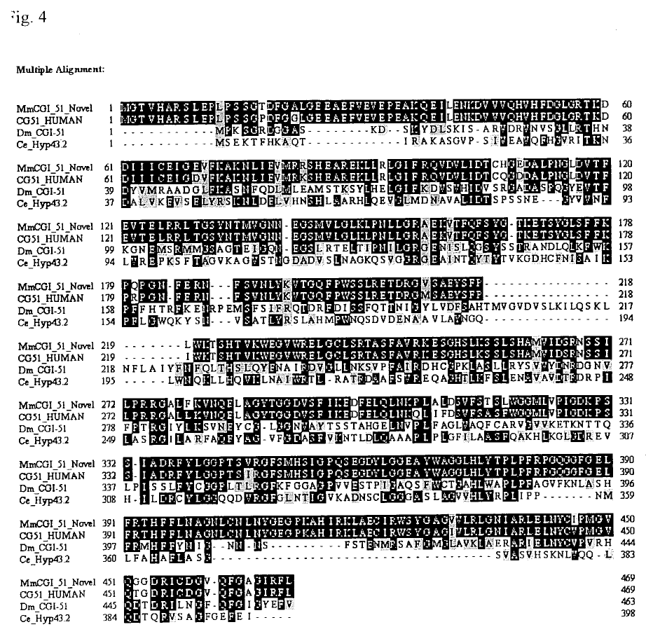

PMAP is a novel polypeptide with limited homology to other polypeptides. FIG. 4 shows a CLUSTALW of nearest neighbors of PMAP with closely-related polypeptides. In FIG. 4, MmCGI_{—}51_Novel is PMAP (SEQ ID NO: 7), CG51_HUMAN is a predicted polypeptide of unknown function with homology to bacterial surface antigen (SEQ ID NO: 8), Dm_CGI-51 (SEQ ID NO: 9) is the D. melanogaster CGI-51 homolog, and Ce_Hyp43.2 (SEQ ID NO: 10) is the C. elegans homolog. Highly conserved regions are shaded in black, and conservative amino acid substitutions are shaded in grey.

TFF1 (also known as pS2)

TFF1 is induced in fasting mice and down regulated upon post-fasting feeding. TFF1, also known as pS2, is secreted by stomach corpus mucous neck cells, superficial cells of the body, and antral mucosa. However, TFF1 has been most studied in the digestive tract (Wong et al., 1999).

TFFs, of which three are currently known in H. sapiens, have a trefoil or P domain that share a six cysteine residue motif, distinct from motifs found in other polypeptides. TFF1 localizes principally to ductal luminal cells of Brunner's glands of the small intestine, goblet cells near the surface of large intestine crypts, and all regions of the stomach in mucous cells from the stomach neck upwards (Wong et al., 1999). A knock-out mouse for TFF1 points to a role in gastric cell differentiation, and TFF1 may act as a tumor-suppressor (Lefebvre et al., 1996). Further studies have also pointed to functions in gastrointestinal injury mucosal repair (Playford et al., 1996). TFF1 is also expressed in a wide variety of human carcinomas (Wong et al., 1999). TFF1 may be capable of dimerization, forming heterodimers with other trefoil factors (TFF2 or TFF3), allowing them to interact with goblet cell mucous secretions, cross-linking the mucous and thus conferring a protective effect to the cells from gastric juices. In addition, trefoil factors are powerful mitogens, correlating with a role as a tumor suppressor or differentiation (Wong et al., 1999). A role in metabolism or obesity has never been suggested; that TFF1 is differentially regulated in response to fasting regimes is unexpected.

Because of its differential regulation in fasting (up regulated) vs. feeding (down regulated) mice, TFF1 polypeptides and/or TFF1-interacting polypeptides are useful as drugs or drug targets for treating metabolic diseases, including diabetes, obesity, cachexia and anorexia. TFF1 can also serve as a marker for monitoring metabolic phenomena. For example, in obese individuals (or individuals prone to obesity), TFF1 expression and/or activity can be up regulated to discourage feeding or increase metabolism. Likewise, in individuals dangerously below weight, such as those suffering from cachexia or anorexia, TFF1 expression and/or activity can be down regulated to promote feeding or slow metabolism.

Modulating TFF1 activity in subjects suffering from cachexia is especially important, given the association of TFF1 expression with carcinomas. Cachexia is a wasting phenomenon observed in almost half of all cancer patients, as well as individuals afflicted with other diseases, such as AIDS. In cancers, especially gastric and pancreatic, cachexia results when tumor-induced distant metabolic changes are disproportionate to tumor burden. Cachexia-induced weight loss can lead to respiratory distress: metabolic changes lead to loss of adipose tissue and skeletal muscle mass and weaken the diaphragm.

Table 8 shows the polynucleotide sequence of mouse TFF1. Start and stop codons are indicated by boldface and underlining, respectively.

TABLE 8
TFF1 nucleotide sequence

(SEQ ID NO:11)

| | |
|---|---|
| ccatggagca caaggtgatc tgtgtcctcg ctgtggtcct catgctggcc ttcggcagtc | 60 |
| ttgcccaggc ccaggcccag gcccaggccc aggaagaaac atgtatcatg gccccccggg | 120 |
| agaggataaa ttgtggcttc cccggtgtca ccgcccagca gtgcacggag agaggttgct | 180 |
| gttttgatga cagtgtccgg ggattcccgt ggtgcttcca ccccatggcc atcgagaaca | 240 |
| ctcaagaaga agaatgtccc ttctaaggtc catectgaga gaactggcta catcaagact | 300 |
| tggcaccctc cacctgggca ctggagccac ctggcccacc tgctacatac acacctattc | 360 |
| tgtggctgga tctggctggt gacacagttc aacccctcag acttttagtc ctcgaattcg | 420 |
| gcctgagaat taaaagagat gaatgtt 447 | |

Table 9 presents the TFF1 polypeptide amino acid sequence encoded by SEQ ID NO: 11. The Cys residues that form disulfide bonds to form the characteristic 3-looped structure are indicated in boldface, and the trefoil domain is indicated by upper-case residues.

TABLE 9
TFF1 polypeptide sequence

| Met Glu His Lys Val Ile Cys Val Leu Ala Val Val Leu Met Leu Ala | (SEQ ID NO:12) |
|---|---|
| 1 5 10 15 | |
| Phe Gly Ser Leu Ala Gln Ala Gln Ala Gln Ala Gln Ala Gln Glu Glu | |
| 20 25 30 | |
| Thr CYS ILE MET ALA PRO ARG GLU ARG ILE ASN CYS GLY PHE PRO GLY | |
| 35 40 45 | |
| VAL THR ALA GLN GLN CYS THR GLU ARG GLY CYS CYS PHE ASP ASP SER | |
| 50 55 60 | |
| VAL ARG GLY PHE PRO TRP CYS PHE HIS PRO MET ALA ILE GLU ASN THR | |
| 65 70 75 80 | |
| GLN GLU GLU GLU CYS Pro Phe | |
| 85 | |

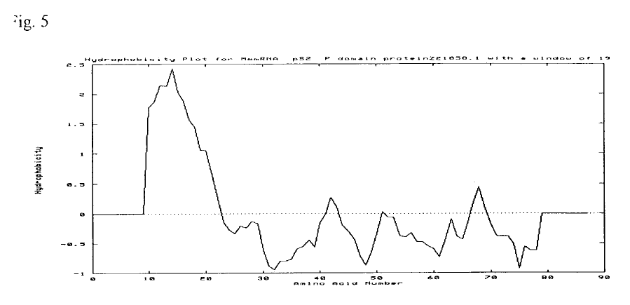

Disregarding possible post-translational processing or alternative splicing, mouse TFF1 has a predicted molecular weight of 9670.0 Da and a pI of 4.45. Consistent with the observation that TFF1 is secreted, PSORT analyses demonstrate an endoplasmic recitulum membrane and lumen, and the lysosome. A hydropathy plot is indicated in FIG. 5. Table 10 presents other predicted physical characteristics of the mouse TFF1 polypeptide.

TABLE 10
Predicted physical properties of TFF1¹

Wavelength

| | | | | |
|---|---|---|---|---|
| 276 nm | 278 nm | 279 nm | 280 nm | 282 nm |

Values assuming all Cys residues appear as half cystines

| | | | | | |
|---|---|---|---|---|---|
| Extinction | 5980 | 6108 | 6140 | 6170 | 6000 |
| Coefficient | | | | | |
| Optical | 0.618 | 0.632 | 0.635 | 0.638 | 0.620 |
| Density | | | | | |

Values assuming no Cys residues appear as half cystines

| | | | | | |
|---|---|---|---|---|---|
| Extinction | 5400 | 5600 | 5660 | 5690 | 5600 |
| Coefficient | | | | | |
| Optical | 0.558 | 0.579 | 0.585 | 0.588 | 0.579 |
| Density | | | | | |

¹Conditions at which these equations are valid are pH 6.5, 6.0 M guanidium hydrochloride, 0.02 M phosphate buffer.

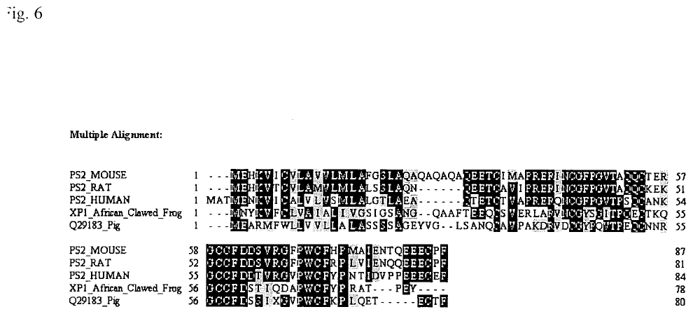

FIG. 6 shows a CLUSTALW of nearest neighbors of mouse TFF1 with TFF1 from other organisms. In FIG. 6, TFF1 from mouse (PS2_MOUSE; SEQ ID NO: 12), rat (PS2_RAT; SEQ ID NO: 13), human (PS2_HUMAN; SEQ ID NO: 14), African clawed frog (XP1_African_Clawed_Frog; SEQ ID NO: 15) and pig (Q29183_Pig; SEQ ID NO: 16) are aligned. Highly conserved regions are shaded in black, and conservative amino acid substitutions are shaded in grey.

### III. Practicing the Invention

Definitions

Unless defined otherwise, all technical and scientific terms have the same meaning as is commonly understood by one of skill in the art to which this invention belongs. The definitions below are presented for clarity.

“Isolated,” when referred to a molecule, refers to a molecule that has been identified and separated and/or recovered from a component of its natural environment. Contaminant components of its natural environment are materials that interfere with diagnostic or therapeutic use.

Nucleic Acid-Related Definitions

Probes

Probes are nucleic acid sequences of variable length, preferably between at least about 10 nucleotides (nt), 100 nt, or many (e.g., 6,000 nt) depending on the specific use. Probes are used to detect identical, similar, or complementary nucleic acid sequences. Longer length probes can be obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies. Probes are substantially purified oligonucleotides that will hybridize under stringent conditions to at least optimally12, 25, 50, 100, 150, 200, 250, 300, 350 or 400 consecutive sense strand nucleotide sequence of SEQ ID NOS: 1, 6 or 11; or an anti-sense strand nucleotide sequence of SEQ ID NOS: 1, 6 or 11; or of naturally occurring mutants of SEQ ID NOS: 1, 6 or 11.

The full- or partial length native sequence DFF may be used to “pull out” similar (homologous) sequences (Ausubel et al., 1987; Sambrook, 1989), such as: (1) full-length or fragments of DFF cDNA from a cDNA library from any species (e.g. human, murine, feline, canine, bacterial, viral, retroviral, or yeast), (2) from cells or tissues, (3) variants within a species, and (4) homologs, orthologues and variants from other species. To find related sequences that may encode related genes, the probe may be designed to encode unique sequences or degenerate sequences. Sequences may also be DFF genomic sequences including promoters, enhancer elements and introns.

For example, GLK coding region in another species may be isolated using such probes. A probe of about 40 bases is designed, based on mouse GLK (mGLK; SEQ ID NO: 1), and made. To detect hybridizations, probes are labeled using, for example, radionuclides such as ³²p or ³⁵S, or enzymatic labels such as alkaline phosphatase coupled to the probe via avidin-biotin systems. Labeled probes are used to detect nucleic acids having a complementary sequence to that of mGLK in libraries of cDNA, genomic DNA or mRNA of a desired species.

Probes can be used as a part of a diagnostic test kit for identifying cells or tissues which mis-express a DFF, such as by measuring a level of a DFF in a sample of cells from a subject e.g., detecting DFF mRNA levels or determining whether a genomic DFF has been mutated or deleted. Probes are also useful in arrays that allow for the simultaneous examination of multiple sequences.

Control Sequences

Control sequences are DNA sequences that enable the expression of an operably-linked coding sequence in a particular host organism. Prokaryotic control sequences include promoters, operator sequences, and ribosome binding sites. Eukaryotic cells utilize promoters, polyadenylation signals, and enhancers.

Operably-Linked

Nucleic acid is operably-linked when placed into a functional relationship with another nucleic acid sequence. For example, a promoter or enhancer is operably-linked to a coding sequence if it affects the transcription of the sequence, or a ribosome-binding site is operably-linked to a coding sequence if positioned to facilitate translation. Generally, “operably-linked” means that the DNA sequences being linked are contiguous, and, in the case of a secretory leader, contiguous and in reading phase. However, enhancers do not have to be contiguous. Linking can be accomplished by conventional recombinant DNA methods.

Isolated Nucleic Acids

An isolated nucleic acid molecule is purified from the setting in which it is naturally found and is separated from at least one contaminant nucleic acid molecule. Isolated DFF molecules are distinguished from the specific DFF molecule in cells. However, an isolated DFF molecule includes DFF molecules contained in cells that ordinarily express DFF where, for example, the nucleic acid molecule is in a chromosomal location different from that of natural cells.

Oligonucleotides

An oligonucleotide comprises a series of linked nucleotide residues, which oligonucleotide has a sufficient number of nucleotide bases to be useful, such as in PCR reactions or as probes. A short oligonucleotide sequence may be based on, or designed from, a genomic or cDNA DFF sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. An oligonucleotide comprising a nucleic acid molecule less than 100 nt in length would further comprise at least 6 contiguous nucleotides of SEQ ID NOS: 1, 6 or 11, or complements thereof. Oligonucleotides may be chemically synthesized.

Complementary Nucleic Acid Sequences; Binding

In another embodiment, an isolated nucleic acid molecule of the invention comprises a nucleic acid molecule that is a complement of the nucleotide sequence shown in SEQ ID NOS: 1, 6 or 11, or a portion of these sequences (e.g., fragments that can be used as a probes, primers or fragments encoding a biologically-active portion of a DFF). A nucleic acid molecule that is complementary to the nucleotide sequence shown in SEQ ID NOS: 1, 6 or 11, is one that is sufficiently complementary to the nucleotide sequence shown in SEQ ID NOS: 1, 6 or 11, that it can hydrogen bond with little or no mismatches to the nucleotide sequence shown in SEQ ID NOS: 1, 6 or 11, thereby forming a stable duplex.

“Complementary” refers to Watson-Crick or Hoogsteen base pairing between nucleotides of a nucleic acid molecule. “Binding” means the physical or chemical interaction between two polypeptides or compounds, or associated polypeptides, or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Nucleic acid fragments are at least 6 contiguous nucleic acids or at least 4 contiguous amino acids, a sufficient length to allow for specific hybridization in the case of nucleic acids or for specific recognition of an epitope in the case of amino acids, respectively, and are at most some portion less than a full-length sequence. Fragments may be derived from any contiguous portion of a nucleic acid or amino acid sequence of choice.

Derivatives and Analogs

Derivatives are nucleic acid sequences or amino acid sequences formed from native compounds either directly, by modification or partial substitution. Analogs are nucleic acid sequences or amino acid sequences that have a structure similar to, but not identical, the native compound but differ from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid. Derivatives or analogs of the nucleic acids or polypeptides of the invention include, but are not limited to, molecules comprising regions that are substantially homologous to DFF nucleic acids or polypeptides by at least about 70%, 80%, or 95% identity (with a preferred identity of 80-95%) over a nucleic acid or amino acid sequence of identical size or when compared to an aligned sequence in which the alignment is done by a well-known algorithm in the art, or whose encoding nucleic acid is capable of hybridizing to the complement of a sequence encoding the aforementioned polypeptides under stringent, moderately stringent, or low stringent conditions (Ausubel et al., 1987).

Homology

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of DFF. Isoforms can be expressed in different tissues of the same organism as a result of, for example, alternative splicing. Alternatively, different genes can encode isoforms. Homologous DFF nucleotide sequences of species other than mice, including other vertebrates, such as human, frog, rat, rabbit, dog, cat, cow, horse, and other organisms. Homologous nucleotide sequences also include naturally occurring allelic variations and mutations of SEQ ID NOS: 1, 6 or 11. A homologous nucleotide sequence does not, however, include the exact nucleotide sequence encoding mouse DFFs. Homologous nucleic acid sequences may encode conservative amino acid substitutions (see below) in SEQ ID NOS: 2, 7 or 12, as well as a polypeptide possessing DFF biological activity.

Open Reading Frames

The open reading frame (ORF) of a DFF gene encodes DFF. An ORF is a nucleotide sequence that has a start codon (ATG) and terminates with one of the three “stop” codons (TAA, TAG, or TGA). In this invention, however, an ORF may be any part of a coding sequence that may or may not comprise a start codon and a stop codon. To achieve a unique sequence, preferable DFF ORFs encode at least 50 amino acids.

Polypeptide-Related Definitions

Polypeptide, Polypeptides and Peptides

The terms polypeptide, peptide and polypeptide are well known in the art. A polypeptide has an amino acid sequence that is longer than a peptide. A peptide contains 2 to about 50 amino acid residues. The term polypeptide includes polypeptides and peptides. Examples of polypeptides include antibodies, enzymes, lectins and receptors; lipopolypeptides and lipopolypeptides; and glycopolypeptides and glycopolypeptides. Examples of polypeptides include neuropeptides, functional domains (e.g. PDZ domains) of polypeptides, peptides having 3-20 residues obtained from phage display libraries, etc.

Mature

A DFF can encode a mature DFF. A “mature” form of a polypeptide or polypeptide disclosed in the present invention is the product of a naturally occurring polypeptide or precursor form or propolypeptide. The naturally occurring polypeptide, precursor or propolypeptide includes the full-length gene product, encoded by the corresponding genomic sequence or open reading frame. The product “mature” form arises as a result of one or more processing steps as they may take place within the cell, or host cell, in which the gene product arises. Examples of such processing steps include the cleavage of the N-terminal methionine residue encoded by the initiation codon of an open reading frame, or the signal peptide cleavage or leader sequence. Thus a mature form arising from a precursor polypeptide or polypeptide that has residues 1 to n, where residue 1 is the N-terminal methionine, would have residues 2 through n after removal of the N-terminal methionine. Alternatively, a mature form arising from a precursor polypeptide or polypeptide having residues 1 to n in which an N-terminal signal sequence from residue 1 to residue m is cleaved, would have the residues from residue m+1 to residue n remaining. A “mature” form of a polypeptide or polypeptide may arise from other post-translational modifications, such as glycosylation, myristoylation or phosphorylation. In general, a mature polypeptide or polypeptide may result from the operation of only one of these processes, or a combination of any of them.

Purified Polypeptide

When the molecule is a purified polypeptide, the polypeptide will be purified (1) to obtain at least 15 residues of N-terminal or internal amino acid sequence using a sequenator, or (2) to homogeneity by SDS-PAGE under non-reducing or reducing conditions using Coomassie blue or silver stain. Isolated polypeptides include those expressed heterologously in genetically-engineered cells or expressed in vitro, since at least one component of the DFF natural environment is absent. Ordinarily, isolated polypeptides are prepared by at least one purification step.

Active Polypeptide

An active DFF or DFF fragment retains a biological and/or an immunological activity of native or naturally-occurring DFF. Immunological activity refers to the ability to induce the production of an antibody against an antigenic epitope possessed by a native DFF; biological activity refers to a function caused by a native DFF that excludes immunological activity. A GLK biological function includes kinase activity (phosphorylating target molecules); for PMAP, a role in peroxisome function (such as oxidation) or peroxisome integrity, and for TFF1, a mitogenic or cross-linking activity.

Epitope Tags

An epitope tagged polypeptide refers to a chimeric polypeptide fused to a “tag polypeptide”. Such tags provide epitopes against which Abs can be made or are available, but do not interfere with polypeptide activity. To reduce anti-tag antibody reactivity with endogenous epitopes, the tag polypeptide is preferably unique. Suitable tag polypeptides generally have at least six amino acid residues, usually between about 8 and 50 amino acid residues, preferably between 8 and 20 amino acid residues. Examples of epitope tag sequences include HA from Influenza A virus and FLAG.

DFF Nucleic Acid Variants and Hybridization

Variant Polynucleotides, Genes and Recombinant Genes

The invention further encompasses nucleic acid molecules that differ from the nucleotide sequences shown in SEQ ID NOS: 1, 6 or 11 due to degeneracy of the genetic code and thus encode same GLK, PMAP or TFF1 as that encoded by the nucleotide sequences shown in SEQ ID NOS: 1, 6 or 11. An isolated nucleic acid molecule of the invention has a nucleotide sequence encoding a polypeptide having an amino acid sequence shown in SEQ ID NOS: 2, 7 or 12.

In addition to the DFF sequences shown in SEQ ID NOS: 1, 6 or 11, DNA sequence polymorphisms that change the DFF amino acid sequences may exist within a population. For example, allelic variations among individuals exhibit genetic polymorphisms in DFFs. The terms “gene” and “recombinant gene” refer to nucleic acid molecules comprising an open reading frame (ORF) encoding a DFF. Such natural allelic variations can typically result in 1-5% variance in DFF. Any and all such nucleotide variations and resulting amino acid polymorphisms in the DFF, which are the result of natural allelic variation and leave intact DFF functional activity are within the scope of the invention.

Moreover, DFF from other species that have a nucleotide sequence that differs from the sequence of SEQ ID NOS: 1, 6 or 11 are contemplated. Nucleic acid molecules corresponding to natural allelic variants and homologs of DFF cDNAs can be isolated based on their homology to SEQ ID NOS: 1, 6 or 11 using cDNA-derived probes to hybridize to homologous DFF sequences under stringent conditions.

“DFF variant polynucleotide” or “DFF variant nucleic acid sequence” means a nucleic acid molecule which encodes an active DFF that (1) has at least about 80% nucleic acid sequence identity with a nucleotide acid sequence encoding a full-length native DFF, (2) a full-length native DFF lacking the signal peptide, (3) an extracellular domain of a DFF, with or without the signal peptide, or (4) any other fragment of a full-length DFF. Ordinarily, a DFF variant polynucleotide will have at least about 80% nucleic acid sequence identity, more preferably at least about 81%, 82%, 83%, 84%, 85%, 86%, 87%, 88%, 89%, 90%, 91%, 92%, 93%, 94%, 95%, 96%, 97%, 98% nucleic acid sequence identity and yet more preferably at least about 99% nucleic acid sequence identity with the nucleic acid sequence encoding a full-length native DFF. A DFF variant polynucleotide may encode full-length native DFF lacking the signal peptide, an extracellular domain of a DFF, with or without the signal sequence, or any other fragment of a full-length DFF. Variants do not encompass the native nucleotide sequence.

Ordinarily, DFF variants are at least about 30 nucleotides, often at least about 60, 90, 120, 150, 180, 210, 240, 270, 300, 450, 600 nucleotides in length, more often at least about 900 nucleotides in length, or more.

“Percent (%) nucleic acid sequence identity” with respect to DFF-encoding nucleic acid sequences is defined as the percentage of nucleotides in the DFF sequence of interest that are identical with the nucleotides in a candidate sequence, after aligning the sequences and introducing gaps, if necessary, to achieve the maximum percent sequence identity. Alignment can be achieved in various ways well-known in the art; for instance, using publicly available software such as BLAST, BLAST-2, ALIGN or Megalign (DNASTAR) software. Those skilled in the art can determine appropriate parameters for measuring alignment, including any necessary algorithms to achieve maximal alignment over the full length of the sequences being compared.

When nucleotide sequences are aligned, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) can be calculated as follows:

% nucleic acid sequence identity=W/Z·100

where

W is the number of nucleotides scored as identical matches by the sequence alignment program's or algorithm's alignment of C and D and

Z is the total number of nucleotides in D.

When the length of nucleic acid sequence C is not equal to the length of nucleic acid sequence D, the % nucleic acid sequence identity of C to D will not equal the % nucleic acid sequence identity of D to C.

Stringency

Homologs (i.e., nucleic acids encoding DFF derived from species other than human) or other related sequences (e.g., paralogs) can be obtained by low, moderate or high stringency hybridization with all or a portion of the particular human sequence as a probe using methods well known in the art for nucleic acid hybridization and cloning.

The specificity of single stranded DNA to hybridize complementary fragments is determined by the “stringency” of the reaction conditions. Hybridization stringency increases as the propensity to form DNA duplexes decreases. In nucleic acid hybridization reactions, the stringency can be chosen to either favor specific hybridizations (high stringency), which can be used to identify, for example, full-length clones from a library. Less-specific hybridizations (low stringency) can be used to identify related, but not exact, DNA molecules (homologous, but not identical) or segments.

DNA duplexes are stabilized by: (1) the number of complementary base pairs, (2) the type of base pairs, (3) salt concentration (ionic strength) of the reaction mixture, (4) the temperature of the reaction, and (5) the presence of certain organic solvents, such as formamide which decreases DNA duplex stability. In general, the longer the probe, the higher the temperature required for proper annealing. A common approach to achieve different stringencies is to vary the temperature: higher relative temperatures result in more stringent reaction conditions. Ausubel et al. (1987) provide guidance and an excellent explanation of stringency of hybridization reactions. To hybridize under “stringent conditions” describes hybridization protocols in which nucleotide sequences at least 60% homologous to each other remain hybridized.

(a) High Stringency

“Stringent hybridization conditions” conditions enable a probe, primer or oligonucleotide to hybridize only to its target sequence. Stringent conditions are sequence-dependent and will differ. Stringent conditions comprise: (1) low ionic strength and high temperature washes (e.g. 15 mM sodium chloride, 1.5 mM sodium citrate, 0.1% sodium dodecyl sulfate at 50° C.); (2) a denaturing agent during hybridization (e.g. 50% (v/v) formamide, 0.1% bovine serum albumin, 0.1% Ficoll, 0.1% polyvinylpyrrolidone, 50 mM sodium phosphate buffer (pH 6.5; 750 mM sodium chloride, 75 mM sodium citrate at 42° C.); or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other.

(b) Moderate Stringency

“Moderately stringent conditions” use washing solutions and hybridization conditions that are less stringent (Sambrook, 1989), such that a polynucleotide will hybridize to the entire, fragments, derivatives or analogs of SEQ ID NOS: 1, 6 or 11. One example comprises hybridization in 6×SSC, 5×Denhardt's solution, 0.5% SDS and 100 mg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions are described (Ausubel et al., 1987; Kriegler, 1990).

(c) Low Stringency

“Low stringent conditions” use washing solutions and hybridization conditions that are less stringent than those for moderate stringency (Sambrook, 1989), such that a polynucleotide will hybridize to the entire, fragments, derivatives or analogs of SEQ ID NOS: 1, 6 or 11. An example of low stringency hybridization conditions is hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency, such as those for cross-species hybridizations are described (Ausubel et al., 1987; Kriegler, 1990; Shilo and Weinberg, 1981).

Conservative Mutations

In addition to naturally-occurring allelic variants of DFF, changes can be introduced by mutation into SEQ ID NOS: 1, 6 or 11 that incur alterations in the amino acid sequences of DFF but do not alter DFF function. For example, nucleotide substitutions leading to amino acid substitutions at “non-essential” amino acid residues can be made in SEQ ID NOS: 2, 7 or 12. A “non-essential” amino acid residue is a residue that can be altered from the wild-type sequences of the DFF without altering their biological activity, whereas an “essential” amino acid residue is required for biological activity. For example, amino acid residues that are conserved among the DFF of the invention are predicted to be particularly non-amenable to alteration (Tables 5, 10 and 13).

Useful conservative substitutions are shown in Table A, “Preferred substitutions.” Conservative substitutions whereby an amino acid of one class is replaced with another amino acid of the same type fall within the scope of the subject invention so long as the substitution does not materially alter the biological activity of the compound. If such substitutions result in a change in biological activity, then more substantial changes, indicated in Table B as exemplary are introduced and the products screened for DFF biological activity.

TABLE A
Preferred substitutions

| Original | | Preferred |
|---|---|---|
| residue | Exemplary substitutions | substitutions |
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro, Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Leu |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, Norleucine | Leu |

Non-conservative substitutions that effect (1) the structure of the polypeptide backbone, such as a β-sheet or α-helical conformation, (2) the charge or (3) hydrophobicity, or (4) the bulk of the side chain of the target site can modify DFF polypeptide function or immunological identity. Residues are divided into groups based on common side-chain properties as denoted in Table B. Non-conservative substitutions entail exchanging a member of one of these classes for another class. Substitutions may be introduced into conservative substitution sites or more preferably into non-conserved sites.

TABLE B
Amino acid classes

| Class | Amino acids |
|---|---|
| hydrophobic | Norleucine, Met, Ala, Val, Leu, Ile |
| neutral hydrophilic | Cys, Ser, Thr |
| acidic | Asp, Glu |
| basic | Asn, Gln, His, Lys, Arg |
| disrupt chain conformation | Gly, Pro |
| aromatic | Trp, Tyr, Phe |

The variant polypeptides can be made using methods known in the art such as oligonucleotide-mediated (site-directed) mutagenesis, alanine scanning, and PCR mutagenesis. Site-directed mutagenesis (Carter, 1986; Zoller and Smith, 1987), cassette mutagenesis, restriction selection mutagenesis (Wells et al., 1985) or other known techniques can be performed on the cloned DNA to produce DFF variants (Ausubel et al., 1987; Sambrook, 1989).

Anti-Sense Nucleic Acids

Using antisense and sense DFF oligonucleotides can prevent DFF polypeptide expression. These oligonucleotides bind to target nucleic acid sequences, forming duplexes that block transcription or translation of the target sequence by enhancing degradation of the duplexes, terminating prematurely transcription or translation, or by other means.

Antisense or sense oligonucleotides are singe-stranded nucleic acids, either RNA or DNA, which can bind target DFF mRNA (sense) or DFF DNA (antisense) sequences. Anti-sense nucleic acids can be designed according to Watson and Crick or Hoogsteen base pairing rules. The anti-sense nucleic acid molecule can be complementary to the entire coding region of DFF mRNA, but more preferably, to only a portion of the coding or noncoding region of DFF mRNA. For example, the anti-sense oligonucleotide can be complementary to the region surrounding the translation start site of DFF mRNA. Antisense or sense oligonucleotides may comprise a fragment of the DFF coding region of at least about 14 nucleotides, preferably from about 14 to 30 nucleotides. In general, antisense RNA or DNA molecules can comprise at least 5, 10, 15, 20, 25, 30, 35, 40, 45, 50, 55, 60, 65, 70, 75, 80, 85, 90, 95, 100 bases in length or more. Methods to derive antisense or sense oligonucleotides are well described (Stein and Cohen, 1988; van der Krol et al., 1988a).

Examples of modified nucleotides that can be used to generate the anti-sense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the anti-sense nucleic acid can be produced using an expression vector into which a nucleic acid has been sub-cloned in an anti-sense orientation such that the transcribed RNA will be complementary to a target nucleic acid of interest.

To introduce antisense or sense oligonucleotides into target cells (cells containing a target nucleic acid sequence), any gene transfer method may be used. Examples of gene transfer methods include (1) biological, such as gene transfer vectors like Epstein-Barr virus or conjugating the exogenous DNA to a ligand-binding molecule, (2) physical, such as electroporation and injection, and (3) chemical, such as CaPO₄ precipitation and oligonucleotide-lipid complexes.

An antisense or sense oligonucleotide is inserted into a suitable gene transfer retroviral vector. A cell containing the target nucleic acid sequence is contacted with the recombinant retroviral vector, either in vivo or ex vivo. Examples of suitable retroviral vectors include those derived from the murine retrovirus M-MuLV, N2 (a retrovirus derived from M-MuLV), or the double copy vectors designated DCT5A, DCT5B and DCT5C (WO 90/13641, 1990). To achieve sufficient nucleic acid molecule transcription, vector constructs in which the transcription of the anti-sense nucleic acid molecule is controlled by a strong pol II or pol III promoter are preferred. Also preferred are tissue- and cell-specific promoters, when known.

To specify target cells in a mixed population of cells, cell surface receptors that are specific to the target cells can be exploited. Antisense and sense oligonucleotides are conjugated to a ligand-binding molecule, as described (WO 91/04753, 1991). Examples of suitable ligand-binding molecules include cell surface receptors, growth factors, cytokines, or other ligands that bind to target cell surface molecules. Preferably, conjugation of the ligand-binding molecule does not substantially interfere with the ability of the receptors or molecule to bind the ligand-binding molecule conjugate, or block entry of the sense or antisense oligonucleotide or its conjugated version into the cell.

Liposomes efficiently transfer sense or an antisense oligonucleotide to cells (WO 90/10448, 1990). The sense or antisense oligonucleotide-lipid complex is preferably dissociated within the cell by an endogenous lipase.

The anti-sense nucleic acid molecule of the invention may be an α-anomeric nucleic acid molecule. An α-anomeric nucleic acid molecule forms specific double-stranded hybrids with complementary RNA in which, contrary to the usual α-units, the strands run parallel to each other (Gautier et al., 1987). The anti-sense nucleic acid molecule can also comprise a 2′-o-methylribonucleotide (Inoue et al., 1987a) or a chimeric RNA-DNA analog (Inoue et al., 1987b).

An anti-sense nucleic acid may be a catalytic RNA molecule with ribonuclease activity, a ribozyme. For example, hammerhead ribozymes (Haseloff and Gerlach, 1988) can be used to catalytically cleave DFF mRNA transcripts and thus inhibit translation. A ribozyme specific for a DFF-encoding nucleic acid can be designed based on the nucleotide sequence of a DFF cDNA (i.e., SEQ ID NOS: 1, 6 or 11). For example, a derivative of a Tetrahymena L-19 IVS RNA can be constructed in which the nucleotide sequence of the active site is complementary to the nucleotide sequence to be cleaved in a DFF-encoding mRNA (Cech et al., U.S. Pat. No. 5,116,742, 1992; Cech et al., U.S. Pat. No. 4,987,071, 1991). DFF mRNA can also be used to select a catalytic RNA having a specific ribonuclease activity from a pool of RNA molecules (Bartel and Szostak, 1993).

Alternatively, DFF expression can be inhibited by targeting nucleotide sequences complementary to the regulatory region of a DFF (e.g., DFF promoter and/or enhancers) to form triple helical structures that prevent transcription of the DFF in target cells (Helene, 1991; Helene et al., 1992; Maher, 1992).

Modifications of antisense and sense oligonucleotides can augment their effectiveness. Modified sugar-phosphodiester bonds or other sugar linkages (WO 91/06629, 1991) increase in vivo stability by conferring resistance to endogenous nucleases without disrupting binding specificity to target sequences. Other modifications can increase the affinities of the oligonucleotides for their targets, such as covalently linked organic moieties (WO 90/10448, 1990) or poly-(L)-lysine. Other attachments modify binding specificities of the oligonucleotides for their targets, including metal complexes or intercalating (e.g. ellipticine) and alkylating agents.

For example, the deoxyribose phosphate backbone can be modified to generate peptide nucleic acids (Hyrup and Nielsen, 1996). “Peptide nucleic acids” (PNAs) refer to nucleic acid mimics in that the deoxyribose phosphate backbone is replaced by a pseudopeptide backbone, and only the four natural nucleobases are retained. The neutral backbone of PNAs allows for specific hybridization to DNA and RNA under conditions of low ionic strength. PNA oligomers can be synthesized using solid phase peptide synthesis protocols (Hyrup and Nielsen, 1996; Perry-O'Keefe et al., 1996).

PNAs of DFF can be used in therapeutic and diagnostic applications. For example, PNAs can be used as anti-sense or antigene agents for sequence-specific modulation of gene expression by inducing transcription or translation arrest or inhibiting replication. DFF PNAs may also be used in the analysis of single base pair mutations (e.g., PNA directed PCR clamping; as artificial restriction enzymes when used in combination with other enzymes, e.g., S₁ nucleases (Hyrup and Nielsen, 1996); or as probes or primers for DNA sequence and hybridization (Hyrup and Nielsen, 1996; Perry-O'Keefe et al., 1996).

DFF PNAs can be modified to enhance their stability or cellular uptake. Lipophilic or other helper groups may be attached to PNAs, PNA-DNA dimmers formed, or the use of liposomes or other drug delivery techniques. For example, PNA-DNA chimeras can be generated that combine the advantages of PNA and DNA. Such chimeras allow DNA recognition enzymes (e.g., RNase H and DNA polymerases) to interact with the DNA portion while the PNA portion provides high binding affinity and specificity. PNA-DNA chimeras can be linked using linkers of appropriate lengths selected in terms of base stacking, number of bonds between the nucleobases, and orientation (Hyrup and Nielsen, 1996). The synthesis of PNA-DNA chimeras are described (Finn et al., 1996; Hyrup and Nielsen, 1996). For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Finn et al., 1996; Hyrup and Nielsen, 1996). PNA monomers are then coupled in a stepwise manner to produce a chimeric molecule with a 5′ PNA segment and a 3′ DNA segment (Finn et al., 1996). Alternatively, chimeric molecules can be synthesized with a 5′ DNA segment and a 3′ PNA segment (Petersen et al., 1976).

The oligonucleotide may include other appended groups such as peptides (e.g., for targeting host cell receptors in vivo), or agents facilitating transport across the cell membrane (Lemaitre et al., 1987; Letsinger et al., 1989) or the blood-brain barrier (Pardridge and Schimmel, WO89/10134, 1989). In addition, oligonucleotides can be modified with hybridization-triggered cleavage agents (van der Krol et al., 1988b) or intercalating agents (Zon, 1988). The oligonucleotide may be conjugated to another molecule, e.g., a peptide, a hybridization triggered cross-linking agent, a transport agent, a hybridization-triggered cleavage agent, and the like.

DFF Polypeptides

The invention pertains to isolated DFFs, and biologically-active portions derivatives, fragments, analogs or homologs thereof. Also provided are polypeptide fragments suitable for use as immunogens to raise anti-DFF Abs. DFFs may be isolated from cells and tissues, produced by recombinant DNA techniques or chemically synthesized.

Polypeptides

A DFF polypeptide includes an amino acid sequence of DFF whose sequences are provided in SEQ ID NOS: 2, 7 or 12. The invention also includes a mutant or variant polypeptide any of whose residues may be changed from the corresponding residues shown in SEQ ID NOS: 2, 7 or 12, while still encoding an active DFF, or a functional fragment.

DFF Polypeptide Variants

In general, a DFF variant that preserves DFF-like function and includes any variant in which residues at a particular position in the sequence have been substituted by other amino acids, and further includes the possibility of inserting an additional residue or residues between two residues of the parent polypeptide as well as the possibility of deleting one or more residues from the parent sequence. Preferably, the substitution is a conservative substitution (Table A).

“DFF polypeptide variant” means an active DFF having at least: (1) about 80% amino acid sequence identity with a full-length native DFF sequence, (2) a DFF sequence lacking a signal peptide, (3) an extracellular domain of a DFF, with or without a signal peptide, or (4) any other fragment of a full-length DFF sequence. For example, DFF variants include those wherein one or more amino acid residues are added or deleted at the N- or C-terminus of the full-length native amino acid sequence. A DFF polypeptide variant will have at least about 80% amino acid sequence identity, preferably at least about 81% amino acid sequence identity, more preferably at least about 82%, 83%, 84%, 85%, 86%, 87%, 88%, 89%, 90%, 91%, 92%, 93%, 94%, 95%, 96%, 97%, 98% amino acid sequence identity and most preferably at least about 99% amino acid sequence identity with a full-length native sequence DFF sequence. Ordinarily, DFF variant polypeptides are at least about 10 amino acids in length, often at least about 20 amino acids in length, more often at least about 30, 40, 50, 60, 70, 80, 90, 100, 150, 200, or 300 amino acids in length, or more.

“Percent (%) amino acid sequence identity” is defined as the percentage of amino acid residues that are identical with amino acid residues in a DFF sequence in a candidate sequence when the two sequences are aligned. To determine % amino acid identity, sequences are aligned and if necessary, gaps are introduced to achieve the maximum % sequence identity; conservative substitutions are not considered as part of the sequence identity. Amino acid sequence alignment procedures to determine percent identity are well known to those of skill in the art. Publicly available computer software such as BLAST, BLAST2, ALIGN2 or Megalign (DNASTAR) can be used to align polypeptide sequences. Those skilled in the art will determine appropriate parameters for measuring alignment, including any algorithms needed to achieve maximal alignment over the full length of the sequences being compared.

When amino acid sequences are aligned, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as:

% amino acid sequence identity=X/Y·100

where

X is the number of amino acid residues scored as identical matches by the sequence alignment program's or algorithm's alignment of A and B and

Y is the total number of amino acid residues in B.

If the length of amino acid sequence A is not equal to the length of amino acid sequence B, the % amino acid sequence identity of A to B will not equal the % amino acid sequence identity of B to A.

Isolated/Purified Polypeptides

An “isolated” or “purified” polypeptide, polypeptide or biologically active fragment is separated and/or recovered from a component of its natural environment. Contaminant components include materials that would typically interfere with diagnostic or therapeutic uses for the polypeptide, and may include enzymes, hormones, and other polypeptideaceous or non-polypeptideaceous materials. Preferably, the polypeptide is purified to a sufficient degree to obtain at least 15 residues of N-terminal or internal amino acid sequence. To be substantially isolated, preparations having less than 30% by dry weight of contaminants, more preferably less than 20%, 10% and most preferably less than 5% contaminants. An isolated, recombinantly-produced DFF or biologically active portion is preferably substantially free of culture medium, i.e., culture medium represents less than 20%, more preferably less than about 10%, and most preferably less than about 5% of the volume of the DFF preparation. Examples of contaminants include cell debris, culture media, and substances used and produced during in vitro synthesis of DFF.

Biologically Active

Biologically active portions of DFF include peptides comprising amino acid sequences sufficiently homologous to, or derived from, the amino acid sequences of a DFF (SEQ ID NOS: 2, 7 or 12) that include fewer amino acids than the full-length DFF, and exhibit at least one activity of a DFF. Biologically active portions comprise a domain or motif with at least one activity of native DFF. For example, activities include kinase activity (GLK), peroxisome activity or integrity (PMAP), or mitogen activity (TTF1). A biologically active portion of a DFF can be a polypeptide that is 10, 25, 50, 100 or more amino acid residues in length. Other biologically active portions, in which other regions of the polypeptide are deleted, can be prepared by recombinant techniques and evaluated for one or more of the functional activities of a native DFF.

Biologically active portions of a DFF may have an amino acid sequence shown in SEQ ID NOS: 2, 7 or 12, or substantially homologous to SEQ ID NOS: 2, 7 or 12, and retains the functional activity of the polypeptide of SEQ ID NOS: 2, 7 or 12, yet differs in amino acid sequence due to natural allelic variation or mutagenesis. Other biologically active DFF may comprise an amino acid sequence at least 45% homologous to the amino acid sequence of SEQ ID NOS: 2, 7 or 12, and retains the functional activity of native DFF. Homology can be determined as described in DFF polypeptide variants, above.

Chimeric and Fusion Polypeptides

Fusion polypeptides are useful in expression studies, cell-localization, bioassays, and DFF purification. A DFF “chimeric polypeptide” or “fusion polypeptide” comprises DFF fused to a non-DFF polypeptide. A non-DFF polypeptide is not substantially homologous to DFF (SEQ ID NOS: 2, 7 or 12). A DFF fusion polypeptide may include any portion to an entire DFF, including any number of biologically active portions. In some host cells, heterologous signal sequence fusions may ameliorate DFF expression and/or secretion. Exemplary fusions are presented in Table C.

Other fusion partners can adapt DFF therapeutically. Fusions with members of the immunoglobulin (Ig) family are useful to inhibit DFF ligand or substrate interactions, consequently suppressing DFF-mediated signal transduction in vivo. DFF-Ig fusion polypeptides can also be used as immunogens to produce anti-DFF Abs in a subject, to purify DFF ligands, and to screen for molecules that inhibit interactions of DFF with other molecules.

Fusion polypeptides can be easily created using recombinant methods. A nucleic acid encoding DFF can be fused in-frame with a non-DFF encoding nucleic acid, to the DFF NH₂— or COO—-terminus, or internally. Fusion genes may also be synthesized by conventional techniques, including automated DNA synthesizers. PCR amplification using anchor primers that give rise to complementary overhangs between two consecutive gene fragments that can subsequently be annealed and reamplified to generate a chimeric gene sequence (Ausubel et al., 1987). Many vectors are commercially available that facilitate sub-cloning DFF in-frame to a fusion moiety.

TABLE C
Useful non-DFF fusion polypeptides and usefulness thereof

| Polypeptide | in vitro | in vivo | Notes | Reference |
|---|---|---|---|---|
| Human growth | Radioimmunoassay | none | Expensive, | (Selden et al., |
| hormone (hGH) | | | insensitive, | 1986) |
| | | | narrow linear | |
| | | | range. | |
| β-glucuronidase | Colorimetric, | colorimetric | sensitive, | (Gallagher, |
| (GUS) | fluorescent, or | (histo-chemical | broad linear | 1992) |
| | chemiluminescent | staining with X- | range, non- | |
| | | gluc) | isotopic. | |
| Green | Fluorescent | fluorescent | can be used in | (Chalfie et al., |
| fluorescent | | | live cells; | 1994) |
| polypeptide | | | resists photobleaching | |
| (GFP) and | | | | |
| related | | | | |
| molecules (RFP, | | | | |
| BFP, YFP, etc.) | | | | |
| Luciferase | bioluminescent | Bioluminescent | polypeptide is | (de Wet et al., |
| (firefly) | | | unstable, | 1987) |
| | | | difficult to | |
| | | | reproduce, | |
| | | | signal is brief | |
| Chloramphenicol | Chromatography, | none | Expensive | (Gorman et al., |
| acetyltransferase | differential | | radioactive | 1982) |
| (CAT) | extraction, | | substrates, | |
| | fluorescent, or | | time- | |
| | immunoassay | | consuming, | |
| | | | insensitive, | |
| | | | narrow linear | |
| | | | range | |
| β-galacto-sidase | colorimetric, | colorimetric | sensitive, | (Alam and |
| | fluorescence, | (histochemical | broad linear | Cook, 1990) |
| | chemiluminescence | staining with X- | range; some | |
| | | gal), bioluminescent | cells have high | |
| | | in | endogenous | |
| | | live cells | activity | |
| Secrete alkaline | colorimetric, | none | Chemiluminescence | (Berger et al., |
| phosphatase | bioluminescent, | | assay is | 1988) |
| (SEAP) | chemiluminescent | | sensitive and | |
| | | | broad linear | |
| | | | range; some | |
| | | | cells have | |
| | | | endogenous | |
| | | | alkaline | |
| | | | phosphatase | |
| | | | activity | |

Therapeutic Applications of DFF

Agonists and Antagonists

“Antagonist” includes any molecule that partially or fully blocks, inhibits, or neutralizes a biological activity of an endogenous DFF. Similarly, “agonist” includes any molecule that mimics a biological activity of an endogenous DFF. Molecules that can act as agonists or antagonists include Abs or antibody fragments, fragments or variants of endogenous DFF, peptides, antisense oligonucleotides, small organic molecules, etc.

Identifying Antagonists and Agonists

To assay for antagonists, a DFF is added to, or expressed in, a cell along with the compound to be screened for a particular activity. If the compound inhibits the activity of interest in the presence of the DFF, that compound is an antagonist to the DFF; if DFF activity is enhanced, the compound is an agonist. For example, a GLK antagonist inhibits GLK kinase activity; an agonist increases GLK kinase activity.

DFF-expressing cells are easily identified using standard methods. For example, antibodies that recognize the amino- or carboxy-terminus of a DFF can be used to screen candidate cells by immunoprecipitation, Western blots, and immunohistochemical techniques. Likewise, SEQ ID NOS: 1, 6 and 11 can be used to design primers and probes that detect a DFF mRNA in cells or samples from cells.

(a) Examples of Potential Antagonists and Agonist

Examples of antagonists and agonists include: (1) small organic and inorganic compounds, (2) small peptides, (3) Abs and derivatives, (4) polypeptides closely related to DFF, (5) antisense DNA and RNA, (6) ribozymes, (7) triple DNA helices and (8) nucleic acid aptamers.

Small molecules that bind to the DFF active site or other relevant part of the polypeptide and inhibit the biological activity of a DFF are antagonists. Examples of small molecule antagonists include small peptides, peptide-like molecules, preferably soluble, and synthetic non-peptidyl organic or inorganic compounds. These same molecules, if they enhance DFF activity, are examples of agonists.

Almost any antibody that affects a DFF function is a candidate antagonist, and occasionally, agonist. Examples of antibody antagonists include polyclonal, monoclonal, single-chain, anti-idiotypic, chimeric Abs, or humanized versions of such Abs or fragments. Abs may be from any species in which an immune response can be raised. Humanized Abs are also contemplated.

Alternatively, a potential antagonist or agonist may be a closely related polypeptide, for example, a mutated form of the DFF that recognizes a DFF-interacting polypeptide but imparts no effect other than competitively inhibiting DFF action. Alternatively, a mutated DFF can be constitutively activated and act as an agonist.

Antisense RNA or DNA constructs can be effective antagonists. Antisense RNA or DNA molecules block function by inhibiting translation by hybridizing to targeted mRNA. Antisense technology can be used to control gene expression through triple-helix formation or antisense DNA or RNA, both of which depend on polynucleotide binding to DNA or RNA. For example, the 5′ coding portion of a DFF sequence is used to design an antisense RNA oligonucleotide of from about 10 to 40 base pairs in length. A DNA oligonucleotide is designed to be complementary to a region of the gene involved in transcription (triple helix) (Beal and Dervan, 1991; Cooney et al., 1988; Lee et al., 1979), thereby preventing transcription and the production of a DFF. The antisense RNA oligonucleotide hybridizes to the mRNA in vivo and blocks translation of the mRNA molecule into a DFF (antisense) (Cohen, 1989; Okano et al., 1991). These oligonucleotides can also be delivered to cells such that the antisense RNA or DNA may be expressed in vivo to inhibit production of a DFF. When antisense DNA is used, oligodeoxyribonucleotides derived from the translation-initiation site, e.g., between about −10 and +10 positions of the target gene nucleotide sequence, are preferred.

To inhibit transcription, triple-helix nucleic acids that are single-stranded and comprise deoxynucleotides are useful antagonists. These oligonucleotides are designed such that triple-helix formation via Hoogsteen base-pairing rules is promoted, generally requiring stretches of purines or pyrimidines (WO 97/33551, 1997).

Aptamers are short oligonucleotide sequences that recognize and specifically bind almost any type of molecule. The systematic evolution of ligands by exponential enrichment (SELEX) process (Ausubel et al., 1987; Ellington and Szostak, 1990; Tuerk and Gold, 1990) is a powerful technique to identify aptamers. Aptamers have many diagnostic and clinical uses; almost any use in which an antibody is useful clinically or diagnostically, aptamers too may be used. Aptamers can be easily applied to a variety of formats, including administration in pharmaceutical compositions, in bioassays, and diagnostic tests (Jayasena, 1999).

Anti-DFF Abs

The invention encompasses Abs and antibody fragments, such as F_{ab} or (F_{ab})₂, that bind immunospecifically to any epitope of a DFF molecule.

“Antibody” (Ab) comprises single Abs directed against a DFF (an anti-DFF Ab; including agonist, antagonist, and neutralizing Abs), anti-DFF Ab compositions with poly-epitope specificity, single chain anti-DFF Abs, and fragments of anti-DFF Abs. A “monoclonal antibody” is obtained from a population of substantially homogeneous Abs, i.e., the individual Abs comprising the population are identical except for possible naturally-occurring mutations that may be present in minor amounts. Exemplary Abs include polyclonal (pAb), monoclonal (mAb), humanized, bi-specific (bsAb), and heteroconjugate Abs.

Polyclonal Abs (pAbs)

Polyclonal Abs can be raised in a mammalian host by one or more injections of an immunogen and, if desired, an adjuvant. Typically, the immunogen (and adjuvant) is injected in the mammal by multiple subcutaneous or intraperitoneal injections. The immunogen may include a DFF or a DFF fusion polypeptide. Examples of adjuvants include Freund's complete and monophosphoryl Lipid A synthetic-trehalose dicorynomycolate (MPL-TDM). To improve the immune response, an immunogen may be conjugated to a polypeptide that is immunogenic in the host, such as keyhole limpet hemocyanin (KLH), serum albumin, bovine thyroglobulin, and soybean trypsin inhibitor. Protocols for antibody production are well-known (Ausubel et al., 1987; Harlow and Lane, 1988). Alternatively, pAbs may be made in chickens, producing IgY molecules (Schade et al., 1996).

Monoclonal Abs (mAbs)

Anti-DFF mAbs may be prepared using hybridoma methods (Milstein and Cuello, 1983). Hybridoma methods comprise at least four steps: (1) immunizing a host, or lymphocytes from a host; (2) harvesting the mAb secreting (or potentially secreting) lymphocytes, (3) fusing the lymphocytes to immortalized cells, and (4) selecting those cells that secrete the desired (anti-DFF) mAb.

A mouse, rat, guinea pig, hamster, or other appropriate host is immunized to elicit lymphocytes that produce or are capable of producing Abs that will specifically bind to the immunogen. Alternatively, the lymphocytes may be immunized in vitro. If human cells are desired, peripheral blood lymphocytes (PBLs) are generally used; however, spleen cells or lymphocytes from other mammalian sources are preferred. The immunogen typically includes a DFF or a DFF fusion polypeptide.

The lymphocytes are then fused with an immortalized cell line to form hybridoma cells, facilitated by a fusing agent such as polyethylene glycol (Goding, 1996). Rodent, bovine, or human myeloma cells immortalized by transformation may be used, or rat or mouse myeloma cell lines. Because pure populations of hybridoma cells and not unfused immortalized cells are preferred, after fusion, the cells are grown in a suitable medium that inhibits the growth or survival of unfused, immortalized cells. A common technique uses parental cells that lack the enzyme hypoxanthine guanine phosphoribosyl transferase (HGPRT or HPRT). In this case, hypoxanthine, aminopterin and thymidine are added to the medium (HAT medium) to prevent the growth of HGPRT-deficient cells while permitting hybridomas to grow.

Preferred immortalized cells fuse efficiently; can be isolated from mixed populations by selecting in a medium such as HAT; and support stable and high-level expression of antibody after fusion. Preferred immortalized cell lines are murine myeloma lines, available from the American Type Culture Collection (Manassas, Va.). Human myeloma and mouse-human heteromyeloma cell lines also have been described for the production of human mAbs (Kozbor et al., 1984; Schook, 1987).

Because hybridoma cells secrete antibody extracellularly, the culture media can be assayed for the presence of mAbs directed against a DFF (anti-DFF mAbs). Immunoprecipitation or in vitro binding assays, such as radio immunoassay (RIA) or enzyme-linked immunoabsorbent assay (ELISA), measure the binding specificity of mAbs (Harlow and Lane, 1988; Harlow and Lane, 1999), including Scatchard analysis (Munson and Rodbard, 1980).

Anti-DFF mAb secreting hybridoma cells may be isolated as single clones by limiting dilution procedures and sub-cultured (Goding, 1996). Suitable culture media include Dulbecco's Modified Eagle's Medium, RPMI-1640, or if desired, a polypeptide-free or -reduced or serum-free medium (e.g., Ultra DOMA PF or HL-1; Biowhittaker; Walkersville, Md.). The hybridoma cells may also be grown in vivo as ascites.

The mAbs may be isolated or purified from the culture medium or ascites fluid by conventional Ig purification procedures such as polypeptide A-Sepharose, hydroxylapatite chromatography, gel electrophoresis, dialysis, ammonium sulfate precipitation or affinity chromatography (Harlow and Lane, 1988; Harlow and Lane, 1999).

The mAbs may also be made by recombinant methods (U.S. Pat. No. 4,166,452, 1979). DNA encoding anti-DFF mAbs can be readily isolated and sequenced using conventional procedures, e.g., using oligonucleotide probes that specifically bind to murine heavy and light antibody chain genes, to probe preferably DNA isolated from anti-DFF-secreting mAb hybridoma cell lines. Once isolated, the isolated DNA fragments are sub-cloned into expression vectors that are then transfected into host cells such as simian COS-7 cells, Chinese hamster ovary (CHO) cells, or myeloma cells that do not otherwise produce Ig polypeptide, to express mAbs. The isolated DNA fragments can be modified by substituting the coding sequence for human heavy and light chain constant domains in place of the homologous murine sequences (U.S. Pat. No. 4,816,567, 1989; Morrison et al., 1987), or by fusing the Ig coding sequence to all or part of the coding sequence for a non-Ig polypeptide. Such a non-Ig polypeptide can be substituted for the constant domains of an antibody, or can be substituted for the variable domains of one antigen-combining site to create a chimeric bivalent antibody.

Monovalent Abs

The Abs may be monovalent Abs that consequently do not cross-link each other. One method involves recombinant expression of Ig light chain and modified heavy chain. Heavy chain truncations generally at any point in the F_{c} region will prevent heavy chain cross-linking. Alternatively, the relevant cysteine residues are substituted with another amino acid residue or are deleted, preventing crosslinking by disulfide binding. In vitro methods are also suitable for preparing monovalent Abs. Abs can be digested to produce fragments, such as F_{ab} (Harlow and Lane, 1988; Harlow and Lane, 1999).

Humanized and Human Abs

Humanized forms of non-human Abs that bind a DFF are chimeric Igs, Ig chains or fragments (such as Fᵥ, F_{ab}, F_{ab′}, F_{(ab′)2} or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” Abs are chimeric Abs (U.S. Pat. No. 4,816,567, 1989), wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fᵥ framework residues of the human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (F_{c}), typically that of a human Ig (Jones et al., 1986; Presta, 1992; Riechmann et al., 1988).

Human Abs can also be produced using various techniques, including phage display libraries (Hoogenboom et al., 1991; Marks et al., 1991) and human mAbs (Boerner et al., 1991; Reisfeld and Sell, 1985). Introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire (U.S. Pat. Nos. 5,545,807, 1996; 5,569,825, 1996; 5,633,425, 1997; 5,661,016, 1997; 5,625,126, 1997; Fishwild et al., 1996; Lonberg and Huszar, 1995; Lonberg et al., 1994; Marks et al., 1992).

Bi-Specific mAbs

Bi-specific Abs are monoclonal, preferably human or humanized, that have binding specificities for at least two different antigens. For example, a binding specificity is a DFF; the other is for any antigen of choice, preferably a cell-surface polypeptide or receptor or receptor subunit.

The recombinant production of bi-specific Abs is often achieved byco-expressing two Ig heavy-chain/light-chain pairs, each having different specificities (Milstein and Cuello, 1983). The random assortment of these Ig heavy and light chains in the resulting hybridomas (quadromas) produce a potential mixture of ten different antibody molecules, of which only one has the desired bi-specific structure. The desired antibody can be purified using affinity chromatography or other techniques (WO 93/08829, 1993; Traunecker et al., 1991).

To manufacture a bi-specific antibody (Suresh et al., 1986), variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. DNAs encoding the Ig heavy-chain fusions and, if desired, the Ig light chain, are inserted into separate expression vectors and are co-transfected into a suitable host organism.

The interface between a pair of antibody molecules can be engineered to maximize the percentage of heterodimers that are recovered from recombinant cell culture (WO 96/27011, 1996). The preferred interface comprises at least part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This mechanism increases the yield of the heterodimer over unwanted end products such as homodimers.

Bi-specific Abs can be prepared as full length Abs or antibody fragments (e.g. F_{(ab′)2} bi-specific Abs). One technique to generate bi-specific Abs exploits chemical linkage. Intact Abs can be proteolytically cleaved to generate F_{(ab′)2} fragments (Brennan et al., 1985). Fragments are reduced with a dithiol complexing agent, such as sodium arsenite, to stabilize vicinal dithiols and prevent intermolecular disulfide formation. The generated F_{ab′} fragments are then converted to thionitrobenzoate (TNB) derivatives. One of the F_{ab′}-TNB derivatives is then reconverted to the F_{ab′}-thiol by reduction with mercaptoethylamine and is mixed with an equimolar amount of the other F_{ab′}-TNB derivative to form the bi-specific antibody. The produced bi-specific Abs can be used as agents for the selective immobilization of enzymes.

F_{ab′} fragments may be directly recovered from E. coli and chemically coupled to form bi-specific Abs. For example, fully humanized bi-specific F_{(ab′)2} Abs can be produced (Shalaby et al., 1992). Each F_{ab′} fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited (Kostelny et al., 1992). Peptides from the Fos and Jun polypeptides are linked to the F_{ab′} portions of two different Abs by gene fusion. The antibody homodimers are reduced at the hinge region to form monomers and then re-oxidized to form antibody heterodimers. This method can also produce antibody homodimers. “Diabody” technology (Holliger et al., 1993) provides an alternative method to generate bi-specific antibody fragments. The fragments comprise a heavy-chain variable domain (V_{H}) connected to a light-chain variable domain (V_{L}) by a linker that is too short to allow pairing between the two domains on the same chain. The V_{H} and V_{L} domains of one fragment are forced to pair with the complementary V_{L} and V_{H} domains of another fragment, forming two antigen-binding sites. Another strategy for making bi-specific antibody fragments is the use of single-chain Fᵥ (sFᵥ) dimers (Gruber et al., 1994). Abs with more than two valencies may also be made, such as tri-specific Abs (Tutt et al., 1991).

Exemplary bi-specific Abs may bind to two different epitopes on a given DFF. Alternatively, cellular defense mechanisms can be restricted to a particular cell expressing the particular DFF: an anti-DFF arm may be combined with an arm that binds to a leukocyte triggering molecule, such as a T-cell receptor molecule (e.g. CD2, CD3, CD28, or B7), or to F_{c} receptors for IgG (F_{c}γR), such as F_{c}γRI (CD64), F_{c}γRII (CD32) and F_{c}γRIII (CD16). Bi-specific Abs may also be used to target cytotoxic agents to cells that express a particular DFF. These Abs possess a DFF-binding arm and an arm that binds a cytotoxic agent or a radionuclide chelator.

Heteroconjugate Abs

Heteroconjugate Abs, consisting of two covalently joined Abs, target immune system cells to dispose unwanted cells (U.S. Pat. No. 4,676,980, 1987) and for treatment of human immunodeficiency virus (HIV) infection (WO 91/00360, 1991; WO 92/20373, 1992). Abs prepared in vitro using synthetic polypeptide chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate (U.S. Pat. No. 4,676,980, 1987).

Immunoconjugates

Immunoconjugates may comprise an antibody conjugated to a cytotoxic agent such as a chemotherapeutic agent, toxin (e.g., an enzymatically active toxin or fragment of bacterial, fungal, plant, or animal origin), or a radioactive isotope (i.e., a radioconjugate).

Useful enzymatically-active toxins and fragments include Diphtheria A chain, non-binding active fragments of Diphtheria toxin, exotoxin A chain from Pseudomonas aeruginosa, ricin A chain, abrin A chain, modeccin A chain, α-sarcin, Aleurites fordii polypeptides, Dianthin polypeptides, Phytolaca americana polypeptides, Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated Abs, such as ²¹²Bi, ¹³¹I, ¹³¹In, ⁹⁰Y, and ¹⁸⁶Re.

Conjugates of the antibody and cytotoxic agent are made using a variety of bi-functional polypeptide-coupling agents, such as N-succinimidyl-3-(2-pyridyldithiol) propionate (SPDP), iminothiolane (IT), bi-functional derivatives of imidoesters (such as dimethyl adipimidate HCl), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared (Vitetta et al., 1987). ¹⁴C-labeled 1-isothiocyanatobenzyl-3-methyldiethylene triaminepentaacetic acid (MX-DTPA) is an exemplary chelating agent for conjugating radionuclide to antibody (WO 94/11026, 1994).

The antibody may be conjugated to a “receptor” (such as streptavidin) to use in tumor pre-targeting, wherein the antibody-receptor conjugate is administered to the patient, followed by removal of unbound conjugate from the circulation using a clearing agent and then administration of a streptavidin “ligand” (e.g., biotin) that is conjugated to a cytotoxic agent (e.g., a radionuclide).

Effector Function Engineering

Antibodies can be modified to enhance their effectiveness in treating a disease, such as obesity, to target and kill adipose cells. For example, cysteine residue(s) may be introduced into the F_{c} region, thereby allowing interchain disulfide bond formation in this region. Such homodimeric Abs often have improved internalization capability and/or increased complement-mediated cell killing and antibody-dependent cellular cytotoxicity (ADCC) (Caron et al., 1992; Shopes, 1992). Homodimeric Abs with enhanced activity can be prepared using hetero-bifunctional cross-linkers (Wolff et al., 1993). Alternatively, an antibody engineered with dual F_{c} regions may have enhanced complement lysis (Stevenson et al., 1989).

Immunoliposomes

Liposomes containing the antibody (immunoliposomes) may also be formulated (U.S. Pat. Nos. 4,485,045, 1984; 4,544,545, 1985; 5,013,556, 1991; Eppstein et al., 1985; Hwang et al., 1980). Useful liposomes can be generated by a reverse-phase evaporation method with a lipid composition comprising phosphatidylcholine, cholesterol, and PEG-derivatized phosphatidylethanolamine (PEG- PE). Such preparations are extruded through filters of defined pore size to yield liposomes with a desired diameter. F_{ab′} fragments of the antibody can be conjugated to the liposomes (Martin and Papahadjopoulos, 1982) via a disulfide-interchange reaction. A chemotherapeutic agent, such as Doxorubicin, may also be contained in the liposome (Gabizon et al., 1989). Other useful liposomes with different compositions are contemplated.

Diagnostic Applications of Abs Directed Against DFF

Anti-DFF Abs can be used to localize and/or quantitate DFF (e.g., for use in measuring levels of DFF within tissue samples or for use in diagnostic methods, etc.). Anti-DFF epitope Abs can be utilized as pharmacologically active compounds.

Anti-DFF Abs can be used to isolate a specific DFF by standard techniques, such as immunoaffinity chromatography or immunoprecipitation. These approaches facilitate purifying endogenous DFF antigen-containing polypeptides from cells and tissues. Such approaches can be used to detect DFF in a sample to evaluate the abundance and pattern of expression of the antigenic polypeptide. Anti-DFF Abs can be used to monitor polypeptide levels in tissues as part of a clinical testing procedure; for example, to determine the efficacy of a given treatment regimen. Coupling the antibody to a detectable substance (label) allows detection of Ab-antigen complexes. Classes of labels include fluorescent, luminescent, bioluminescent, and radioactive materials, enzymes and prosthetic groups. Useful labels include horseradish peroxidase, alkaline phosphatase, β-galactosidase, acetylcholinesterase, streptavidin/biotin, avidin/biotin, umbelliferone, fluorescein, fluorescein isothiocyanate, rhodamine, dichlorotriazinylamine fluorescein, dansyl chloride, phycoerythrin, luminol, luciferase, luciferin, aequorin, and ¹²⁵I, ¹³¹I, ³⁵S or ³H.

Antibody Therapeutics

Abs can be used therapeutically to treat or prevent a disease or pathology in a subject. An antibody preparation, preferably one having high antigen specificity and affinity, generally mediates an effect by binding the target epitope(s). Administration of such Abs may mediate one of two effects: (1) the antibody may prevent ligand binding, eliminating endogenous ligand binding and subsequent signal transduction, or (2) the antibody elicits a physiological response by binding an effector site on the target molecule, initiating signaling.

A therapeutically effective amount of an antibody relates generally to the amount needed to achieve a therapeutic objective, epitope binding affinity, administration rate, and depletion rate of the antibody from a subject. Common ranges for therapeutically effective doses are about 0.1 mg/kg body weight to about 50 mg/kg body weight. Dosing frequencies may range, for example, from twice daily to once a week.

Pharmaceutical Compositions of Abs

Anti-DFF Abs, as well as other DFF interacting molecules (such as aptamers) identified in other assays, can be administered in pharmaceutical compositions to treat various disorders. Principles and considerations involved in preparing such compositions, as well as guidance in the choice of components are well described (de Boer, 1994; Gennaro, 2000; Lee, 1990).

Abs that are internalized are preferred when whole Abs are used as inhibitors and the target is intracellular. Liposomes can be used to deliver intracellularly. Where antibody fragments are used, the smallest inhibitory fragment that specifically binds to the epitope is preferred. For example, peptide molecules can be designed that bind a preferred epitope based on the variable-region sequences of a useful antibody. Such peptides can be synthesized chemically and/or produced by recombinant DNA technology (Marasco et al., 1993). Formulations may also contain more than one active compound for a particular treatment, preferably those with activities that do not adversely affect each other. The composition may comprise an agent that enhances function, such as a cytotoxic agent, cytokine, chemotherapeutic agent, or growth-inhibitory agent.

The active ingredients can also be entrapped in microcapsules prepared by coacervation techniques or by interfacial polymerization; for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration are highly preferred to be sterile. This is readily accomplished by filtration through sterile filtration membranes or any of a number of techniques.

Sustained-release preparations may also be prepared, such as semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (Boswell and Scribner, U.S. Pat. No. 3,773,919, 1973), copolymers of L-glutamic acid and γ ethyl-L-glutamate, non-degradable ethylene-vinyl acetate, degradable lactic acid-glycolic acid copolymers such as injectable microspheres composed of lactic acid-glycolic acid copolymer, and poly-D-(−)-3-hydroxybutyric acid. While polymers such as ethylene-vinyl acetate and lactic acid-glycolic acid enable release of molecules for over 100 days, certain hydrogens release polypeptides for shorter time periods and may be preferred.

DFF Recombinant Expression Vectors and Host Cells

Vectors are tools used to shuttle DNA between host cells or as a means to express a nucleotide sequence. Some vectors function only in prokaryotes, while others function in both prokaryotes and eukaryotes, enabling large-scale DNA preparation from prokaryotes for expression in eukaryotes. Inserting the DNA of interest, such as a DFF nucleotide sequence or a fragment, is accomplished by ligation techniques and/or mating protocols well known to the skilled artisan. Such DNA is inserted such that its integration does not disrupt any necessary components of the vector. In the case of vectors that are used to express the inserted DNA polypeptide, the introduced DNA is operably-linked to the vector elements that govern its transcription and translation.

Vectors can be divided into two general classes: Cloning vectors are replicating plasmid or phage with regions that are non-essential for propagation in an appropriate host cell, and into which foreign DNA can be inserted; the foreign DNA is replicated and propagated as if it were a component of the vector. An expression vector (such as a plasmid, yeast, or animal virus genome) is used to introduce foreign genetic material into a host cell or tissue in order to transcribe and translate the foreign DNA. In expression vectors, the introduced DNA is operably-linked to elements, such as promoters, that signal to the host cell to transcribe the inserted DNA. Some promoters are exceptionally useful, such as inducible promoters that control gene transcription in response to specific factors. Operably-linking a DFF or anti-sense construct to an inducible promoter can control the expression of a DFF or fragments, or anti-sense constructs. Examples of classic inducible promoters include those responsive to α-interferon, heat-shock, heavy metal ions, and steroids such as glucocorticoids (Kaufman, 1990) and tetracycline. Other desirable inducible promoters include those that are not endogenous to the cells in which the construct is being introduced, but, however, is responsive in those cells when the induction agent is exogenously supplied.

Vectors have many manifestations. A “plasmid” is a circular double stranded DNA molecule that can accept additional DNA fragments. Viral vectors can also accept additional DNA segments into the viral genome. Certain vectors are capable of autonomous replication in a host cell (e.g., bacterial vectors having a bacterial origin of replication and episomal mammalian vectors). Other vectors (e.g., non-episomal mammalian vectors) integrate into the genome of a host cell and replicate as part of the host genome. In general, useful expression vectors are plasmids and viral vectors (e.g., replication defective retroviruses, adenoviruses and adeno-associated viruses); other expression vectors can also be used.

Recombinant expression vectors that comprise a DFF (or fragment(s)) regulate a DFF transcription by exploiting one or more host cell-responsive (or that can be manipulated in vitro) regulatory sequences that is operably-linked to DFF. “Operably-linked” indicates that a nucleotide sequence of interest is linked to regulatory sequences such that expression of the nucleotide sequence is achieved.

Vectors can be introduced in a variety of organisms and/or cells (Table D). Alternatively, the vectors can be transcribed and translated in vitro, for example using T7 promoter regulatory sequences and T7 polymerase.

TABLE D
Examples of hosts for cloning or expression

| | | |
|---|---|---|
| Organisms | Examples | Sources and References* |

Prokaryotes

| | | |
|---|---|---|
| Enterobacteriaceae | E. coli | |
| | K 12 strain MM294 | ATCC 31,446 |
| | X1776 | ATCC 31,537 |
| | W3110 | ATCC 27,325 |
| | K5 772 | ATCC 53,635 |
| | Enterobacter | |
| | Erwinia | |
| | Klebsiella | |
| | Proteus | |
| | Salmonella | |
| | (S. tyhpimurium) | |
| | Serratia (S. marcescans) | |
| | Shigella | |
| | Bacilli (B. subtilis and B. | |
| | licheniformis) | |
| | Pseudomonas | |
| | (P. aeruginosa) | |
| | Streptomyces | |
| Eukaryotes | | |
| Yeasts | Saccharomyces | |
| | cerevisiae | |
| | Schizosaccharomyces | |
| | pombe | |
| | Kluyveromyces | (Fleer et al., 1991) |
| | K. lactis MW98-8C, | (de Louvencourt et al., |
| | CBS683, CBS4574 | 1983) |
| | K. fragilis | ATCC 12,424 |
| | K. bulgaricus | ATCC 16,045 |
| | K. wickeramii | ATCC 24,178 |
| | K. waltii | ATCC 56,500 |
| | K. drosophilarum | ATCC 36,906 |
| | K. thermotolerans | |
| | K. marxianus; yarrowia | (EPO 402226, 1990) |
| | Pichia pastoris | (Sreekrishna et al., 1988) |
| | Candida | |
| | Trichoderma reesia | |
| | Neurospora crassa | (Case et al., 1979) |
| | Torulopsis | |
| | Rhodotorula | |
| | Schwanniomyces (S. | |
| | occidentalis) | |
| Filamentous Fungi | Neurospora | |
| | Penicillium | |
| | Tolypocladium | (WO 91/00357, 1991) |
| | Aspergillus (A. nidulans | (Kelly and Hynes, 1985; |
| | and A.niger) | Tilburn et al., 1983; |
| | | Yelton et al., 1984) |
| Invertebrate cells | Drosophila S2 | |
| | Spodoptera Sf9 | |
| Vertebrate cells | Chinese Hamster Ovary | |
| | (CHO) | |
| | simian COS | ATCC CRL 1651 |
| | COS-7 | |
| | HEK 293 | |

*Unreferenced cells are generally available from American Type Culture Collection (Manassas, VA).

Vector choice is dictated by the organism or cells being used, and the desired fate of the vector. Vectors may replicate once in the target cells, or may be “suicide” vectors. In general, vectors comprise signal sequences, origins of replication, marker genes, enhancer elements, promoters, and transcription termination sequences. The choice of these elements depends on the organisms in which the vector will be used. Some of these elements may be conditional, such as an inducible or conditional promoter that is turned “on” when conditions are appropriate. Examples of inducible promoters include those that are tissue-specific, which relegate expression to certain cell types, steroid-responsive, or heat-shock reactive. Some bacterial repression systems, such as the lac operon, have been exploited in mammalian cells and transgenic animals (Fieck et al., 1992; Wyborski et al., 1996; Wyborski and Short, 1991). Vectors often use a selectable marker to facilitate identifying those cells that have incorporated the vector. Many selectable markers are well known in the art for the use with prokaryotes, usually antibiotic-resistance genes or the use of autotrophy and auxotrophy mutants. Table F summarizes many of the available markers.

“Host cell” and “recombinant host cell” are used interchangeably. Such terms refer not only to a particular subject cell but also to the progeny or potential progeny of such a cell. Because certain modifications may occur in succeeding generations due to either mutation or environmental influences, such progeny may not, in fact, be identical to the parent cell, but are still included within the scope of the term.

Methods of eukaryotic cell transfection and prokaryotic cell transformation are well known in the art. The choice of host cell dictates the preferred technique for introducing the nucleic acid of interest. Table E summarizes many known techniques in the art. Introduction of nucleic acids into an organism may also be done with ex vivo techniques that use an in vitro method of transfection, as well as established genetic techniques, if any, for that particular organism.

TABLE E
Methods to introduce polynucleotide into cells

| Cells | Methods | References | Notes |
|---|---|---|---|
| Prokaryotes | Calcium chloride | (Cohen et al., 1972; Hanahan, 1983; Mandel and | |
| (bacteria) | | Higa, 1970) | |
| | Electroporation | (Shigekawa and Dower, 1988) | |
| Eukaryotes | Calcium phosphate | N-(2-Hydroxyethyl)piperazine-N′-(2-ethanesulfonic | Cells may be “shocked” |
| Mammalian cells | transfection | acid (HEPES) buffered saline solution (Chen and | with glycerol or |
| | | Okayama, 1988; Graham and van der Eb, 1973; | dimethylsulfoxide (DMSO) |
| | | Wigler et al., 1978) | to increase transfection |
| | | BES (N,N-bis(2-hydroxyethyl)-2-aminoethanesulfonic | efficiency (Ausubel et al., |
| | | acid) buffered solution (Ishiura et al., 1982) | 1987). |
| | Diethylaminoethyl | (Fujita et al., 1986; Lopata et al., 1984; Selden et al., | Most useful for transient, |
| | (DEAE)-Dextran | 1986) | but not stable, |
| | transfection | | transfections. |
| | | | Chloroquine can be used to |
| | | | increase efficiency. |
| | Electroporation | (Neumann et al., 1982; Potter, 1988; Potter et al., | Especially useful for hard- |
| | | 1984; Wong and Neumann, 1982) | to-transfect lymphocytes. |
| | Cationic lipid reagent | (Elroy-Stein and Moss, 1990; Felgner et al., 1987; | Applicable to both in vivo |
| | transfection | Rose et al., 1991; Whitt et al., 1990) | and in vitro transfection. |
| | Retroviral | Production exemplified by (Cepko et al., 1984; Miller | Lengthy process, many |
| | | and Buttimore, 1986; Pear et al., 1993) | packaging lines available at |
| | | Infection in vitro and in vivo: (Austin and Cepko, | ATCC. Applicable to both |
| | | 1990; Bodine et al., 1991; Fekete and Cepko, 1993; | in vivo and in vitro |
| | | Lemischka et al., 1986; Turner et al., 1990; Williams | transfection. |
| | | et al., 1984) | |
| | Polybrene | (Chaney et al., 1986; Kawai and Nishizawa, 1984) | |
| | Microinjection | (Capecchi, 1980) | Can be used to establish |
| | | | cell lines carrying |
| | | | integrated copies of DFF |
| | | | DNA sequences. |
| | Protoplast fusion | (Rassoulzadegan et al., 1982; Sandri-Goldin et al., | |
| | | 1981; Schaffner, 1980) | |
| Insect cells (in | Baculovirus systems | (Luckow, 1991; Miller, 1988; O'Reilly et al., 1992) | Useful for in vitro |
| vitro) | | | production of polypeptides |
| | | | with eukaryotic |
| | | | modifications. |
| Yeast | Electroporation | (Becker and Guarente, 1991) | |
| | Lithium acetate | (Gietz et al., 1998; Ito et al., 1983) | |
| | Spheroplast fusion | (Beggs, 1978; Hinnen et al., 1978) | Laborious, can produce |
| | | | aneuploids. |
| Plant cells | Agrobacterium | (Bechtold and Pelletier, 1998; Escudero and Hohn, | |
| (general reference: | transformation | 1997; Hansen and Chilton, 1999; Touraev and al., | |
| (Hansen and | | 1997) | |
| Wright, 1999)) | Biolistics | (Finer et al., 1999; Hansen and Chilton, 1999; | |
| | (microprojectiles) | Shillito, 1999) | |
| | Electroporation | (Fromm et al., 1985; Ou-Lee et al., 1986; Rhodes et | |
| | (protoplasts) | al., 1988; Saunders et al., 1989) | |
| | | May be combined with liposomes (Trick and al., | |
| | | 1997) | |
| | Polyethylene glycol | (Shillito, 1999) | |
| | (PEG) treatment | | |
| | Liposomes | May be combined with electroporation (Trick and al., | |
| | | 1997) | |
| | in planta microinjection | (Leduc and al., 1996; Zhou and al., 1983) | |
| | Seed imbibition | (Trick and al., 1997) | |
| | Laser beam | (Hoffman, 1996) | |
| | Silicon carbide whiskers | (Thompson and al., 1995) | |

TABLE F
Useful selectable markers for eukaryote cell transfection

| Selectable Marker | Selection | Action | Reference |
|---|---|---|---|
| Adenosine deaminase (ADA) | Media includes 9-β-D-xylofuranosyl | Conversion of Xyl-A to Xyl-ATP, which | (Kaufman et |
| | adenine (Xyl-A) | incorporates into polynucleotides, killing | al., 1986) |
| | | cells. ADA detoxifies | |
| Dihydrofolate reductase | Methotrexate (MTX) and dialyzed | MTX competitive inhibitor of DHFR. In | (Simonsen |
| (DHFR) | serum (purine-free media) | absence of exogenous purines, cells require | and |
| | | DHFR, a necessary enzyme in purine | Levinson, |
| | | biosynthesis. | 1983) |
| Aminoglycoside | G418 | G418, an aminoglycoside detoxified by | (Southern |
| phosphotransferase (“APH”, | | APH, interferes with ribosomal function and | and Berg, |
| “neo”, “G418”) | | consequently, translation. | 1982) |
| Hygromycin-B- | hygromycin-B | Hygromycin-B, an aminocyclitol detoxified | (Palmer et |
| phosphotransferase (HPH) | | by HPH, disrupts polypeptide translocation | al., 1987) |
| | | and promotes mistranslation. | |
| Thymidine kinase (TK) | Forward selection (TK+): Media | Forward: Aminopterin forces cells to | (Littlefield, |
| | (HAT) incorporates aminopterin. | synthesize dTTP from thymidine, a pathway | 1964) |
| | Reverse selection (TK−): Media | requiring TK. | |
| | incorporates 5-bromodeoxyuridine | Reverse: TK phosphorylates BrdU, which | |
| | (BrdU). | incorporates into polynucleotides, killing | |
| | | cells. | |

A host cell, prokaryotic or eukaryotic, can be used to produce a DFF in culture. To accomplish in vitro expression of a DFF, a host cell containing a recombinant expression vector encoding a DFF is expressed, when cultured in a suitable medium. The DFF may then be isolated from the media or culture.

Transgenic DFF Animals

Transgenic animals are useful for studying the function and/or activity of a DFF and for identifying and/or evaluating modulators of a DFF activity. “Transgenic animals” are non-human animals, preferably mammals, more preferably rodents such as rats or mice, in which one or more of the cells include a transgene. Other transgenic animals include primates, sheep, dogs, cows, goats, chickens, amphibians, etc. A “transgene” is exogenous DNA that is integrated into the genome of a cell from which a transgenic animal develops and that remains in the genome of the mature animal. Transgenes preferably direct the expression of an encoded gene product in one or more cell types or tissues, preventing expression of a naturally encoded gene product in one or more cell types or tissues (a “knockout” transgenic animal), over-expressing an encoded gene, or serving as a marker or indicator of an integration, chromosomal location, or region of recombination (e.g. cre/loxP mice). A “homologous recombinant animal” is a non-human animal, such as a rodent, in which an endogenous DFF has been altered by an exogenous DNA molecule that recombines homologously with an endogenous DFF in a (e.g. embryonic) cell prior to development the animal. Host cells with an exogenous DFF can be used to produce non-human transgenic animals, such as fertilized oocytes or embryonic stem cells into which a DFF coding sequence has been introduced. Such host cells can then be used to create non-human transgenic animals or homologous recombinant animals.

Approaches to Transgenic Animal Production

A transgenic animal can be created by introducing a DFF into the male pronuclei of a fertilized oocyte (e.g., by microinjection, retroviral infection, etc.) and allowing the oocyte to develop in a pseudopregnant female foster animal (pffa). The DFF sequences (SEQ ID NOs: 1, 6 or 11) can be introduced as a transgene into the genome of a non-human animal. Alternatively, a homologue of a DFF can be used as a transgene. Intronic sequences and polyadenylation signals can also be included in the transgene to increase transgene expression. Tissue-specific regulatory sequences can be operably-linked to the DFF transgene to direct expression of DFF to particular cells. Methods for generating transgenic animals via embryo manipulation and microinjection, particularly animals such as mice, have become conventional in the art, e.g. (Evans et al., U.S. Pat. No. 4,870,009, 1989; Hogan, 0879693843, 1994; Leder and Stewart, U.S. Pat. No. 4,736,866, 1988; Wagner and Hoppe, U.S. Pat. No. 4,873,191, 1989). Other non-mice transgenic animals may be made by similar methods. A transgenic founder animal, which can be used to breed additional transgenic animals, can be identified based upon the presence of the transgene in its genome and/or expression of the transgene mRNA in tissues or cells of the animals. Transgenic (e.g. DFF) animals can be bred to other transgenic animals carrying other transgenes.

Vectors for Transgenic Animal Production

To create a homologous recombinant animal, a vector containing at least a portion of a DFF into which a deletion, addition or substitution has been introduced to thereby alter, e.g., functionally disrupt, the DFF. The DFF can be a mouse gene (SEQ ID NOS: 1, 5 or 11), or a DFF homologue. In one approach, a knockout vector functionally disrupts an endogenous DFF gene upon homologous recombination, and thus a non-functional DFF polypeptide, if any, is expressed.

Alternatively, the vector can be designed such that, upon homologous recombination, an endogenous DFF is mutated or otherwise altered but still encodes functional polypeptide (e.g., the upstream regulatory region can be altered to thereby alter the expression of an endogenous DFF). In this type of homologous recombination vector, the altered portion of a DFF is flanked at its 5′- and 3′-termini by additional nucleic acid of a DFF to allow for homologous recombination to occur between the exogenous DFF carried by the vector and an endogenous DFF in an embryonic stem cell. The additional flanking DFF nucleic acid is sufficient to engender homologous recombination with the target endogenous DFF. Typically, several kilobases of flanking DNA (both at the 5′- and 3′-termini) are included in the vector (Thomas and Capecchi, 1987). The vector is then introduced into an embryonic stem cell line, and cells in which the introduced DFF has homologously-recombined with an endogenous DFF are selected (Li et al., 1992).

Introduction of DFF Transgene Cells During Development

Selected cells are then injected into a blastocyst of an animal to form aggregation chimeras (Bradley, 1987). A chimeric embryo can then be implanted into a suitable pffa and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are well-described (Berns et al., WO 93/04169, 1993; Bradley, 1991; Kucherlapati et al., WO 91/01140, 1991; Le Mouellic and Brullet, WO 90/11354, 1990).

Alternatively, transgenic animals that contain selected systems that allow for regulated expression of the transgene can be produced. For example, the cre/loxP recombinase system of bacteriophage P1 (Lakso et al., 1992) or the FLP recombinase system of Saccharomyces cerevisiae (O'Gorman et al., 1991) may be used. In cre/loxP recombinase systems, animals containing transgenes encoding both the Cre recombinase and a selected polypeptide are required. Such animals can be produced as “double” transgenic animals, by mating an animal containing a transgene encoding a selected polypeptide to another containing a transgene encoding a recombinase.

Clones of transgenic animals can also be produced (Wilmut et al., 1997). In brief, a cell from a transgenic animal can be isolated and induced to exit the growth cycle and enter G₀ phase. The quiescent cell can then be fused to an enucleated oocyte from an animal of the same species from which the quiescent cell is isolated. The reconstructed oocyte is then cultured to develop to a morula or blastocyte and then transferred to a pffa. The offspring borne of this female foster animal will be a clone of the “parent” transgenic animal.

Pharmaceutical Compositions

The DFF nucleic acid molecules, DFF polypeptides, and anti-DFF Abs, and their derivatives, fragments, analogs and homologs, can be incorporated into pharmaceutical compositions. Such compositions typically comprise a nucleic acid molecule, polypeptide, or antibody and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (Gennaro, 2000). Preferred examples of such carriers or diluents include, but are not limited to, water, saline, finger's solutions, dextrose solution, and 5% human serum albumin. Liposomes and non-aqueous vehicles such as fixed oils may also be used. Except when a conventional media or agent is incompatible with an active compound, use of these compositions is contemplated. Supplementary active compounds can also be incorporated into the compositions.

General Considerations

A pharmaceutical composition is formulated to be compatible with the intended route of administration, including intravenous, intradermal, subcutaneous, oral (e.g., inhalation), transdermal (i.e., topical), transmucosal, and rectal administration. Solutions or suspensions used for parenteral, intradermal, or subcutaneous application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Injectable Formulations

To access adipose tissue, injection provides a direct and facile route, especially for that tissue that is below the skin. Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, the composition must be sterile and should be fluid so as to be administered using a syringe. Such compositions should be stable during manufacture and storage and must be preserved against contamination from microorganisms such as bacteria and fungi. The carrier can be a solvent or dispersion medium containing, for example, water, ethanol, polyol (such as glycerol, propylene glycol, and liquid polyethylene glycol), and suitable mixtures. Proper fluidity can be maintained, for example, by using a coating such as lecithin, by maintaining the required particle size in the case of dispersion and by using surfactants. Various antibacterial and antifungal agents, such as parabens, chlorobutanol, phenol, ascorbic acid, and thimerosal, can control microorganism contamination. Isotonic agents, such as sugars, polyalcohols such as manitol, sorbitol, and sodium chloride can be included in the composition. Compositions that delay absorption include agents such as aluminum monostearate and gelatin.

Sterile injectable solutions can be prepared by incorporating the active compound (e.g., a DFF or anti-DFF antibody) in an appropriate solvent with one or a combination of ingredients, followed by sterilization. Generally, dispersions are prepared by incorporating the active compound into a sterile vehicle that contains a basic dispersion medium, and any other required ingredients. Sterile powders for the preparation of sterile injectable solutions methods of preparation include vacuum drying and freeze-drying that yield a powder containing the active ingredient and any desired ingredient from a sterile solutions.

Oral Compositions

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally. Pharmaceutically compatible binding agents, and/or adjuvant materials can be included. Tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL, or corn starch; a lubricant such as magnesium stearate or STEROTES; a glidant such as colloidal silicon dioxide; a sweetening agent such as sucrose or saccharin; or a flavoring agent such as peppermint, methyl salicylate, or orange flavoring.

Compositions for Inhalation

For administration by inhalation, the compounds are delivered as an aerosol spray from a nebulizer or a pressurized container that contains a suitable propellant, e.g., a gas such as carbon dioxide.

Systemic Administration

Systemic administration can also be transmucosal or transdermal. For transmucosal or transdermal administration, penetrants that can permeate the target barrier(s) are selected. Transmucosal penetrants include, detergents, bile salts, and fusidic acid derivatives. Nasal sprays or suppositories can be used for transmucosal administration. For transdermal administration, the active compounds are formulated into ointments, salves, gels, or creams.

The compounds can also be prepared in the form of suppositories (e.g., with bases such as cocoa butter and other glycerides) or retention enemas for rectal delivery.

Carriers

In one embodiment, the active compounds are prepared with carriers that protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid (ALZA Corporation; Mountain View, Calif. and NOVA Pharmaceuticals, Inc.; Lake Elsinore, Calif.; or prepared by one of skill in the art). Liposomal suspensions can also be used as pharmaceutically acceptable carriers. These can be prepared according to methods known to those skilled in the art (Eppstein et al., U.S. Pat. No. 4,522,811, 1985).

Unit Dosage

Oral formulations or parenteral compositions in unit dosage form can be created to facilitate administration and dosage uniformity. Unit dosage form refers to physically discrete units suited as single dosages for a subject to be treated, containing a therapeutically effective quantity of active compound in association with the required pharmaceutical carrier. The specification for unit dosage forms are dictated by, and directly dependent on, the unique characteristics of the active compound and the particular desired therapeutic effect, and the inherent limitations of compounding the active compound

Gene Therapy Compositions

The nucleic acid molecules of the invention can be inserted into vectors and used as gene therapy vectors. Gene therapy vectors can be delivered to a subject by, for example, intravenous injection, local administration (Nabel and Nabel, U.S. Pat. No. 5,328,470, 1994), or by stereotactic injection (Chen et al., 1994). The pharmaceutical preparation of a gene therapy vector can include an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Dosage

The pharmaceutical compositions and methods of the present invention may further comprise other therapeutically active compounds that are usually applied in the treatment of adipose-related pathologies.

In the treatment or prevention of conditions which require modulation of a DFF, an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to a patient to be treated. The compounds may be administered on a regimen of 1 to 4 times per day, preferably once or twice per day.

It will be understood, however, that the specific dose level and frequency of dosage for any particular patient may be varied and depends upon a variety of factors, including the activity of the specific compound employed, the metabolic stability and length of action of that compound, the age, body weight, general health, sex, diet, mode and time of administration, rate of excretion, drug combination, the severity of the particular condition, and the host undergoing therapy.

Kits for Pharmaceutical Compositions

The pharmaceutical compositions can be included in a kit, container, pack, or dispenser together with instructions for administration. When the invention is supplied as a kit, the different components of the composition may be packaged in separate containers and admixed immediately before use. Such packaging of the components separately may permit long-term storage without losing the active components' functions.

Kits may also include reagents in separate containers that facilitate the execution of a specific test, such as diagnostic tests or tissue typing. For example, DFF DNA templates and suitable primers may be supplied for internal controls.

(a) Containers or Vessels

The reagents included in the kits can be supplied in containers of any sort such that the life of the different components are preserved, and are not adsorbed or altered by the materials of the container. For example, sealed glass ampules may contain lyophilized luciferase or buffer that have been packaged under a neutral, non-reacting gas, such as nitrogen. Ampules may consist of any suitable material, such as glass, organic polymers, such as polycarbonate, polystyrene, etc., ceramic, metal or any other material typically employed to hold reagents. Other examples of suitable containers include simple bottles that may be fabricated from similar substances as ampules, and envelopes, that may consist of foil-lined interiors, such as aluminum or an alloy. Other containers include test tubes, vials, flasks, bottles, syringes, or the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, etc.

(b) Instructional Materials

Kits may also be supplied with instructional materials. Instructions may be printed on paper or other substrate, and/or may be supplied as an electronic-readable medium, such as a floppy disc, CD-ROM, DVD-ROM, Zip disc, videotape, audio tape, etc. Detailed instructions may not be physically associated with the kit; instead, a user may be directed to an internet web site specified by the manufacturer or distributor of the kit, or supplied as electronic mail.

Screening and Detection Methods

The isolated nucleic acid molecules of the invention can be used to express a DFF (e.g., via a recombinant expression vector in a host cell in gene therapy applications), to detect a DFF mRNA (e.g., in a biological sample) or a genetic lesion in a DFF, and to modulate a DFF activity. In addition, DFF polypeptides can be used to screen drugs or compounds that modulate a DFF activity or expression as well as to treat disorders characterized by insufficient or excessive production of a DFF or production of forms of a DFF that have decreased or aberrant activity compared to DFF wild-type polypeptide, or modulate biological function that involve DFF. In addition, the anti-DFF Abs of the invention can be used to detect and isolate a DFF and modulate a DFF activity.

Screening Assays

The invention provides a method (screening assay) for identifying modalities, i.e., candidate or test compounds or agents (e.g., peptides, peptidomimetics, small molecules or other drugs), foods, combinations thereof, etc., that effect a DFF as a stimulatory or inhibitory effect, inlcuding translation, transcription, activity or copies of the gene in cells. The invention also includes compounds identified in screening assays.

Testing for compounds that increase or decrease DFF activity are desirable. A compound may modulate DFF activity by affecting: (1) the number of copies of the gene in the cell (amplifiers and deamplifiers); (2) increasing or decreasing transcription of the DFF (transcription up-regulators and down-regulators); (3) by increasing or decreasing the translation of DFF mRNA into polypeptide (translation up-regulators and down-regulators); or (4) by increasing or decreasing the activity of DFF itself (agonists and antagonists).

(a) Effects of Compounds

To identify compounds that affect a DFF at the DNA, RNA and polypeptide levels, cells or organisms are contacted with a candidate compound, and the corresponding change in the target DFF DNA, RNA or polypeptide is assessed (Ausubel et al., 1987). For DNA amplifiers and deamplifiers, the amount of a DFF DNA is measured; for those compounds that are transcription up-regulators and down-regulators, the amount of DFF mRNA is determined; for translational up- and down-regulators, the amount of DFF polypeptides is measured. Compounds that are agonists or antagonists may be identified by contacting cells or organisms with the compound.

Many assays for screening candidate or test compounds that bind to or modulate the activity of a DFF or DFF polypeptide or biologically active portion are available. Test compounds can be obtained using any of the numerous approaches in combinatorial library methods, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptides, while the other four approaches encompass peptide, non-peptide oligomer or small molecule libraries of compounds (Lam, 1997).

(b) Small Molecules

A “small molecule” refers to a composition that has a molecular weight of less than about 5 kD and more preferably less than about 4 kD, and most preferably less than 0.6 kD. Small molecules can be nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. Examples of methods for the synthesis of molecular libraries are described (Carell et al., 1994a; Carell et al., 1994b; Cho et al., 1993; DeWitt et al., 1993; Gallop et al., 1994; Zuckermann et al., 1994).

Libraries of compounds may be presented in solution (Houghten et al., 1992) or on beads (Lam et al., 1991), on chips (Fodor et al., 1993), bacteria, spores (Ladner et al., U.S. Pat. No. 5,223,409, 1993), plasmids (Cull et al., 1992) or phage (Cwirla et al., 1990; Devlin et al., 1990; Felici et al., 1991; Ladner et al., U.S. Pat. No. 5,223,409, 1993; Scott and Smith, 1990). A cell-free assay comprises contacting a DFF or biologically-active fragment with a known compound that binds a DFF to form an assay mixture, contacting the assay mixture with a test compound, and determining the ability of the test compound to interact with the target DFF, where determining the ability of the test compound to interact with the target DFF comprises determining the ability of the target DFF to preferentially bind to or modulate the activity of a DFF target molecule.

(c) Cell-Free Assays

The cell-free assays of the invention may be used with both soluble or a membrane-bound forms of the various DFFs. In the case of cell-free assays comprising membrane-bound forms, a solubilizing agent can be used to maintain DFF in solution. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON® X-100 and others from the TRITON® series, THESIT®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

(d) Immobilization of Target Molecules to Facilitate Screening

In more than one embodiment of the assay methods, immobilizing either a DFF or one of its partner molecules can facilitate separation of complexed from uncomplexed forms of one or both of the polypeptides, as well as to accommodate high throughput assays. Binding of a test compound to a DFF, or interaction of a DFF with a target molecule in the presence and absence of a candidate compound, can be accomplished in any vessel suitable for containing the reactants, such as microtiter plates, test tubes, and micro-centrifuge tubes. A fusion polypeptide can be provided that adds a domain that allows one or both of the polypeptides to be bound to a matrix. For example, GST-DFF fusion polypeptides or GST-target fusion polypeptides can be adsorbed onto glutathione sepharose beads (SIGMA Chemical, St. Louis, Mo.) or glutathione derivatized microtiter plates that are then combined with the test compound or the test compound and either the non-adsorbed target polypeptide or a DFF, and the mixture is incubated under conditions conducive to complex formation (e.g., at physiological conditions for salt and pH). Following incubation, the beads or microtiter plate wells are washed to remove any unbound components, the matrix immobilized in the case of beads, and complex formation determined either directly or indirectly. Alternatively, the complexes can be dissociated from the matrix, and the level of DFF binding or activity determined using standard techniques.

Other techniques for immobilizing polypeptides on matrices can also be used in screening assays. Either DFF or its target molecule can be immobilized using biotin-avidin or biotin-streptavidin systems. Biotinylation can be accomplished using many reagents, such as biotin-NHS (N-hydroxy-succinimide; Pierce Chemicals, Rockford, Ill.), and immobilized in wells of streptavidin-coated 96 well plates (Pierce Chemical). Alternatively, Abs reactive with a DFF or other target molecules, but which do not interfere with binding of a DFF to its target molecule, can be derivatized to the wells of the plate, and unbound target or DFF trapped in the wells by antibody conjugation. Methods for detecting such complexes, in addition to those described for the GST-immobilized complexes, include immunodetection of complexes using Abs reactive with DFF or its target, as well as enzyme-linked assays that rely on detecting an enzymatic activity associated with the DFF or target molecule.

(e) Screens to Identify Modulators

Modulators of the expression of a DFF can be identified in a method where a cell is contacted with a candidate compound and the expression of a DFF mRNA or polypeptide in the cell is determined. The expression level of a DFF mRNA or polypeptide in the presence of the candidate compound is compared to DFF mRNA or polypeptide levels in the absence of the candidate compound. The candidate compound can then be identified as a modulator of a DFF mRNA or polypeptide expression based upon this comparison. For example, when expression of a DFF mRNA or polypeptide is greater (i.e., statistically significant) in the presence of the candidate compound than in its absence, the candidate compound is identified as a stimulator of that DFF mRNA or polypeptide expression. Alternatively, when expression of a DFF mRNA or polypeptide is less (statistically significant) in the presence of the candidate compound than in its absence, the candidate compound is identified as an inhibitor of that DFF mRNA or polypeptide expression. The level of DFF mRNA or polypeptide expression in cells can be determined by methods described for detecting DFF mRNA or polypeptide.

(i) Hybrid Assays

In yet another aspect of the invention, DFFs can be used as “bait” in two- or three-hybrid assays (Bartel et al., 1993; Brent et al., WO94/10300, 1994; Iwabuchi et al., 1993; Madura et al., 1993; Saifer et al., U.S. Pat. No. 5,283,317, 1994; Zervos et al., 1993) to identify other polypeptides that bind or interact with DFFs and modulate DFF activities. Such DFF-binding partners are also likely to be involved in the propagation of signals by the DFFs as, for example, upstream or downstream elements of a DFF pathway.

The two-hybrid system is based on the modular nature of most transcription factors, which consist of separable DNA-binding and activation domains. Briefly, the assay utilizes two different DNA constructs. In one construct, the gene that codes for a DFF is fused to a gene encoding the DNA binding domain of a known transcription factor (e.g., GAL4). The other construct, a DNA sequence from a library of DNA sequences that encodes an unidentified polypeptide (“prey” or “sample”) is fused to a gene that codes for the activation domain of the known transcription factor. If the “bait” and the “prey” polypeptides are able to interact in vivo, forming a DFF-dependent complex, the DNA-binding and activation domains of the transcription factor are brought into close proximity. This proximity allows transcription of a reporter gene (e.g., LacZ) that is operably-linked to a transcriptional regulatory site responsive to the transcription factor. Expression of the reporter gene can be detected, and cell colonies containing the functional transcription factor can be isolated and used to obtain the cloned gene that encodes the DFF-interacting polypeptide.

The invention further pertains to novel agents identified by the aforementioned screening assays and their uses for treatments as described herein.

Detection Assays

Portions or fragments of DFF cDNA sequences—and the complete DFF gene sequences—are useful in themselves. These sequences can be used to: (1) identify an individual from a minute biological sample (tissue typing); and (2) aid in forensic identification of a biological sample.

The DFF sequences of the invention can be used to identify individuals from minute biological samples. In this technique, an individual's genomic DNA is digested with one or more restriction enzymes and probed on a Southern blot to yield unique bands. The sequences of the invention are useful as additional DNA markers for “restriction fragment length polymorphisms” (RFLP; (Smulson et al., U.S. Pat. No. 5,272,057, 1993)).

Furthermore, DFF sequences can be used to determine the actual base-by-base DNA sequence of targeted portions of an individual's genome. DFF sequences can be used to prepare two PCR primers from the 5′- and 3′-termini of the sequences that can then be used to amplify an the corresponding sequences from an individual's genome and then sequence the amplified fragment.

Panels of corresponding DNA sequences from individuals can provide unique individual identifications, as each individual will have a unique set of such DNA sequences due to allelic differences. The sequences of the invention can be used to identify such sequences from individuals and from tissue. The DFF sequences of the invention uniquely represent portions of an individual's genome. Allelic variation occurs to some degree in the coding regions of these sequences, and to a greater degree in the noncoding regions. The allelic variation between individual humans occurs with a frequency of about once ever 500 bases. Much of the allelic variation is due to single nucleotide polymorphisms (SNPs), which include RFLPs.

Each DFF sequences can, to some degree, be used as standards against which DNA from an individual can be compared for identification purposes. Because greater numbers of polymorphisms occur in noncoding regions, fewer sequences are necessary to differentiate individuals. Noncoding sequences can positively identify individuals with a panel of 10 to 1,000 primers that each yield a noncoding amplified sequence of 100 bases. If predicted coding sequences, such as those in SEQ ID NOS: 1, 6 or 11 are used, a more appropriate number of primers for positive individual identification would be 500-2,000.

Predictive Medicine

The invention also pertains to the field of predictive medicine in which diagnostic assays, prognostic assays, pharmacogenomics, and clinical trial monitoring are used for prognostic (predictive) purposes to treat an individual prophylactically. Accordingly, one aspect of the invention relates to diagnostic assays for determining DFF and/or nucleic acid expression as well as DFF activity, in the context of a biological sample (e.g., blood, serum, cells, tissue) to determine whether an individual is afflicted with a disease or disorder, or is at risk of developing a disorder, associated with aberrant DFF expression or activity, including cancer. The invention also provides for prognostic (or predictive) assays for determining whether an individual is at risk of developing a disorder associated with a DFF nucleic acid expression or activity. For example, mutations in a DFF can be assayed in a biological sample. Such assays can be used for prognostic or predictive purpose to prophylactically treat an individual prior to the onset of a disorder characterized by or associated with DFF, nucleic acid expression, or biological activity.

Another aspect of the invention provides methods for determining a DFF activity or nucleic acid expression in an individual to select appropriate therapeutic or prophylactic agents for that individual (pharmacogenomics). Pharmacogenomics allows for the selection of modalities (e.g., drugs, foods) for therapeutic or prophylactic treatment of an individual based on the individual's genotype (e.g., the individual's genotype to determine the individual's ability to respond to a particular agent). Another aspect of the invention pertains to monitoring the influence of modalities (e.g., drugs, foods) on the expression or activity of a DFF in clinical trials.

Diagnostic Assays

An exemplary method for detecting the presence or absence of DFF in a biological sample involves obtaining a biological sample from a subject and contacting the biological sample with a compound or an agent capable of detecting a DFF or a DFF nucleic acid such that the presence of DFF is confirmed in the sample. An agent for detecting a DFF message or DNA is a labeled nucleic acid probe that specifically hybridizes the target DFF mRNA or genomic DNA. The nucleic acid probe can be, for example, a full-length DFF nucleic acid, such as the nucleic acid of SEQ ID NOS: 1, 6 or 11 or a portion thereof, such as an oligonucleotide of at least 15, 30, 50, 100, 250 or 500 nucleotides in length and sufficient to specifically hybridize under stringent conditions to DFF mRNA or genomic DNA.

An agent for detecting a DFF polypeptide is an antibody capable of binding to DFF, preferably an antibody with a detectable label. Abs can be polyclonal, or more preferably, monoclonal. An intact antibody, or a fragment (e.g., F_{ab} or F(ab′)₂) can be used. A labeled probe or antibody is coupled (i.e., physically linking) to a detectable substance, as well as indirect detection of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently-labeled streptavidin. The term “biological sample” includes tissues, cells and biological fluids isolated from a subject, as well as tissues, cells and fluids present within a subject. Biological samples from a subject contains polypeptide molecules, and/or mRNA molecules, and/or genomic DNA molecules. A preferred biological sample is blood. Detection methods can be used to detect a DFF mRNA, polypeptide, or genomic DNA in a biological sample in vitro as well as in vivo. For example, in vitro techniques for detection of a DFF mRNA include Northern and in situ hybridizations. In vitro techniques for detection of a DFF polypeptide include enzyme-linked immunosorbent assays (ELISAs), Western blots, immunoprecipitations and immunofluorescence. In vitro techniques for detection of a DFF genomic DNA include Southern hybridizations and fluorescent in situ hybridization (FISH). Furthermore, in vivo techniques for detecting a DFF include introducing into a subject a labeled anti-DFF antibody. For example, the antibody can be labeled with a radioactive marker whose presence and location in a subject can be detected by standard imaging techniques.

The methods further involve obtaining a biological sample from a subject to provide a control, contacting the sample with a compound or agent to detect a DFF, and comparing the presence of DF in the control sample with the presence of DFF, mRNA or genomic DNA in the test sample.

Kits for detecting DFF in a biological sample may comprise a labeled compound or agent capable of detecting a DFF mRNA or polypeptide in a sample; reagent(s) and/or equipment for determining the amount of a DFF in the sample; and reagent(s) and/or equipment for comparing the amount of a DFF in the sample with a standard.

Prognostic Assays

Diagnostic methods can furthermore be used to identify subjects having, or at risk of developing, a disease or disorder associated with aberrant DFF expression or activity, such as obesity or obesity-related complications. Prognostic assays can be used to identify a subject having or at risk for developing a disease or disorder. A method for identifying a disease or disorder associated with aberrant DFF expression or activity would include a test sample obtained from a subject and detecting a DFF or nucleic acid (e.g., mRNA, genomic DNA). A test sample is a biological sample obtained from a subject. For example, a test sample can be a biological fluid (e.g., serum), cell sample, or tissue.

Prognostic assays can be used to determine whether a subject can be administered a modality (e.g., an agonist, antagonist, peptidomimetic, polypeptide, peptide, nucleic acid, small molecule, food, etc.) to treat a disease or disorder associated with aberrant DFF expression or activity. Such methods can be used to determine whether a subject can be effectively treated with an agent for a disorder, such as obesity. Methods for determining whether a subject can be effectively treated with an agent include obtaining a test sample and detecting a DFF or nucleic acid (e.g., where the presence of the DFF or nucleic acid is diagnostic for a subject that can be administered the agent to treat a disorder associated with aberrant DFF expression or activity).

Genetic lesions in a DFF can be used to determine if a subject is at risk for a disorder, such as obesity. Methods include detecting, in a sample from the subject, the presence or absence of a genetic lesion characterized by at an alteration affecting the integrity of a gene encoding a DFF polypeptide or the mis-expression of DFF. Such genetic lesions can be detected by ascertaining: (1) a deletion of one or more nucleotides from DFF; (2) an addition of one or more nucleotides to DFF; (3) a substitution of one or more nucleotides in DFF, (4) a chromosomal rearrangement of a DFF gene; (5) an alteration in the level of a DFF mRNA transcripts, (6) aberrant modification of a DFF, such as a change genomic DNA methylation, (7) the presence of a non-wild-type splicing pattern of a DFF mRNA transcript, (8) a non-wild-type level of DFF, (9) allelic loss of DFF, and/or (10) inappropriate post-translational modification of DFF polypeptide. There are a large number of known assay techniques that can be used to detect lesions in DFF. Any biological sample containing nucleated cells may be used.

In certain embodiments, lesion detection may use a probe/primer in a polymerase chain reaction (PCR) (e.g., (Mullis, U.S. Pat. No. 4,683,202, 1987; Mullis et al., U.S. Pat. No. 4,683,195, 1987), such as anchor PCR or rapid amplification of cDNA ends (RACE) PCR, or, alternatively, in a ligation chain reaction (LCR) (e.g., (Landegren et al., 1988; Nakazawa et al., 1994), the latter is particularly useful for detecting point mutations in DFF-genes (Abravaya et al., 1995). This method includes collecting a sample from a patient, isolating nucleic acids from the sample (if necessary), contacting the nucleic acids with one or more primers that specifically hybridize to DFF under conditions such that hybridization and amplification of the DFF (if present) occurs, and detecting the presence or absence of an amplification product, or detecting the size of the amplification product and comparing the length to a control sample. PCR and/or LCR are often desirable to use as a preliminary amplification step in conjunction with any of the techniques used for detecting mutations.

Alternative amplification methods include self sustained sequence replication (Guatelli et al., 1990), transcriptional amplification system (Kwoh et al., 1989); Qβ Replicase (Lizardi et al., 1988), or any other nucleic acid amplification method, followed by the detection of the amplified molecules using techniques well known to those of skill in the art. These detection schemes are especially useful for the detection of low abundant nucleic acid molecules.

Mutations in DFF from a sample can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes can be used to score for the presence of specific mutations by development or loss of a ribozyme cleavage site.

Hybridizing a sample and control nucleic acids, e.g., DNA or RNA, to high-density arrays containing hundreds or thousands of oligonucleotides probes, can identify genetic mutations in DFF (Cronin et al., 1996; Kozal et al., 1996). For example, genetic mutations in DFF can be identified in two-dimensional arrays containing light-generated DNA probes (Cronin et al., 1996). Briefly, a first hybridization array of probes can be used to scan through long stretches of DNA in a sample and control to identify base changes between the sequences by making linear arrays of sequential overlapping probes. This step allows the identification of point mutations. A second hybridization array follows that allows the characterization of specific mutations by using smaller, specialized probe arrays complementary to all variants or mutations detected. Each mutation array is composed of parallel probe sets, one complementary to the wild-type gene and the other complementary to the mutant gene.

Any of a variety of sequencing reactions known in the art can be used to directly sequence the target DFF and detect mutations by comparing the sequence of the sample DFF-with the corresponding wild-type (control) sequence. Examples of sequencing reactions include those based on classic techniques (Maxam and Gilbert, 1977; Sanger et al., 1977). Any of a variety of automated sequencing procedures can be used when performing diagnostic assays (Naeve et al., 1995) including sequencing by mass spectrometry (Cohen et al., 1996; Griffin and Griffin, 1993; Koster, WO94/16101, 1994).

Other methods for detecting mutations in the DFF include those in which protection from cleavage agents is used to detect mismatched bases in RNA/RNA or RNA/DNA heteroduplexes (Myers et al., 1985). In general, the technique of “mismatch cleavage” starts by providing heteroduplexes formed by hybridizing (labeled) RNA or DNA containing the wild-type DFF sequence with potentially mutant RNA or DNA obtained from a sample. The double-stranded duplexes are treated with an agent that cleaves single-stranded regions of the duplex such as those that arise from base pair mismatches between the control and sample strands. For instance, RNA/DNA duplexes can be treated with RNase and DNA/DNA hybrids treated with S₁ nuclease to enzymatically digest the mismatched regions. In other embodiments, either DNA/DNA or RNA/DNA duplexes can be treated with hydroxylamine or osmium tetroxide and with piperidine in order to digest mismatched regions. The digested material is then separated by size on denaturing polyacrylamide gels to determine the mutation site (Grompe et al., 1989; Saleeba and Cotton, 1993). The control DNA or RNA can be labeled for detection.

Mismatch cleavage reactions may employ one or more polypeptides that recognize mismatched base pairs in double-stranded DNA (DNA mismatch repair) in defined systems for detecting and mapping point mutations in DFF cDNAs obtained from samples of cells. For example, the mutY enzyme of E. coli cleaves A at G/A mismatches and the thymidine DNA glycosylase from HeLa cells cleaves T at G/T mismatches (Hsu et al., 1994). According to an exemplary embodiment, a probe based on a wild-type DFF sequence is hybridized to a cDNA or other DNA product from a test cell(s). The duplex is treated with a DNA mismatch repair enzyme, and the cleavage products, if any, can be detected from electrophoresis protocols or the like (Modrich et al., U.S. Pat. No. 5,459,039, 1995).

Electrophoretic mobility alterations can be used to identify mutations in DFF. For example, single strand conformation polymorphism (SSCP) may be used to detect differences in electrophoretic mobility between mutant and wild type nucleic acids (Cotton, 1993; Hayashi, 1992; Orita et al., 1989). Single-stranded DNA fragments of sample and control DFF nucleic acids are denatured and then renatured. The secondary structure of single-stranded nucleic acids varies according to sequence; the resulting alteration in electrophoretic mobility allows detection of even a single base change. The DNA fragments may be labeled or detected with labeled probes. Assay sensitivity can be enhanced by using RNA (rather than DNA), in which the secondary structure is more sensitive to a sequence changes. The method may use heteroduplex analysis to separate double stranded heteroduplex molecules on the basis of changes in electrophoretic mobility (Keen et al., 1991).

The migration of mutant or wild-type fragments can be assayed using denaturing gradient gel electrophoresis (DGGE; (Myers et al., 1985). In DGGE, DNA is modified to prevent complete denaturation, for example by adding a GC clamp of approximately 40 bp of high-melting, GC-rich DNA by PCR. A temperature gradient may also be used in place of a denaturing gradient to identify differences in the mobility of control and sample DNA (Rossiter and Caskey, 1990).

Examples of other techniques for detecting point mutations include selective oligonucleotide hybridization, selective amplification, or selective primer extension. For example, oligonucleotide primers can be prepared in which the known mutation is placed centrally and then hybridized to target DNA under conditions that permit hybridization only if a perfect match is found (Saiki et al., 1986; Saiki et al., 1989). Such allele-specific oligonucleotides are hybridized to PCR-amplified target DNA or a number of different mutations when the oligonucleotides are attached to the hybridizing membrane and hybridized with labeled target DNA.

Alternatively, allele specific amplification technology that depends on selective PCR amplification may be used. Oligonucleotide primers for specific amplifications may carry the mutation of interest in the center of the molecule (so that amplification depends on differential hybridization (Gibbs et al., 1989)) or at the extreme 3′-terminus of one primer where, under appropriate conditions, mismatch can prevent, or reduce polymerase extension (Prosser, 1993). Novel restriction sites in the region of the mutation may be introduced to create cleavage-based detection (Gasparini et al., 1992). Amplification may also be performed using Taq ligase (Barany, 1991). In such cases, ligation occurs only if there is a perfect match at the 3′-terminus of the 5′ sequence, allowing detection of a known mutation by scoring for amplification.

The described methods may be performed, for example, by using pre-packaged kits comprising at least one probe (nucleic acid or antibody) that may be conveniently used in clinical settings to diagnose patients exhibiting symptoms or family history of a disease or illness involving a DFF.

Furthermore, any cell type or tissue in which a DFF is expressed may be utilized in prognostic assays.

Pharmacogenomics

Agents, or modulators that have a stimulatory or inhibitory effect on a DFF activity or expression, as identified by a screening assay, can be administered to individuals to treat prophylactically or therapeutically disorders. In conjunction with such treatment, the pharmacogenomics (i.e., the study of the relationship between a subject's genotype and the subject's response to a foreign modality, such as a food, compound or drug) may be considered. Metabolic differences of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Pharmacogenomics can further be used to determine appropriate dosages and therapeutic regimens. Accordingly, the activity of a DFF, expression of a DFF nucleic acid, or DFF mutation(s) in an individual can be determined to guide the selection of appropriate agent(s) for therapeutic or prophylactic treatment.

Pharmacogenomics deals with clinically significant hereditary variations in the response to modalities due to altered modality disposition and abnormal action in affected persons (Eichelbaum and Evert, 1996; Linder et al., 1997). In general, two pharmacogenetic conditions can be differentiated: (1) genetic conditions transmitted as a single factor altering the interaction of a modality with the body (altered drug action) or (2) genetic conditions transmitted as single factors altering the way the body acts on a modality (altered drug metabolism). These pharmacogenetic conditions can occur either as rare defects or as nucleic acid polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) explains the phenomena of some patients who show exaggerated drug response and/or serious toxicity after taking the standard and safe dose of a drug. These polymorphisms are expressed in two phenotypes in the population, the extensive metabolizer (EM) and poor metabolizer (PM). The prevalence of PM is different among different populations. For example, the CYP2D6 gene is highly polymorphic and several mutations have been identified in PM, which all lead to the absence of functional CYP2D6. Poor metabolizers due to mutant CYP2D6 and CYP2C19 frequently experience exaggerated drug responses and side effects when they receive standard doses. If a metabolite is the active therapeutic moiety, PM shows no therapeutic response, as demonstrated for the analgesic effect of codeine mediated by its CYP2D6-formed metabolite morphine. At the other extreme are the so-called ultra-rapid metabolizers who are unresponsive to standard doses. Recently, the molecular basis of ultra-rapid metabolism has been identified to be due to CYP2D6 gene amplification.

The activity of a DFF, expression of a DFF nucleic acid, or mutation content of a DFF in an individual can be determined to select appropriate agent(s) for therapeutic or prophylactic treatment of the individual. In addition, pharmacogenetic studies can be applied to genotyping polymorphic alleles encoding drug-metabolizing enzymes to identify an individual's drug responsiveness phenotype. This knowledge, when applied to dosing or drug selection, can avoid adverse reactions or therapeutic failure and thus enhance therapeutic or prophylactic efficiency when treating a subject with a DFF modulator, such as a modulator identified by one of the described exemplary screening assays.

Monitoring Effects During Clinical Trials

Monitoring the influence of agents (e.g., drugs, compounds) on the expression or activity of a DFF can be applied not only in basic drug screening, but also in clinical trials. For example, the effectiveness of an agent determined by a screening assay to increase expression of a DFF, polypeptide levels, or up-regulate a DFF activity can be monitored in clinical trails of subjects exhibiting decreased DFF expression, polypeptide levels, or down regulated DFF activity. Alternatively, the effectiveness of an agent determined to decrease DFF expression, polypeptide levels, or down-regulate a DFF activity, can be monitored in clinical trials of subjects exhibiting increased DFF expression, polypeptide levels, or up regulated DFF activity. In such clinical trials, the expression or activity of a DFF and, preferably, other genes that have been implicated in, for example, obesity, can be used as a “read out” or markers for a particular cell's responsiveness.

For example, genes, including DFF, that are modulated in cells by treatment with a modality (e.g., food, compound, drug or small molecule) can be identified. To study the effect of agents on obesity, for example, in a clinical trial, cells can be isolated and RNA prepared and analyzed for the levels of expression of a DFF and other genes implicated in obesity. The gene expression pattern can be quantified by Northern blot analysis, nuclear run-on or RT-PCR experiments, or by measuring the amount of polypeptide, or by measuring the activity level of a DFF or other gene products. In this manner, the gene expression pattern itself can serve as a marker, indicative of the cellular physiological response to the agent. Accordingly, this response state may be determined before, and at various points during, treatment of the individual with the agent.

The invention provides a method for monitoring the effectiveness of treatment of a subject with an agent (e.g., an agonist, antagonist, polypeptide, peptide, peptidomimetic, nucleic acid, small molecule, food or other drug candidate identified by the screening assays described herein) comprising the steps of (1) obtaining a pre-administration sample from a subject; (2) detecting the level of expression of a DFF, mRNA, or genomic DNA in the preadministration sample; (3) obtaining one or more post-administration samples from the subject; (4) detecting the level of expression or activity of the DFF, mRNA, or genomic DNA in the post-administration samples; (5) comparing the level of expression or activity of the DFF, mRNA, or genomic DNA in the pre-administration sample with the DFF, mRNA, or genomic DNA in the post administration sample or samples; and (6) altering the administration of the agent to the subject accordingly. For example, increased administration of the agent may be desirable to increase the expression or activity of DFF to higher levels than detected, i.e., to increase the effectiveness of the agent. Alternatively, decreased administration of the agent may be desirable to decrease expression or activity of DFF to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant DFF expression or activity, such as obesity.

Disease and Disorders

Diseases and disorders that are characterized by increased DFF levels or biological activity may be treated with therapeutics that antagonize (i.e., reduce or inhibit) activity. Antagonists may be administered in a therapeutic or prophylactic manner. Therapeutics that may be used include: (1) DFF peptides, or analogs, derivatives, fragments or homologs thereof; (2) Abs to a DFF peptide; (3) DFF nucleic acids; (4) administration of antisense nucleic acid and nucleic acids that are “dysfunctional” (i.e., due to a heterologous insertion within the coding sequences) that are used to eliminate endogenous function of by homologous recombination (Capecchi, 1989); or (5) modulators (i.e., inhibitors, agonists and antagonists, including additional peptide mimetic of the invention or Abs specific to DFF) that alter the interaction between DFF and its binding partner.

Diseases and disorders that are characterized by decreased DFF levels or biological activity may be treated with therapeutics that increase (i.e., are agonists to) activity. Therapeutics that upregulate activity may be administered therapeutically or prophylactically. Therapeutics that may be used include peptides, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying in vitro for RNA or peptide levels, structure and/or activity of the expressed peptides (or DFF mRNAs). Methods include immunoassays (e.g., Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

The invention provides a method for preventing, in a subject, a disease or condition associated with an aberrant DFF expression or activity, by administering an agent that modulates expression of a DFF or at least one DFF activity. Subjects at risk for a disease that is caused or contributed to by aberrant DFF expression or activity can be identified by, for example, any or a combination of diagnostic or prognostic assays. Administration of a prophylactic agent can occur prior to the manifestation of symptoms characteristic of the DFF aberrancy, such that a disease or disorder is prevented or, alternatively, delayed in its progression. Depending on the type of DFF aberrancy, for example, a DFF agonist or antagonist can be used to treat the subject. The appropriate agent can be determined based on screening assays.

Therapeutic Methods

Modulating DFF expression or activity can be used therapeutically. Such a modulatory method involves contacting a cell with an agent that modulates one or more of the activities of a DFF activity associated with the cell. An agent that modulates a DFF activity can be a nucleic acid or a polypeptide, a naturally occurring cognate ligand of a DFF, a peptide, a DFF peptidomimetic, or other small molecule. The agent may stimulate a DFF activity. Examples of such stimulatory agents include active DFF, and a DFF nucleic acid molecule that has been introduced into the cell. In another embodiment, the agent inhibits a DFF activity. Examples of inhibitory agents include antisense DFF and anti-DFF Abs. Modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a DFF polypeptide or nucleic acid. For example, the method involves administering an agent (e.g., an agent identified by a screening assay), or combination of agents that modulates (e.g., up-regulates or down-regulates) DFF expression or an activity. Alternatively, the method involves administering a DFF or nucleic acid molecule as therapy to compensate for reduced or aberrant DFF expression or activity.

Stimulation of a DFF activity is desirable in situations in which a DFF is abnormally down regulated and/or in which increased DFF activity is likely to have a beneficial effect.

Determination of the Biological Effect of the Therapeutic

Suitable in vitro or in vivo assays can be performed to determine the effect of a specific therapeutic and whether its administration is indicated for treatment of the affected tissue.

In various specific embodiments, in vitro assays may be performed with representative cells of the type(s) involved in the patient's disorder, to determine if a given therapeutic exerts the desired effect upon the cell type(s). Modalities for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

DFF nucleic acids and polypeptides are useful in potential prophylactic and therapeutic applications implicated in a variety of disorders including obesity.

As an example, a cDNA encoding a DFF may be useful in gene therapy, and the polypeptide may be useful when administered to a subject in need. The compositions of the invention will have efficacy for treatment of patients suffering from obesity.

DFF nucleic acids or fragments are also useful in diagnostic applications, wherein the presence or amount of the nucleic acid or the polypeptide is to be assessed. A further use could be as an anti-bacterial molecule (i.e., some peptides have been found to possess anti-bacterial properties). These materials are further useful in the generation of Abs that immunospecifically bind to the novel substances of the invention for use in therapeutic or diagnostic methods.

### EXAMPLES

The following examples are included to demonstrate preferred embodiments of the present invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1 Fasting/Feeding Experiments

Experimental Design Details

:Five groups of mice. n=5/group.

1. Ad lib fed mice.

2. Mice fasted for 4 hours.

3. Mice fasted for 24 hours.

4. Mice fasted for 48 hours

5. Mice fasted for 48 hours and then refed for 24 hours.

All studies were done in accordance with guidelines set forth by the Institutional Animal Care and Use Committee at Genentech. Male FVB-N/J mice (Jackson Labs, Bar Harbor, Me., USA) were received at 3 wk of age and housed at 2 mice/cage until tissue harvest at 6 wk of age. All mice were fed rodent chow ad libitum (Chow 5010, Ralston Purina; St. Louis, Mo., USA) and housed on a 12:12 light/dark cycle (lights on 06:00) at 22° C. Following CO₂-induced euthanasia, stomach tissue was excised, carefully cleaned, and snap-frozen in liquid nitrogen for subsequent RNA preparation.

Samples from each treatment group were transferred to CuraGen Corp. (New Haven, Conn., USA), RNA prepared and reverse-transcribed, and subjected to Quantitative Expression Analysis (QEA) (Shimkets, et al., 1999).

### Example 2 GeneCalling (Shimkets et al., 1999)

RNA Isolation

Total RNA was isolated with Trizol (Life Technologies, Grand Island, N.Y.) using 0.1 volume of bromochloropropane for phase separation (Molecular Research Center, Cincinnati, Ohio), and treated with DNase I (Promega, Madison, Wis.) in the presence of 0.01 M dithiothreitol (DTT) and 1 U/1 RNasin (Promega). Following phenol/chloroform extraction, RNA quality was evaluated by spectrophotometry and formaldehyde agarose gel electrophoresis, and yield was estimated by fluorometry with OliGreen (Molecular Probes, Eugene, Oreg.). Poly-A+RNA was prepared from 100 g total RNA using oligo(dT) magnetic beads (PerSeptive, Cambridge, Mass.), and quantified with fluorometry.

First-strand cDNA was prepared from 1.0 g of poly(A)+RNA with 200 pmol oligo(dT)25V (V=A, C or G) using 400 U of Superscript II reverse transcriptase (BRL). Second-strand synthesis was performed at 16° C. for 2 hours after addition of 10 U of E. coli DNA ligase, 40 U of E. coli DNA polymerase, and 3.5 U of E. coli RNase H (all from BRL). T4 DNA polymerase (5 U) was added, incubated for 5 min at 16° C., followed by treatment with arctic shrimp alkaline phosphatase (5 U; United States Biochemicals, Cleveland, Ohio) at 37° C. for 30 min. cDNA was purified by phenol/chloroform extraction, and the yield was estimated using fluorometry with PicoGreen (Molecular Probes).

cDNA fragmentation was achieved by digestion in a 50 μl reaction mixture containing 5 U of restriction enzyme (6 base-pair cutters) and 1 ng of double-stranded cDNA. Eighty separate sets of cDNA fragmentation reactions were conducted, each with a different pair of restriction enzymes. These were then ligated to complementary amplification tags with ends compatible to the 5′ and 3′ ends of the fragments at 16° C. for 1 hour in 10 mM ATP, 2.5% PEG, 10 units T4 DNA ligase, and 1 ligase buffer. Amplification was then performed after addition of 2 μl 10 mM dNTP, 5 μl 10 TB buffer (500 mM Tris, 160 mM (NH4)₂SO₄, 20 mM MgCl₂, pH 9.15), 0.25 μl Klentaq (Clontech Laboratories, Palo Alto, Calif.): PFU (Stratagene, La Jolla, Calif.) (16:1), 32.75 μl H2O. Amplification was carried out for 20 cycles (30 s at 96° C., 1 min at 57° C., 2 min at 72° C.), followed by 10 min at 72° C. PCR products were purified using streptavidin beads (CPG, Lincoln Park, N.J.). After washing the beads twice with buffer 1 (3 M NaCl, 10 mM Tris-HCl, 1 mM EDTA, pH 7.5), 20 μl of buffer 1 was mixed with the PCR product for 10 min at room temperature, separated with a magnet, and washed once with buffer 2 (10 mM Tris, 1 mM EDTA, pH 8.0). The beads were then dried and resuspended in 3 μl of buffer 3 (80% (vol/vol) formamide, 4 mM EDTA, 5% TAMRA- or ROX-tagged molecular size standards (PE-Applied Biosystems, Foster City, Calif.). Following denaturation (96° C. for 3 min), samples were loaded onto 5% polyacrylamide, 6 M urea, 0.5 Tris Borate EDTA ultrathin gels and electrophoresed on a Niagara instrument. PCR products were visualized by virtue of the fluorescent FAM label at the 5′ end of one of the PCR primers, which ensures that all detected fragments have been digested by both enzymes.

Gel Interpretation

Electrophoresis data was processed using the Open Genome Initiative (OGI) software. Gel images were first visually checked and tracked. Each lane contains the FAM-labeled products of a single reaction plus a sizing ladder spanning the range from 50 to 500 bp. The ladder peaks provide a correlation between camera frames (collected at 1 Hz) and DNA fragment size in base pairs. After tracking, lanes were extracted and the peaks in the sizing ladder were found. Linear interpolation between the ladder peaks converted the fluorescence traces from frames to base pairs. A final quality control step checked for low signal-to-noise, poor peak resolution, missing ladder peaks, and lane-to-lane bleed. Data that pass all of these criteria were submitted as point-by-point length versus amplitude addresses to an Oracle 8 database.

Difference Identification

For each restriction enzyme pair in each sample set a composite trace was calculated, compiling all the individual sample replicates followed by application of a scaling algorithm for best fit to normalize the traces of the experimental set versus that of the control. The scaled traces are then compared on a point-by-point basis to define areas of amplitude difference that meet the minimum prespecified threshold for a significant difference. Once a region of difference has been identified, the local maximum for the corresponding traces of each set was then determined. The variance of the difference was calculated by the following expression:

σ²Δ(j)=λ₁(j)²σ²_{Total} (j:S₁)+λ₂(j)²σ²_{Total}(j:S₂)

where λ₁(j) and λ₂(j) represent scaling factors and (j:S) represents the trace composite values over multiple samples. The probability that the difference is statistically significant is calculated by

$$P ( j ) = 1 - {\int }_{- Δ}^{Δ} ⅆ y ( 1 / √ \{ 2 π {σ}_{Δ}^{2} \} ) exp ( - {y}^{2} / 2 {σ}_{Δ}^{2} )$$

where y is the relative intensity. All difference peaks are stored as unique database addresses in the specified expression difference analysis.

Gene Confirmation by Oligonucleotide Poisoning

Restriction fragments that map in end sequence and length to known rat genes are used as templates for the design of unlabeled oligonucleotide primers. An unlabeled oligonucleotide designed against one end of the restriction fragment is added in excess to the original reaction, and is reamplified for an additional 15 cycles. This reaction is then electrophoresed and compared to a control reaction reamplified without the unlabeled oligonucleotide to evaluate the selective diminution of the peak of interest.

RNA Doping

DNA templates for RNA in vitro transcription were generated by PCR amplification using cloned human cDNAs as templates. PCR primers were complementary to plasmid sequences flanking the cDNA inserts. In addition, the sense primer contained the T7 RNA polymerase consensus sequence, and the antisense primer included a stretch of 25 thymidines for the generation of polyadenylated transcripts. In vitro transcription was performed using the MaxiScript transcription kit (Ambion, Austin, Tex.). The transcripts were poly-A selected on biotin-oligo(dT)25 bound to streptavidin MPG beads (CPG Inc.). The RNA products ranged in size between 1,100 and 2,000 nt. The integrity of the products was monitored by agarose gel electrophoresis, and the concentration determined by fluorometry using RiboGreen dye (Molecular Probes) on a SpectraFluor fluorometer (Tecan, Grundig, Austria). The in vitro transcribed RNAs were mixed at defined ratios with HeLa cell poly-A+RNA (American Type Culture Collection, Manassas, Va.) and the RNA was converted to cDNA and subjected to GeneCalling chemistry and analysis as described.

### Example 3 Identification of Glycerol Kinase

A novel mouse fragment was identified as differentially expressed, showing transient recovery after fasting following feeding. After cloning and poisoning, the mouse fragment was found by BlastX to be highly conserved in Pseudomonas as a glycerol kinase (GK). The Pseudomonas BlastX result was used to identify a region of human genomic DNA (gDNA; AC022123 sequence from Chromosome IV, consisting of 500 unordered pieces (SEQ ID NO: 17)) to look for the human homolog of the mouse fragment. GeneAngler with the mammalian GK identified a single exon. This exon was used in BlastP to find Homo sapiens GK testes-specific 2 which was then used with GeneAngler to test AC022123 (SEQ ID NO: 17) again. This resulted in the sequences SEQ ID NOs: 1 and 2 (glycerol kinase; GLK).

The GLK was poisoned originally as c-Krox. c-Krox appears to be actually upregulated, due to the “down” trace of the pass partial, showing that the relative expression of c-Krox is up in the experimental and down in the control. However, the down trace poisoning revealed that there was a differentially expressed band underneath the peak that was also down regulated and belonging to a different gene. This fragment did not have a good GeneCall, so it was isolated and poisoned to reveal the mouse fragment sequence:

| (SEQ ID NO:18) | |
|---|---|
| 1 | tgatcagcaa gcagcattgt tcggacaaat gtgcgtagaa gttggacaag ctaaaaacac |
| 61 | ttatggtacc |

This sequence is highly conserved among glycerol kinases from bacteria to humans.

The underlying peak was differentially expressed, too. Like the c-Krox, GLK is transiently up regulated after fasting induced down-regulation. After the refeeding after fasting, it is transiently up regulated (first 24 hours of refeeding) then it is down regulated between 24 and 48 hours of refeeding after fasting.

### Example 4 Identification of Putative Peroxisomal Membrane Associated Polypeptide (PMAP)

This sequence was assembled from the following components using CuraTools SeqExtender:

Consensus extension of cgmml0r0291.3_{—}38486-119 using the 127 sequences: cgmml0r0291.3_{—}38486-119−, est:gb_AA048825.1+, est:gb_AA049042.1+, est:gb_AA050066.1+, est:gb_AA087793.1−, est:gb_AA104115.1+, est:gb_AA108745.1+, est:gb_AA110375.1+, est:gb_AA110622.1+, est:gb_AA122560.1+, est:gb_AA140182.1+, est:gb_AA198115.1+, est:gb_AA412999.1+, est:gb_AA474253.1+, est:gb_AA589497.1+, est:gb_AA615178.1−, est:gb_AA645359.1+, est:gb_AA673449.1+, est:gb_AA688817.1+, est:gb _AA726176.1+, est:gb_AA734901.1+, est:gb_AA738662.1, est:gb_AA762739.1 +, est:gb_AA798938.1+, est:gb_AA930943.1+, est:gb_AI035922.1+, est:gb_AI037747.1+, est:gb_AI326111.1−, est:gb_AI386226.1+, est:gb_AI835182.1, est:gb_AI956516.1+, est:gb_AI956764.1, est:gb_AV002032.1+, est:gb_A V011160.1+, est:gb_AV052321.1+, est:gb_AV066912.1+, est:gb_AV086580.1+, est:gb_AV101531.1+, est:gb_AV116793.1+, est:gb_AV147721.1+, est:gb_AV149310.1+, est:gb_AV211982.1+, est:gb_AV213514.1+, est:gb_AV214067.1+, est:gb_AV217073.1+, est:gb_AV306358.1+, est:gb_AV322885.1+, est:gb_AV367037.1+, est:gb_AV369721.1+, est:gb_AV375317.1+, est:gb_AW012304.1+, est:gb_AW044982.1+, est:gb_AW047363.1−, est:gb_AW321786.1+, est:gb_AW476384.1+, est:gb_BB036278.1+, est:gb_BB057120.1+, est:gb_BB072183.1+, est:gb_BB088405.1+, est:gb_BB09025 1.1+, est:gb_BB094975.1+, est:gb_BB140129.1+, est:gb_BB160862.1+, est:gb_BB167124.1+, est:gb_BB188605.1+, est:gb_BB189580.1+, est:gb_BB206724.1+, est:gb_BB213590.1+, est:gb_BB213783.1+, est:gb_BB224477.1+, est:gb_BB254909.1+, est:gb_BB261723.1+, est:gb_BB291610.1+, est:gb_BB298572.1+, est:gb_BB316466.1+, est:gb_BB316696.1+, est:gb_BB345255.1+, est:gb_BB346818.1+, est:gb_BB400162.1+, est:gb_BB402315.1+, est:gb_BB409641.1+, est:gb_BB417420.1+, est:gb_BB499765.1+, est:gb_BB516623.1+, est:gb_BB520492.1+, est:gb_BB537841.1+, est:gb_BB559612.1+, est:gb_BB579257.1+, est:gb_BB585551.1+, est:gb_BB606066.1+, est:gb_BE134622.1+, est:gb_BE226279.1+, est:gb_BE284150.1+, est:gb_BE290265.1+, est:gb_BE376311.1+, est:gb_BE380398.1+, est:gb_BE623293.1, est:gb_BE647176.1+, est:gb_BE912918.1+, est:gb_BE982558.1−, est:gb_BE982885.1−, est:gb_BE984043.1−, est:gb_BE984102.1−, est:gb_BE989553.1−, est:gb_BE989696.1−, est:gb_BE990234.1−, est:gb_BE994465.1−, est:gb_BF137669.1+, est:gb_BF300298.1+, est:gb_BF456397.1−, est:gb_BF608791.1+, est:gb_BF780670.1+, est:gb_BG062889.1+, est:gb_BG175495.1+, est:gb_BG342253.1+, est:gb_BG518652.1+, est:gb_BG695107.1+, est:gb_BG866670.1+, est:gb_BG965006.1+, est:gb_BI101248.1+, est:gb_BI106954.1+, est:gb_W08093.1+, est:gb_W08700.1+, est:gb_W10271.1+, est:gb_W29897.1+, est:gb_W75821.1+, est:gb_W77401.1+

### Example 5 Identification of Trefoil Factor 1 (TFF1)/pS2

M.musculus mRNA for P domain polypeptide Z21858.1 (SEQ ID NO: 19) 100% identical. PS2 was induced during fasting, and down regulated with post-fasting feeding. This polypeptide has not previously been associated with metabolic phenomena, although does have a role in cancer cells.

References

U.S. Pat. No. 4,166,452. Apparatus for testing human responses to stimuli. 1979.

U.S. Pat. No. 4,485,045. Synthetic phosphatidyl cholines useful in forming liposomes. 1984.

U.S. Pat. No. 4,544,545. Liposomes containing modified cholesterol for organ targeting. 1985.

U.S. Pat. No. 4,676,980. Target specific cross-linked heteroantibodies. 1987.

U.S. Pat. No. 4,816,567. Recombinant immunoglobin preparations. 1989.

WO 90/10448. Covalent conjugates of lipid and oligonucleotide. 1990.

WO 90/13641. Stably transformed eucaryotic cells comprisng a foreign transcribable DNA under the control of a pol III promoter. 1990.

EPO 402226. Transformation vectors for yeast Yarrowia. 1990.

WO 91/00360. Bispecific reagents for AIDS therapy. 1991.

WO 91/04753. Conjugates of antisense oligonucleotides and therapeutic uses thereof. 1991.

U.S. Pat. No. 5,013,556. Liposomes with enhanced circulation time. 1991.

WO 91/06629. Oligonucleotide analogs with novel linkages. 1991.

WO 92/20373. Heteroconjugate antibodies for treatment of HIV infection. 1992.

WO 93/08829. Compositions that mediate killing of HIV-infected cells. 1993.

WO 94/11026. Therapeutic application of chimeric and radiolabeled antibodies to human B lymphocyte restricted differentiation antigen for treatment of B cells. 1994.

WO 96/27011. A method for making heteromultimeric polypeptides. 1996.

U.S. Pat. No. 5,545,807. Production of antibodies from transgenic animals. 1996.

U.S. Pat. No. 5,569,825. Transgenic non-human animals capable of producing heterologous antibodies of various isotypes. 1996.

WO 97/33551. Compositions and methods for the diagnosis, prevention, and treatment of neoplastic cell growth and proliferation. 1997.

U.S. Pat. No. 5,633,425. Transgenic non-human animals capable of producing heterologous antibodies. 1997.

U.S. Pat. No. 5,661,016. Transgenic non-human animals capable of producing heterologous antibodies of various isotypes. 1997.

U.S. Pat. No. 5,625,126. Transgenic non-human animals for producing heterologous antibodies. 1997.

Abravaya, K., J. J. Carrino, S. Muldoon, and H. H. Lee. 1995. Detection of point mutations with a modified ligase chain reaction (Gap- LCR). Nucleic Acids Res. 23:675-82.

Alam, J., and J. L. Cook. 1990. Reporter genes: Application to the study of mammalian gene transcription. Anal. Biochem. 188:245-254.

Altschul, S. F., W. Gish, W. Miller, E. W. Myers, et al. 1990. Basic local alignment search tool. J Mol Biol. 215:403-10.

Altschul, S. F., T. L. Madden, A. A. Schaffer, J. Zhang, et al. 1997. Gapped BLAST and PSI-BLAST: a new generation of protein database search programs. Nucleic Acids Res. 25:3389-402.

Aron, D., J. Findling, and J. Tyrrell. 1997. Hypothalamus and pituitary. In Basic & clinical endocrinology. F. Greenspan and G. Strewler, editors. Appleton & Lange, Stamford. 95-156.

Austin, C. P., and C. L. Cepko. 1990. Cellular migration patterns in the developing mouse cerebral cortex. Development. 110:713-732.

Ausubel, F. M., R. Brent, R. E. Kingston, D. D. Moore, et al. 1987. Current protocols in molecular biology. John Wiley & Sons, New York.

Barany, F. 1991. Genetic disease detection and DNA amplification using cloned thermostable ligase. Proc Natl Acad Sci USA. 88:189-93.

Bartel, D. P., and J. W. Szostak. 1993. Isolation of new ribozymes from a large pool of random sequences [see comment]. Science. 261:1411-8.

Bartel, P., C. T. Chien, R. Stemglanz, and S. Fields. 1993. Eliminaton of false positives that arise in using the two-hybrid system. Biotechniques. 14:920-4.

Beal, P. A., and P. B. Dervan. 1991. Second structural motif for recognition of DNA by oligonucleotide-directed triple-helix formation. Science. 251:1360-3.

Bechtold, N., and G. Pelletier. 1998. In planta Agrobacterium-mediated transformation of adult Arabidopsis thaliana plants by vacuum infiltration. Methods Mol Biol. 82:259-66.

Beck, B. 2001. KO's and organisation of peptidergic feeding behavior mechanisms. Neurosci Biobehav Rev. 25:143-58.

Becker, D. M., and L. Guarente. 1991. High-efficiency transformation of yeast by electroporation. Methods Enzymol. 194:182-187.

Beggs, J. D. 1978. Transformation of yeast by a replicating hybrid plasmid. Nature. 275:104-109.

Berger, J., J. Hauber, R. Hauber, R. Geiger, et al. 1988. Secreted placental alkaline phosphatase: A powerful new qunatitative indicator of gene expression in eukaryotic cells. Gene. 66:1-10.

Berns, A., R. Mandag, and H. Te Riele. WO 93/04169. GENE TARGETING IN ANIMAL CELLS USING ISOGENIC DNA CONSTRUCTS. 1993.

Bodine, D. M., K. T. McDonagh, N. E. Seidel, and A. W. Nienhuis. 1991. Survival and retrovirus infection of murine hematopoietic stem cells in vitro: effects of 5-FU and method of infection. Exp. Hematol. 19:206-212.

Boerner, P., R. Lafond, W. Z. Lu, P. Brams, et al. 1991. Production of antigen-specific human monoclonal antibodies from in vitro-primed human splenocytes. J Immunol. 147:86-95.

Boswell, G. A., and R. M. Scribner. U.S. Pat. No. 3,773,919. Polylactide-drug mixtures. 1973.

Bradley. 1987. Teratocarcinomas and Embryonic Stem Cells: A Practical Approach. Oxford University Press, Inc., Oxford. 268 pp.

Bradley, A. 1991. Modifying the mammalian genome by gene targeting. Curr Opin Biotechnol. 2:823-9.

Brennan, M., P. F. Davison, and H. Paulus. 1985. Preparation of bispecific antibodies by chemical recombination of monoclonal immunoglobulin G1 fragments. Science. 229:81-3.

Brent, R., J. Gyuris, and E. Golemis. WO94/10300. INTERACTION TRAP SYSTEM FOR ISOLATING NOVEL PROTEINS. 1994.

Cancela, J. M. 2001. Specific Ca2+ signaling evoked by cholecystokinin and acetylcholine: the roles of NAADP, cADPR, and IP3. Annu Rev Physiol. 63:99-117.

Capecchi, M. R. 1980. High efficiency transformation by direct microinjection of DNA into cultured mammalian cells. Cell. 22:479.

Capecchi, M. R. 1989. Altering the genome by homologous recombination. Science. 244:1288-92.

Carell, T., E. A. Wintner, and J. Rebek Jr. 1994a. A novel procedure for the synthesis of libraries containing small organic molecules. Angewandte Chemie International Edition. 33:2059-2061.

Carell, T., E. A. Wintner, and J. Rebek Jr. 1994b. A solution phase screening procedure for the isolation of active compounds from a molecular library. Angewandte Chemie International Edition. 33:2061-2064.

Caron, P. C., W. Laird, M. S. Co, N. M. Avdalovic, et al. 1992. Engineered humanized dimeric forms of IgG are more effective antibodies. J Exp Med. 176:1191-5.

Carter, P. 1986. Site-directed mutagenesis. Biochem J. 237:1-7.

Case, M. E., M. Schweizer, S. R. Kushner, and N. H. Giles. 1979. Efficient transformation of Neurospora crassa by utilizing hybrid plasmid DNA. Proc Natl Acad Sci USA. 76:5259-63.

Cech, T. R., F. L. Murphy, and A. J. Zaug. U.S. Pat. No. 5,116,742. RNA ribozyme restriction endoribonucleases and methods. 1992.

Cech, T. R., A. J. Zaug, and M. D. Been. U.S. Pat. No. 4,987,071. RNA ribozyme polymerases, dephosphorylases, restriction endoribonucleases and methods. 1991.

Cepko, C. L., B. E. Roberts, and R. E. Mulligan. 1984. Construction and applications of a highly transmissible murine retrovirus shuttle vector. Cell. 37:1053-1062.

Chalfie, M., Y. tu, G. Euskirchen, W. W. Ward, et al. 1994. Green fluorescent protein as a marker for gene expression. Science. 263:802-805.

Chaney, W. G., D. R. Howard, J. W. Pollard, S. Sallustio, et al. 1986. High-frequency transfection of CHO cells using Polybrene. Somatic Cell Mol. Genet. 12:237.

Chen, C., and H. Okayama. 1988. Calcium phosphate-mediated gene transfer: A highly efficient system for stably transforming cells with plasmid DNA. BioTechniques. 6:632-638.

Chen, S. H., H. D. Shine, J. C. Goodman, R. G. Grossman, et al. 1994. Gene therapy for brain tumors: regression of experimental gliomas by adenovirus-mediated gene transfer in vivo. Proc Natl Acad Sci USA. 91:3054-7.

Cho, C. Y., E. J. Moran, S. R. Cherry, J. C. Stephans, et al. 1993. An unnatural biopolymer. Science. 261:1303-5.

Clement, K., C. Vaisse, N. Lahlou, S. Cabrol, et al. 1998. A mutation in the human leptin receptor gene causes obesity and pituitary dysfunction. Nature. 392:398-401.

Cohen, A. S., D. L. Smisek, and B. H. Wang. 1996. Emerging technologies for sequencing antisense oligonucleotides: capillary electrophoresis and mass spectrometry. Adv Chromatogr. 36:127-62.

Cohen, J. S. 1989. Oligodeoxynucleotides: Antisense inhibitors of gene expression. CRC Press, Boca Raton, Fla. 255 pp.

Cohen, S. M. N., A. C. Y. Chang, and L. Hsu. 1972. Nonchromosomal antibiotic resistance in bacteria: Genetic transformation of Escherichia coli by R-factor DNA. Proc. Natl. Acad. Sci. USA. 69:2110.

Comuzzie, A. G., and D. B. Allison. 1998. The search for human obesity genes. Science. 280:1374-7.

Cooney, M., G. Czernuszewicz, E. H. Postel, S. J. Flint, et al. 1988. Site-specific oligonucleotide binding represses transcription of the human c-myc gene in vitro. Science. 241:456-9.

Cotton, R. G. 1993. Current methods of mutation detection. Mutat Res. 285:125-44.

Cronin, M. T., R. V. Fucini, S. M. Kim, R. S. Masino, et al. 1996. Cystic fibrosis mutation detection by hybridization to light-generated DNA probe arrays. Hum Mutat. 7:244-55.

Cull, M. G., J. F. Miller, and P. J. Schatz. 1992. Screening for receptor ligands using large libraries of peptides linked to the C terminus of the lac repressor. Proc Natl Acad Sci USA. 89:1865-9.

Cwirla, S. E., E. A. Peters, R. W. Barrett, and W. J. Dower. 1990. Peptides on phage: a vast library of peptides for identifying ligands. Proc Natl Acad Sci USA. 87:6378-82.

de Boer, A. G. 1994. Drug absorption enhancement: Concepts, possibilities, limitations and trends. Harwood Academic Publishers, Langhorne, Pa.

de Louvencourt, L., H. Fukuhara, H. Heslot, and M. Wesolowski. 1983. Transformation of Kluyveromyces lactis by killer plasmid DNA. J Bacteriol. 154:737-42.

de Wet, J. R., K. V. Wood, M. DeLuca, D. R. Helinski, et al. 1987. Sturcture and expression in mammalian cells. Mol. Cell Biol. 7:725-737.

Demerec, M., E. A. Adelberg, A. J. Clark, and P. E. Hartman. 1966. A proposal for a uniform nomenclature in bacterial genetics. Genetics. 54:61-76.

Devlin, J. J., L. C. Panganiban, and P. E. Devlin. 1990. Random peptide libraries: a source of specific protein binding molecules. Science. 249:404-6.

DeWitt, S. H., J. S. Kiely, C. J. Stankovic, M. C. Schroeder, et al. 1993. “Diversomers”: an approach to nonpeptide, nonoligomeric chemical diversity. Proc Natl Acad Sci USA. 90:6909-13.

Ebihara, K., Y. Ogawa, H. Masuzaki, M. Shintani, et al. 2001. Transgenic overexpression of leptin rescues insulin resistance and diabetes in a mouse model of lipoatrophic diabetes. Diabetes. 50:1440-8.

Eichelbaum, M., and B. Evert. 1996. Influence of pharmacogenetics on drug disposition and response. Clin Exp Pharmacol Physiol. 23:983-5.

Ellington, A. D., and J. W. Szostak. 1990. In vitro selection of RNA molecules that bind specific ligands. Nature. 346:818-22.

Elroy-Stein, O., and B. Moss. 1990. Cytoplasmic expression system based on constitutive synthesis of bacteriophage T7 RNA polymerase in mammalian cells. Proc. Natl. Acad. Sci. USA. 87:6743-6747.

Eppstein, D. A., E. B. Fraser-Smith, and T. R. Mattews. U.S. Pat. No. 4,522,811. Serial injection of muramyldipeptides and liposomes enhances the anti-infective activity of muramyldipeptides Serial injection of muramyldipeptides and liposomes enhances the anti-infective activity of muramyldipeptides. 1985.

Eppstein, D. A., Y. V. Marsh, M. van der Pas, P. L. Felgner, et al. 1985. Biological activity of liposome-encapsulated murine interferon gamma is mediated by a cell membrane receptor. Proc Natl Acad Sci USA. 82:3688-92.

Escudero, J., and B. Hohn. 1997. Transfer and integration of T-DNA without cell injury in the host plant. Plant Cell. 9:2135-2142.

Evans, R., R. D. Palmiter, and R. L. Brinster. U.S. Pat. No. 4,870,009. Method of obtaining gene product through the generation of transgenic animals. 1989.

Farooqi, I. S., S. A. Jebb, G. Langmack, E. Lawrence, et al. 1999. Effects of recombinant leptin therapy in a child with congenital leptin deficiency. N Engl J Med. 341:879-84.

Fekete, D. M., and C. L. Cepko. 1993. Retroviral infection coupled with tissue transplantation limits gene transfer in the chick embryo. Proc. Natl. Acad. Sci. USA. 90:2350-2354.

Felgner, P. L., T. R. Gadek, M. Holm, R. Roman, et al. 1987. Lipofectin: A highly efficient, lipid-mediated DNA/transfection procedure. Proc. Natl. Acad. Sci. USA. 84:7413-7417.

Felici, F., L. Castagnoli, A. Musacchio, R. Jappelli, et al. 1991. Selection of antibody ligands from a large library of oligopeptides expressed on a multivalent exposition vector. J Mol Biol. 222:301-10.

Fieck, A., D. L. Wyborski, and J. M. Short. 1992. Modifications of the E. coli Lac repressor for expression in eukaryotic cells: effects of nuclear signal sequences on protein activity and nuclear accumulation. Nucleic Acids Res. 20:1785-91.

Finer, J. J., K. R. Finer, and T. Ponappa. 1999. Particle bombardment-mediated transformation. Current Topics in microbiology and immunology. 240:59-80.

Finn, P. J., N. J. Gibson, R. Fallon, A. Hamilton, et al. 1996. Synthesis and properties of DNA-PNA chimeric oligomers. Nucleic Acids Res. 24:3357-63.

Fishwild, D. M., S. L. O'Donnell, T. Bengoechea, D. V. Hudson, et al. 1996. High-avidity human IgG kappa monoclonal antibodies from a novel strain of minilocus transgenic mice [see comments]. Nat Biotechnol. 14:845-51.

Fleer, R., P. Yeh, N. Amellal, I. Maury, et al. 1991. Stable multicopy vectors for high-level secretion of recombinant human serum albumin by Kluyveromyces yeasts. Biotechnology (NY). 9:968-75.

Fodor, S. P., R. P. Rava, X. C. Huang, A. C. Pease, et al. 1993. Multiplexed biochemical assays with biological chips. Nature. 364:555-6.

Fromm, M., L. P. Taylor, and V. Walbot. 1985. Expression of genes transferred into monocot and dicot plant cells by electroporation. Proc. Natl. Acad. Sci. USA. 82:5824-5828.

Fujiki, Y. 2000. Peroxisome biogenesis and peroxisome biogenesis disorders. FEBS Lett. 476:42-6.

Fujita, T., H. Shubiya, T. Ohashi, K. Yamanishi, et al. 1986. Regulation of human interleukin-2 gene: Functional DNA sequences in the 5′ flanking region for the gene expression in activated T lymphocytes. Cell. 46:401-407.

Gabizon, A., R. Shiota, and D. Papahadjopoulos. 1989. Pharmacokinetics and tissue distribution of doxorubicin encapsulated in stable liposomes with long circulation times. J Natl Cancer Inst. 81:1484-8.

Gallagher, S. R. 1992. GUS protocols: Using the GUS gene as a reporter of gene expression. Academic Press, San Diego, Calif.

Gallop, M. A., R. W. Barrett, W. J. Dower, S. P. Fodor, et al. 1994. Applications of combinatorial technologies to drug discovery. 1. Background and peptide combinatorial libraries. J Med Chem. 37:1233-51.

Gasparini, P., A. Bonizzato, M. Dognini, and P. F. Pignatti. 1992. Restriction site generating-polymerase chain reaction (RG-PCR) for the probeless detection of hidden genetic variation: application to the study of some common cystic fibrosis mutations. Mol Cell Probes. 6:1-7.

Gautier, C., F. Morvan, B. Rayner, T. Huynh-Dinh, et al. 1987. Alpha-DNA. IV: Alpha-anomeric and beta-anomeric tetrathymidylates covalently linked to intercalating oxazolopyridocarbazole. Synthesis, physicochemical properties and poly (rA) binding. Nucleic Acids Res. 15:6625-41.

Gennaro, A. R. 2000. Remington: The science and practice of pharmacy. Lippincott, Williams & Wilkins, Philadelphia, Pa.

Gibbs, R. A., P. N. Nguyen, and C. T. Caskey. 1989. Detection of single DNA base differences by competitive oligonucleotide priming. Nucleic Acids Res. 17:2437-48.

Gietz, R. D., R. A. Woods, P. Manivasakam, and R. H. Schiestl. 1998. Growth and transformation of Saccharomyces cerevisiae. In Cells: A laboratory manual. Vol. I. D. Spector, R. Goldman, and L. Leinwand, editors. Cold Spring Harbor Press, Cold Spring Harbor, N.Y.

Goding, J. W. 1996. Monoclonal antibodies: Principles and Practice. Academic Press, San Diego. 492 pp.

Gorman, C. M., L. F. Moffat, and B. H. Howard. 1982. Recombinant genomes which express chloramphenicol acetyltransferase in mammalian cells. Mol. Cell. Biol. 2:1044-1051.

Graham, F. L., and A. J. van der Eb. 1973. A new technique for the assay of infectivity of human adenovirus 5 DNA. Virology. 52:456-.

Griffin, H. G., and A. M. Griffin. 1993. DNA sequencing. Recent innovations and future trends. Appl Biochem Biotechnol. 38:147-59.

Grompe, M., D. M. Muzny, and C. T. Caskey. 1989. Scanning detection of mutations in human ornithine transcarbamoylase by chemical mismatch cleavage. Proc Natl Acad Sci USA.86:5888-92.

Gruber, M., B. A. Schodin, E. R. Wilson, and D. M. Kranz. 1994. Efficient tumor cell lysis mediated by a bispecific single chain antibody expressed in Escherichia coli. J Immunol. 152:5368-74.

Guan, X. M., H. Yu, and L. H. Van der Ploeg. 1998. Evidence of altered hypothalamic pro-opiomelanocortin/neuropeptide Y mRNA expression in tubby mice. Brain Res Mol Brain Res. 59:273-9.

Guatelli, J. C., K. M. Whitfield, D. Y. Kwoh, K. J. Barringer, et al. 1990. Isothermal, in vitro amplification of nucleic acids by a multienzyme reaction modeled after retroviral replication. Proc Natl Acad Sci USA. 87:1874-8.

Hanahan, D. 1983. Studies on transformation of Escherichia coli with plasmids. J. Mol. Biol. 166:557-580.

Hansen, G., and M.-D. Chilton. 1999. Lessons in gene transfer to plants by a gifted microbe. Curr. Top. Microbiol. Immunol. 240:21-57.

Hansen, G., and M. S. Wright. 1999. Recent advances in the transformation of plants. Trends Plant Sci. 4:226-231.

Harlow, E., and D. Lane. 1988. Antibodies: A laboratory manual. Cold Spring Harbor Laboratory Press, Cold Spring Harbor. 726 pp.

Harlow, E., and D. Lane. 1999. Using antibodies: A laboratory manual. Cold Spring Harbor Laboratory Press, Cold Spring Harbor, N.Y.

Haseloff, J., and W. L. Gerlach. 1988. Simple RNA enzymes with new and highly specific endoribonuclease activities. Nature. 334:585-91.

Hayashi, K. 1992. PCR-SSCP: A method for detection of mutations. Genetic and Analytical Techniques Applications. 9:73-79.

Helene, C. 1991. The anti-gene strategy: control of gene expression by triplex-forming-oligonucleotides. Anticancer Drug Des. 6:569-84.

Helene, C., N. T. Thuong, and A. Harel-Bellan. 1992. Control of gene expression by triple helix-forming oligonucleotides. The antigene strategy. Ann N Y Acad Sci. 660:27-36.

Heymsfield, S. B., A. S. Greenberg, K. Fujioka, R. M. Dixon, et al. 1999. Recombinant leptin for weight loss in obese and lean adults: a randomized, controlled, dose-escalation trial. Jama. 282:1568-75.

Hill, J. O., and J. C. Peters. 1998. Environmental contributions to the obesity epidemic. Science. 280:1371-4.

Hinnen, A., J. B. Hicks, and G. R. Fink. 1978. Transformation of yeast. Proc. Natl. Acad. Sci. USA. 75:1929-1933.

Hoffman, F. 1996. Laser microbeams for the manipulation of plant cells and subcellular structures. Plant Sci. 113:1-11.

Hogan, B., Beddington, R., Costantini, F., Lacy, E. 1994. Manipulating the Mouse Embryo: A Laboratory Manual. Cold Spring Harbor Laboratory Press. 500 pp.

Holliger, P., T. Prospero, and G. Winter. 1993. “Diabodies”: small bivalent and bispecific antibody fragments. Proc Natl Acad Sci USA. 90:6444-8.

Hoogenboom, H. R., A. D. Griffiths, K. S. Johnson, D. J. Chiswell, et al. 1991. Multi-subunit proteins on the surface of filamentous phage: methodologies for displaying antibody (Fab) heavy and light chains. Nucleic Acids Res. 19:4133-7.

Houghten, R. A., J. R. Appel, S. E. Blondelle, J. H. Cuervo, et al. 1992. The use of synthetic peptide combinatorial libraries for the identification of bioactive peptides. Biotechniques. 13:412-21.

Hsu, I. C., Q. Yang, M. W. Kahng, and J. F. Xu. 1994. Detection of DNA point mutations with DNA mismatch repair enzymes. Carcinogenesis. 15:1657-62.

Hwang, K. J., K. F. Luk, and P. L. Beaumier. 1980. Hepatic uptake and degradation of unilamellar sphingomyelin/cholesterol liposomes: a kinetic study. Proc Natl Acad Sci USA. 77:4030-4.

Hyrup, B., and P. E. Nielsen. 1996. Peptide nucleic acids (PNA): synthesis, properties and potential applications. Bioorg Med Chem. 4:5-23.

Infante, J. P., and V. A. Huszagh. 2001. Zellweger syndrome knockout mouse models challenge putative peroxisomal beta-oxidation involvement in docosahexaenoic acid (22:6n-3) biosynthesis. Mol Genet Metab. 72:1-7.

Inoue, H., Y. Hayase, A. Imura, S. Iwai, et al. 1987a. Synthesis and hybridization studies on two complementary nona(2′-O-methyl)ribonucleotides. Nucleic Acids Res. 15:6131-48.

Inoue, H., Y. Hayase, S. Iwai, and E. Ohtsuka. 1987b. Sequence-dependent hydrolysis of RNA using modified oligonucleotide splints and RNase H. FEBS Lett. 215:327-30.

Ishiura, M., S. Hirose, T. Uchida, Y. Hamada, et al. 1982. Phage particle-mediated gene transfer to cultured mammalian cells. Molecular and Cellular Biology. 2:607-616.

Ito, H., Y. Fukuda, K. Murata, and A. Kimura. 1983. Transformation of intact yeast cells treated with alkali cations. J. Bacteriol. 153:163-168.

Iwabuchi, K., B. Li, P. Bartel, and S. Fields. 1993. Use of the two-hybrid system to identify the domain of p53 involved in oligomerization. Oncogene. 8:1693-6.

Jayasena, S. D. 1999. Aptamers: an emerging class of molecules that rival antibodies in diagnostics. Clin Chem. 45:1628-50.

Jones, P. T., P. H. Dear, J. Foote, M. S. Neuberger, et al. 1986. Replacing the complementarity-determining regions in a human antibody with those from a mouse. Nature. 321:522-5.

Kaufman, R. J. 1990. Vectors used for expression in mammalian cells. Methods Enzymol. 185:487-511.

Kaufman, R. J., P. Murtha, D. E. Ingolia, C.-Y. Yeung, et al. 1986. Selection and amplification of heterologous genes encoding adenosine deaminase in mammalian cells. Proc. Natl. Acad. Sci. USA. 83:3136-3140.

Kawai, S., and M. Nishizawa. 1984. New procedure for DNA transfection with polycation and dimethyl sulfoxide. Mol. Cell. Biol. 4:1172.

Keen, J., D. Lester, C. Inglehearn, A. Curtis, et al. 1991. Rapid detection of single base mismatches as heteroduplexes on Hydrolink gels. Trends Genet. 7:5.

Kelly, J. M., and M. J. Hynes. 1985. Transformation of Aspergillus niger by the amdS gene of Aspergillus nidulans. Embo J. 4:475-9.

Kersten, S. 2001. Mechanisms of nutritional and hormonal regulation of lipogenesis. EMBO Rep. 2:282-6.

Kostelny, S. A., M. S. Cole, and J. Y. Tso. 1992. Formation of a bispecific antibody by the use of leucine zippers. J Immunol. 148:1547-53.

Koster, H. WO94/16101. DNA SEQUENCING BY MASS SPECTROMETRY. 1994.

Kozal, M. J., N. Shah, N. Shen, R. Yang, et al. 1996. Extensive polymorphisms observed in HIV-1 clade B protease gene using high-density oligonucleotide arrays. Nat Med. 2:753-9.

Kozbor, D., P. Tripputi, J. C. Roder, and C. M. Croce. 1984. A human hybrid myeloma for production of human monoclonal antibodies. J Immunol. 133:3001-5.

Kriegler, M. 1990. Gene transfer and expression: A laboratory manual. Stockton Press, New York. 242 pp.

Kucherlapati, R. S., B. H. Koller, and O. Smithies. WO 91/01140. HOMOLOGOUS RECOMBINATION FOR UNIVERSAL DONOR CELLS AND CHIMERIC MAMMALIAN HOSTS. 1991.

Kwoh, D. Y., G. R. Davis, K. M. Whitfield, H. L. Chappelle, et al. 1989. Transcription-based amplification system and detection of amplified human immunodeficiency virus type 1 with a bead-based sandwich hybridization format. Proc Natl Acad Sci USA. 86:1173-7.

Ladner, R. C., S. K. Guterman, B. L. Roberts, W. Markland, et al. U.S. Pat. No. 5,223,409. Directed evolution of novel binding proteins. 1993.

Lakso, M., B. Sauer, B. Mosinger, E. J. Lee, et al. 1992. Targeted oncogene activation by site-specific recombination in transgenic mice. Proc Natl Acad Sci USA. 89:6232-6.

Lam, K. S. 1997. Application of combinatorial library methods in cancer research and drug discovery. Anticancer Drug Design. 12:145-167.

Lam, K. S., S. E. Salmon, E. M. Hersh, V. J. Hruby, et al. 1991. General method for rapid synthesis of multicomponent peptide mixtures. Nature. 354:82-84.

Landegren, U., R. Kaiser, J. Sanders, and L. Hood. 1988. A ligase-mediated gene detection technique. Science. 241:1077-80.

Le Mouellic, H., and P. Brullet. WO 90/11354. Process for the specific replacement of a copy of a gene present in the receiver genome via the integration of a gene. 1990.

Leder, P., and T. A. Stewart. U.S. Pat. No. 4,736,866. Transgenic non-human animals. 1988.

Leduc, N., and e. al. 1996. Isolated maize zygotes mimic in vivo embryogenic development and express microinjected genes when cultured in vitro. Dev. Biol. 10:190-203.

Lee, J. S., D. A. Johnson, and A. R. Morgan. 1979. Complexes formed by (pyrimidine)n.(purine)n DNAs on lowering the pH are three-stranded. Nucleic Acids Res. 6:3073-91.

Lee, V. H. L. 1990. Peptide and protein drug delivery. Marcel Dekker, New York, N.Y. Lefebvre, O., M. P. Chenard, R. Masson, J. Linares, et al. 1996. Gastric mucosa abnormalities and tumorigenesis in mice lacking the pS2 trefoil protein. Science. 274:259-62.

Lemaitre, M., B. Bayard, and B. Lebleu. 1987. Specific antiviral activity of a poly(L-lysine)-conjugated oligodeoxyribonucleotide sequence complementary to vesicular stomatitis virus N protein mRNA initiation site. Proc Natl Acad Sci USA. 84:648-52.

Lemischka, I. R., D. H. Raulet, and R. C. Mulligan. 1986. Developmental potential and dynamic behavior of hematopoietic stem cells. Cell. 45:917-927.

Letsinger, R. L., G. R. Zhang, D. K. Sun, T. Ikeuchi, et al. 1989. Cholesteryl-conjugated oligonucleotides: synthesis, properties, and activity as inhibitors of replication of human immunodeficiency virus in cell culture. Proc Natl Acad Sci USA. 86:6553-6.

Li, E., T. H. Bestor, and R. Jaenisch. 1992. Targeted mutation of the DNA methyltransferase gene results in embryonic lethality. Cell. 69:915-26.

Linder, M. W., R. A. Prough, and R. Valdes. 1997. Pharmacogenetics: a laboratory tool for optimizing therapeutic efficiency. Clin Chem. 43:254-66.

Littlefield, J. W. 1964. Selection of hybrids from matings of fibroblasts in vitro and their presumed recombinants. Science. 145:709-710.

Lizardi, P. M., C. E. Guerra, H. Lomeli, I. Tussie-Luna, et al. 1988. Exponential amplification of recombinant-RNA hybridization probes. Biotechnology. 6:1197-1202.

Lonberg, N., and D. Huszar. 1995. Human antibodies from transgenic mice. Int Rev Immunol. 13:65-93.

Lonberg, N., L. D. Taylor, F. A. Harding, M. Trounstine, et al. 1994. Antigen-specific human antibodies from mice comprising four distinct genetic modifications [see comments]. Nature. 368:856-9.

Lopata, M. A., D. W. Cleveland, and B. Sollner-Webb. 1984. High-level expression of a chloramphenicol acetyltransferase gene by DEAEdextran-mediated DNA traansfection couled with a dimethylsulfoxide or glycerol shock treatment. Nucleic Acids Research. 12:5707.

Luckow, V. A. 1991. Cloning and expression of heterologous genes in insect cells with baculovirus vectors. In Recombinant DNA technology and applications. A. Prokop, R. K. Bajpai, and C. Ho, editors. McGraw-Hill, New York. 97-152.

Madura, K., R. J. Dohmen, and A. Varshavsky. 1993. N-recognin/Ubc2 interactions in the N-end rule pathway. J Biol Chem. 268:12046-54.

Maher, L. J. 1992. DNA triple-helix formation: an approach to artificial gene repressors? Bioessays. 14:807-15.

Mandel, M., and A. Higa. 1970. Calcium-dependent bacteriophage DNA infection. J. Mol. biol. 53:159-162.

Marasco, W. A., W. A. Haseltine, and S. Y. Chen. 1993. Design, intracellular expression, and activity of a human anti-human immunodeficiency virus type 1 gp120 single-chain antibody. Proc Natl Acad Sci USA. 90:7889-93.

Marks, J. D., A. D. Griffiths, M. Malmqvist, T. P. Clackson, et al. 1992. By-passing immunization: building high affinity human antibodies by chain shuffling. Biotechnology (NY). 10:779-83.

Marks, J. D., H. R. Hoogenboom, T. P. Bonnert, J. McCafferty, et al. 1991. By-passing immunization. Human antibodies from V-gene libraries displayed on phage. J. Mol Biol. 222:581-97.

Martin, F. J., and D. Papahadjopoulos. 1982. Irreversible coupling of immunoglobulin fragments to preformed vesicles. An improved method for liposome targeting. J Biol Chem. 257:286-8.

Maxam, A. M., and W. Gilbert. 1977. A new method for sequencing DNA. Proc Natl Acad Sci USA. 74:560-4.

Miller, A. D., and C. Buttimore. 1986. Redesign of retrovirus packaging cell lines to avoid recombination leading to helper virus production. Mol. Cell biol. 6:2895-2902.

Miller, L. K. 1988. Baculoviruses as gene expression vectors. Annu. Rev. Microbiol. 42:177-199.

Milstein, C., and A. C. Cuello. 1983. Hybrid hybridomas and their use in immunohistochemistry. Nature. 305:537-40.

Modrich, P., S.-S. Su, K. G. Au, and R. S. Lahue. U.S. Pat. No. 5,459,039. Methods for mapping genetic mutations. 1995.

Montague, C. T., I. S. Farooqi, J. P. Whitehead, M. A. Soos, et al. 1997. Congenital leptin deficiency is associated with severe early-onset obesity in humans. Nature. 387:903-8.

Morrison, S. L., L. Wims, S. Wallick, L. Tan, et al. 1987. Genetically engineered antibody molecules and their application. Ann NY Acad Sci. 507:187-98.

Mullis, K. B. U.S. Pat. No. 4,683,202. Process for amplifying nucleic acid sequences. 1987.

Mullis, K. B., H. A. Erlish, N. Arnheim, G. T. Horn, et al. U.S. Pat. No. 4,683,195. Process for amplifying, detecting, and/or cloning nucleic acid sequences. 1987.

Munson, P. J., and D. Rodbard. 1980. Ligand: a versatile computerized approach for characterization of ligand-binding systems. Anal Biochem. 107:220-39.

Myers, R. M., Z. Larin, and T. Maniatis. 1985. Detection of single base substitutions by ribonuclease cleavage at mismatches in RNA:DNA duplexes. Science. 230:1242-6.

Nabel, E. G., and G. J. Nabel. U.S. Pat. No. 5,328,470. Treatment of diseases by site-specific instillation of cells or site-specific transformation of cells and kits therefor. 1994.

Naeve, C. W., G. A. Buck, R. L. Niece, R. T. Pon, et al. 1995. Accuracy of automated DNA sequencing: a multi-laboratory comparison of sequencing results. Biotechniques. 19:448-53.

Nakai, K., and P. Horton. 1999. PSORT: a program for detecting sorting signals in proteins and predicting their subcellular localization. Trends Biochem Sci. 24:34-6.

Nakazato, M., N. Murakami, Y. Date, M. Kojima, et al. 2001. A role for ghrelin in the central regulation of feeding. Nature. 409:194-8.

Nakazawa, H., D. English, P. L. Randell, K. Nakazawa, et al. 1994. UV and skin cancer: specific p53 gene mutation in normal skin as a biologically relevant exposure measurement. Proc Natl Acad Sci USA. 91:360-4.

Neumann, E., M. Schaefer-Ridder, Y. Wang, and P. H. Hofschneider. 1982. Gene transfer into mouse lyoma cells by electroporation in high electric fields. EMBO J. 1:841-845.

Norman, R. A., C. Bogardus, and E. Ravussin. 1995. Linkage between obesity and a marker near the tumor necrosis factor-alpha locus in Pima Indians. J Clin Invest. 96:158-62.

O'Gorman, S., D. T. Fox, and G. M. Wahl. 1991. Recombinase-mediated gene activation and site-specific integration in mammalian cells. Science. 251:1351-5.

Okano, H., J. Aruga, T. Nakagawa, C. Shiota, et al. 1991. Myelin basic protein gene and the function of antisense RNA in its repression in myelin-deficient mutant mouse. J Neurochem. 56:560-7.

O'Reilly, D. R., L. K. Miller, and V. A. Luckow. 1992. Baculovirus expression vectors. W. H. Freeman and Company, New York.

Orita, M., H. Iwahana, H. Kanazawa, K. Hayashi, et al. 1989. Detection of polymorphisms of human DNA by gel electrophoresis as single-strand conformation polymorphisms. Proc Natl Acad Sci USA. 86:2766-70.

Ou-Lee, T. M., R. Turgeon, and R. Wu. 1986. Uptake and expression of a foreign gene linked to either a plant virus or Drosophila promoter in protoplasts of rice, wheat and sorghum. Proc. Natl. Acad. Sci. USA. 83:6815-6819.

Palmer, T. D., R. A. Hock, W. R. A. osborne, and A. D. Miller. 1987. Efficient retrovirus-mediated transfer and expression of a human adenosine deaminase gene in diploid skin fibroblasts from an adenosie-deficient human. Proc. Natl. Acad. Sci. USA. 84:1055-1059.

Pardridge, W., and P. Schimmel. W089/10134. Chimeric peptides for neuropeptide delivery through the blood-brain barrier. 1989.

Pear, W., G. Nolan, M. Scott, and D. Baltimore. 1993. Production of high-titer helper-free retroviruses by transient transfection. Proc. Natl Acad. Sci. USA. 90:8392-8396.

Perry-O'Keefe, H., X. W. Yao, J. M. Coull, M. Fuchs, et al. 1996. Peptide nucleic acid pre-gel hybridization: an alternative to southern hybridization. Proc Natl Acad Sci USA. 93:14670-5.

Perusse, L., and C. Bouchard. 1999. Role of genetic factors in childhood obesity and in susceptibility to dietary variations. Ann Med. 31 Suppl 1:19-25.

Petersen, K. H., D. K. Jensen, M. Egholm, O. Buchardt, et al. 1976. A PNA-DNA linker synthesis of N-((4,4′-dimethoxytrityloxy)ehtyl)-N-(thymin-1-ylacetyl)glycine. Biorganic and Medicianl Chemistry Letters. 5:1119-1124.

Phillips, M. S., Q. Liu, H. A. Hammond, V. Dugan, et al. 1996. Leptin receptor missense mutation in the fatty Zucker rat. Nat Genet. 13:18-9.

Pi-Sunjer, F. C., and E. NHLBI Obesity Education Initiative Expert Panel on the Identification, and Treatment of Overweight and Obesity in Adults. 1998. Clinical guidelines on the identification, evaluation, and treatment of overweight and obesity in adults. In The evidence report. National Institutes of Health, Bethesda, Md. 263.

Playford, R. J., T. Marchbank, R. A. Goodlad, R. A. Chinery, et al. 1996. Transgenic mice that overexpress the human trefoil peptide pS2 have an increased resistance to intestinal damage. Proc Natl Acad Sci USA. 93:2137-42.

Potter, H. 1988. Electroporation in biology: Methods, applications, and instrumentation. Analytical Biochemistry. 174:361-373.

Potter, H., L. Weir, and P. Leder. 1984. Enhancer-dependent expression of human kappa immunoglobulin genes introduced into mouse pre-B lymphocytes by electroporation. Proc. Natl. Acad. Sci. USA. 81:7161-7165.

Presta, L. G. 1992. Antibody engineering. Curr Opin Biotechnol. 3:394-8.

Prosser, J. 1993. Detecting single-base mutations. Trends Biotechnol. 11:238-46.

Rassoulzadegan, M., B. Binetruy, and F. Cuzin. 1982. High frequency of gene transfer after fusion between bacteria and eukaryotic cells. Nature. 295:257.

Reisfeld, R. A., and S. Sell. 1985. Monoclonal antibodies and cancer therapy: Proceedings of the Roche-UCLA symposium held in Park City, Utah, Jan. 26-Feb. 2, 1985. Alan R. Liss, New York. 609 pp.

Report, A. 1997. Position of the American Dietetic Association: weight management. J Am Diet Assoc. 97:71-4.

Rhodes, C. A., D. A. Pierce, I. J. Mettler, D. Mascarenhas, et al. 1988. Genetically transformed maize plants from protoplasts. Science. 240:204-207.

Riechmann, L., M. Clark, H. Waldmann, and G. Winter. 1988. Reshaping human antibodies for therapy. Nature. 332:323-7.

Rose, J. K., L. Buonocore, and M. Whitt. 1991. A new cationic liposome reagent mediating nearly quantitative transfection of animal cells. BioTechniques. 10:520-525.

Rossiter, B. J., and C. T. Caskey. 1990. Molecular scanning methods of mutation detection. J Biol Chem. 265:12753-6.

Saifer, M., R. Somack, and L. D. Williams. U.S. Pat. No. 5,283,317. Intermediates for conjugation of polypeptides with high molecular weight polyalkylene glycols. 1994.

Saiki, R. K., T. L. Bugawan, G. T. Horn, K. B. Mullis, et al. 1986. Analysis of enzymatically amplified beta-globin and HLA-DQ alpha DNA with allele-specific oligonucleotide probes. Nature. 324:163-6.

Saiki, R. K., P. S. Walsh, C. H. Levenson, and H. A. Erlich. 1989. Genetic analysis of amplified DNA with immobilized sequence-specific oligonucleotide probes. Proc Natl Acad Sci USA. 86:6230-4.

Sakurai, T., A. Amemiya, M. Ishii, I. Matsuzaki, et al. 1998a. Orexins and orexin receptors: a family of hypothalamic neuropeptides and G protein-coupled receptors that regulate feeding behavior. Cell. 92:573-85.

Sakurai, T., A. Amemiya, M. Ishii, I. Matsuzaki, et al. 1998b. Orexins and orexin receptors: a family of hypothalamic neuropeptides and G protein-coupled receptors that regulate feeding behavior. Cell. 92:1 page following 696.

Saleeba, J. A., and R. G. Cotton. 1993. Chemical cleavage of mismatch to detect mutations. Methods Enzymol. 217:286-95.

Sambrook, J. 1989. Molecular cloning: a laboratory manual. Cold Spring Harbor Laboratory, Cold Spring Harbor.

Sandri-Goldin, R. M., A. L. Goldin, J. C. Glorioso, and M. Levine. 1981. High-frequency transfer of cloned herpes simjplex virus type I sequences to mammalian cells by protoplast fusion. Mol. Cell. Biol. 1:7453-752.

Sanger, F., S. Nicklen, and A. R. Coulson. 1977. DNA sequencing with chain-terminating inhibitors. Proc Natl Acad Sci USA. 74:5463-7.

Saunders, J. A., B. F. Matthews, and P. D. Miller. 1989. Plant gene transfer using electrofusion and electroporation. In Electroporation and electrofusion in cell biology. E. Neumann, A. E. Sowers, and C. A. Jordan, editors. Plenum Press, New York. 343-354.

Schade, R., C. Staak, C. Hendriksen, M. Erhard, et al. 1996. The production of avian (egg yold) antibodies: IgY. The report and recommendations of ECVAM workshop. Alternatives to Laboratory Animals (ATLA). 24:925-934.

Schaffner, W. 1980. Direct transfer of cloned genes from bacteria to mammalian cells. Proc. Natl. Acad. Sci. USA. 77:2163.

Schook, L. B. 1987. Monoclonal antibody production techniques and applications. Marcel Dekker, Inc., New York. 336 pp.

Schrauwen, P., K. Walder, and E. Ravussin. 1999. Human uncoupling proteins and obesity. Obes Res. 7:97-105.

Scott, J. K., and G. P. Smith. 1990. Searching for peptide ligands with an epitope library. Science. 249:386-90.

Selden, R. F., K. Burke-Howie, M. E. Rowe, H. M. Goodman, et al. 1986. Human growth hormone as a reporter gene in regulation studies employing transient gene expression. Molecular and Cellular Biololgy. 6:3173-3179.

Shalaby, M. R., H. M. Shepard, L. Presta, M. L. Rodrigues, et al. 1992. Development of humanized bispecific antibodies reactive with cytotoxic lymphocytes and tumor cells overexpressing the HER2 protooncogene. J Exp Med. 175:217-25.

Shigekawa, K., and W. J. Dower. 1988. Electroporation of eukaryotes and prokaryotes: A general approach to the introduction of macomolecules into cells. BioTechniques. 6:742-751.

Shillito, R. 1999. Methods of genetic transformations: Electroporation and polyethylene glycol treatment. In Molecular improvement of cereal crop. I. Vasil, editor. Kluwer, Dordrecht, The Netherlands. 9-20.

Shilo, B. Z., and R. A. Weinberg. 1981. DNA sequences homologous to vertebrate oncogenes are conserved in Drosophila melanogaster. Proc Natl Acad Sci USA. 78:6789-92.

Shimkets, R. A., D. G. Lowe, J. T. Tai, P. Sehl, et al. 1999. Gene expression analysis by transcript profiling coupled to a gene database query. Nat Biotechnol. 17:798-803.

Shopes, B. 1992. A genetically engineered human IgG mutant with enhanced cytolytic activity. J Immunol. 148:2918-22.

Simonsen, C. C., and A. D. Levinson. 1983. Isolation and expression of an altered mouse dihydrofolate reductase cDNA. Proc. Natl. Acad. Sci. USA. 80:2495-2499.

Sjarif, D. R., J. K. Ploos van Amstel, M. Duran, F. A. Beemer, et al. 2000. Isolated and contiguous glycerol kinase gene disorders: a review. J Inherit Metab Dis. 23:529-47.

Smulson, M. E., B. Kishor, and H. Konrad. U.S. Pat. No. 5,272,057. Method of detecting a predisposition to cancer by the use of restriction fragment length polymorphism of the gene for human poly (ADP-ribose) polymerase. 1993.

Southern, P. J., and P. Berg. 1982. Transformation of mammalian cells to antibiotic resistanced with a bacterial gene under control of the SV40 early region promoter. J. Mol. Appl. Gen. 1:327-341.

Spiegelman, B. M., and J. S. Flier. 1996. Adipogenesis and obesity: rounding out the big picture. Cell. 87:377-89.

Sreekrishna, K., R. H. Potenz, J. A. Cruze, W. R. McCombie, et al. 1988. High level expression of heterologous proteins in methylotrophic yeast Pichia pastoris. J Basic Microbiol. 28:265-78.

Stein, C. A., and J. S. Cohen. 1988. Oligodeoxynucleotides as inhibitors of gene expression: a review. Cancer Res. 48:2659-68.

Stevenson, G. T., A. Pindar, and C. J. Slade. 1989. A chimeric antibody with dual Fc regions (bisFabFc) prepared by manipulations at the IgG hinge. Anticancer Drug Des. 3:219-30.

Strosberg, A. D. 1997. Structure and function of the beta 3-adrenergic receptor. Annu Rev Pharmacol Toxicol. 37:421-50.

Suresh, M. R., A. C. Cuello, and C. Milstein. 1986. Bispecific monoclonal antibodies from hybrid hybridomas. Methods Enzymol. 121:210-28.

Thomas, K. R., and M. R. Capecchi. 1987. Site-directed mutagenesis by gene targeting in mouse embryo-derived stem cells. Cell. 51:503-12.

Thompson, J., D. Higgins, and T. Gibson. 1994. CLUSTAL W: Improving the sensitivity of progressive multiple sequence alignment through sequence weighting, positions-specific gap penalties and weight matrix choice. Nucl. Ac. Res. 22:4673-4680.

Thompson, J. A., and e. al. 1995. Maize transformation utilizing silicon carbide whiskers: A review. Euphytica. 85:75-80.

Tilburn, J., C. Scazzocchio, G. G. Taylor, J. H. Zabicky-Zissman, et al. 1983. Transformation by integration in Aspergillus nidulans. Gene. 26:205-21.

Touraev, A., and e. al. 1997. Plant male germ line transformation. Plant J. 12:949-956.

Traunecker, A., F. Oliveri, and K. Kadjalainen. 1991. Myeloma based expression system for production of large mammalian proteins. Trends Biotechnol. 9:109-13.

Trick, H. N., and e. al. 1997. Recent advances in soybean transformation. Plant Tissue Cult. Biotechnol. 3:9-26.

Tuerk, C., and L. Gold. 1990. Systematic evolution of ligands by exponential enrichment: RNA ligands to bacteriophage T4 DNA polymerase. Science. 249:505-10.

Turner, D. L., E. Y. Snyder, and C. L. Cepko. 1990. Lineage-independent determinationh of cell type in the embryonic mouse retina. Neuron. 4:833-845.

Tutt, A., G. T. Stevenson, and M. J. Glennie. 1991. Trispecific F(ab′)3 derivatives that use cooperative signaling via the TCR/CD3 complex and CD2 to activate and redirect resting cytotoxic T cells. J Immunol. 147:60-9.

van der Krol, A. R., J. N. Mol, and A. R. Stuitje. 1988a. Modulation of eukaryotic gene expression by complementary RNA or DNA sequences. Biotechniques. 6:958-76.

van der Krol, A. R., J. N. Mol, and A. R. Stuitje. 1988b. Modulation of eukaryotic gene expression by complementary RNA or DNA sequences. Biotechniques. 6:958-76.

Verhoeyen, M., C. Milstein, and G. Winter. 1988. Reshaping human antibodies: grafting an antilysozyme activity. Science. 239:1534-6.

Vitetta, E. S., R. J. Fulton, R. D. May, M. Till, et al. 1987. Redesigning nature's poisons to create anti-tumor reagents. Science. 238:1098-104.

Wagner, T. E., and P. C. Hoppe. U.S. Pat. No. 4,873,191. Genetic transformation of zygotes. 1989.

Weigle, D. S., and J. L. Kuijper. 1996. Obesity genes and the regulation of body fat content. Bioessays. 18:867-74.

Wells, J. A., M. Vasser, and D. B. Powers. 1985. Cassette mutagenesis: an efficient method for generation of multiple mutations at defined sites. Gene. 34:315-23.

Whitt, M. A., L. Buonocore, J. K. Rose, V. Ciccarone, et al. 1990. TransfectACE reagent promotes transient transfection frequencies greater than 90%. Focus. 13:8-12.

Wigler, M., A. Pellicer, S. Silversttein, and R. Axel. 1978. Biochemical transfer of single-copy eucaryotic genes using total cellular DNA as donor. Cell. 14:725.

Wikberg, J. E., R. Muceniece, I. Mandrika, P. Prusis, et al. 2000. New aspects on the melanocortins and their receptors. Pharmacol Res. 42:393-420.

Williams, D. A., I. R. Lemischka, D. G. Nathan, and R. C. Mulligan. 1984. Introduction of a new genetic material into pluripotent haematopoietic stem cells of the mouse. Nature. 310:476-480.

Wilmut, I., A. E. Schnieke, J. McWhir, A. J. Kind, et al. 1997. Viable offspring derived from fetal and adult mammalian cells. Nature. 385:810-3.

Wolff, E. A., G. J. Schreiber, W. L. Cosand, and H. V. Raff. 1993. Monoclonal antibody homodimers: enhanced antitumor activity in nude mice. Cancer Res. 53:2560-5.

Wong, T. K., and E. Neumann. 1982. Electric field mediated gene transfer. Biochemical and Biophysical Research Communications. 107:584-587.

Wong, W. M., R. Poulsom, and N. A. Wright. 1999. Trefoil peptides. Gut. 44:890-5.

Wright, N. A., W. Hoffmann, W. R. Otto, M. C. Rio, et al. 1997. Rolling in the clover: trefoil factor family (TFF)-domain peptides, cell migration and cancer. FEBS Lett. 408:121-3.

Wyborski, D. L., L. C. DuCoeur, and J. M. Short. 1996. Parameters affecting the use of the lac repressor system in eukaryotic cells and transgenic animals. Environ Mol Mutagen. 28:447-58.

Wyborski, D. L., and J. M. Short. 1991. Analysis of inducers of the E. coli lac repressor system in mammalian cells and whole animals. Nucleic Acids Res. 19:4647-53.

Yaswen, L., N. Diehl, M. B. Brennan, and U. Hochgeschwender. 1999. Obesity in the mouse model of pro-opiomelanocortin deficiency responds to peripheral melanocortin. Nat Med. 5:1066-70.

Yelton, M. M., J. E. Hamer, and W. E. Timberlake. 1984. Transformation of Aspergillus nidulans by using a trpC plasmid. Proc Natl Acad Sci USA. 81:1470-4.

Zervos, A. S., J. Gyuris, and R. Brent. 1993. Mxi1, a protein that specifically interacts with Max to bind Myc-Max recognition sites. Cell. 72:223-32.

Zhou, G., and e. al. 1983. Introduction of exogenous DNA into cotton embryos. Methods Enzymol. 101:433-481.

Zoller, M. J., and M. Smith. 1987. Oligonucleotide-directed mutagenesis: a simple method using two oligonucleotide primers and a single-stranded DNA template. Methods Enzymol. 154:329-50.

Zon, G. 1988. Oligonucleotide analogues as potential chemotherapeutic agents. Pharm Res. 5:539-49.

Zuckermann, R. N., E. J. Martin, D. C. Spellmeyer, G. B. Stauber, et al. 1994. Discovery of nanomolar ligands for 7-transmembrane G-protein-coupled receptors from a diverse N-(substituted)glycine peptoid library. J Med Chem. 37:2678-85.

1. An isolated polypeptide comprising an amino acid sequence having at least 80% sequence identity to the sequence of SEQ ID NOS: 2, 7 or 12.

2. The polypeptide of claim 1, wherein said polypeptide is an active GLK, PMAP or TFF1 polypeptide.

3. The polypeptide of claim 2, wherein said amino acid sequence has at least 90% sequence identity to the sequence of SEQ ID NOS: 2, 7 or 12.

4. The polypeptide of claim 2, wherein said amino acid sequence has at least 98% sequence identity to the sequence of SEQ ID NOS: 2, 7 or 12.

5. An isolated polynucleotide encoding the polypeptide of any one of claims 1-4, or a complement of said polynucleotide.

6. An isolated polynucleotide comprising a nucleotide sequence having at least 80% sequence identity to the sequence of SEQ ID NOS: 1, 6 or 11, or a complement of said polynucleotide.

7. The polynucleotide of claim 6, wherein said nucleotide sequence has at least 90% sequence identity to the sequence of SEQ ID NOS: 1, 6 or 11, or a complement of said polynucleotide.

8. The polynucleotide of claim 6, wherein said nucleotide sequence has at least 98% sequence identity to the sequence of SEQ ID NOS: 1, 6 or 11, or a complement of said polynucleotide.

9. An antibody that specifically binds to the polypeptide of any one of claims 1-4.

10. A method of treating metabolic disorders comprising modulating the activity of GLK, PMAP or TFF1.

11. The method of claim 10 wherein said modulating activity of GLK, PMAP or TFF1 comprises decreasing the activity of GLK, PMAP or TFF1.

12. The method of claim 11, wherein said decreasing activity comprises decreasing the expression of a GLK, PMAP or TFF1.

13. The method of claim 12, wherein said decreasing expression comprises transforming a cell to express a polynucleotide anti-sense to at least a portion of an endogenous polynucleotide encoding a GLK, PMAP or TFF1.

14. The method of claim 12, wherein said decreasing activity comprises transforming a cell to express an aptamer to GLK, PMAP or TFF1.

15. The method of claim 12, wherein said decreasing activity comprises introducing into a cell an aptamer to GLK, PMAP or TFF1.

16. The method of claim 12, wherein said decreasing activity comprises administering to a cell an antibody that selectively binds GLK, PMAP or TFF1.

17. The method of claim 12, wherein said decreasing activity comprises disrupting a GLK, PMAP or TFF1 gene.

18. The method of claim 12, wherein said metabolic disorder is cachexia.

19. The method of claim 10 wherein said modulating activity of GLK, PMAP or TFF1 comprises increasing the activity of GLK, PMAP or TFF1.

20. The method of claim 19, wherein said increasing activity comprises increasing the expression of GLK, PMAP or TFF1.

21. The method of claim 20, wherein said increasing expression of GLK, PMAP or TFF1 comprises transforming a cell with a GLK, PMAP or TFF1 polynucleotide.

22. The method claim 20, wherein said increasing activity comprises administering to a cell an antibody that selectively binds GLK, PMAP or TFF1.

23. The method of claim 10, wherein said modulation comprises controlling GLK, PMAP or TFF1 gene expression with an exogenous promoter.

24. The method of claim 23, wherein said controlling comprises operably-linking the promoter to an endogenous GLK, PMAP or TFF1 gene.

25. The method of claim 23, wherein said controlling comprises transforming a cell with a GLK, PMAP or TFF1 gene operably-linked to a promoter.

26. The method of any of claims 23-25, wherein said promoter is an inducible promoter.

27. The method of any of claims 23-26, wherein said metabolic disorder is obesity or diabetes.

28. A method of detecting a metabolic disorder, or a disorder associated with changes in GLK, PMAP or TFF1 gene expression, comprising:
detecting a change in expression or activity of GLK, PMAP or TFF1.

29. The method of claim 27 or 28, wherein said metabolic disorder is associated with an up regulation of GLK, PMAP or TFF1 activity.

30. The method of claim 29, wherein said metabolic disorder is cachexia.

31. The method of claim 27 or 28, wherein said metabolic disorder is associated with a down regulation of GLK, PMAP or TFF1 activity.

32. The method of claim 31, wherein said metabolic disorder is obesity or diabetes.

33. A method for determining whether a compound up-regulates or down-regulates the transcription of a GLK, PMAP or TFF1 gene, comprising:
contacting said compound with a composition comprising a RNA polymerase and said gene and measuring the amount of GLK, PMAP or TFF1 gene transcription.

34. The method of claim 33, wherein said composition is in a cell.

35. A method for determining whether a compound up-regulates or down-regulates the translation of a GLK, PMAP or TFF1 gene, comprising:
contacting said compound with a composition comprising a ribosome and a polynucleotide corresponding to a mRNA of said gene and measuring the amount of GLK, PMAP or TFF1 gene translation.

36. The method of claim 35, wherein said composition is in a cell.

37. A vector, comprising the polynucleotide of any one of claims 5-8.

38. A cell, comprising the vector of claim 37.

39. A transgenic non-human animal, having a disrupted GLK or PMAP gene.

40. The transgenic non-human animal of claim 39, wherein the non-human animal is a mouse.

41. A transgenic non-human animal, comprising an exogenous polynucleotide having at least 80% sequence identity to the sequence of SEQ ID NOS: 1, 6 or 11, or a complement of said polynucleotide.

42. The transgenic non-human animal of claim 41, wherein said exogenous polynucleotide has at least 90% sequence identity to the sequence of SEQ ID NOS: 1, 6 or 11, or a complement of said polynucleotide.

43. The transgenic non-human animal of claim 41, wherein said exogenous polynucleotide has at least 98% sequence identity to the sequence of SEQ ID NOS: 1, 6 or 11, or a complement of said polynucleotide.

44. A method of screening a sample for a GLK, PMAP or TFF1 gene mutation, comprising:
comparing a GLK, PMAP or TFF1 nucleotide sequence in the sample with SEQ ID NOS: 2, 7 or 12.

45. A method to measuring GLK, PMAP or TFF1 agonist or antagonist acitivity of a compound comprising:
contacting the compound with a composition comprising a polypeptide having at least 80% sequence identity to the sequence of SEQ ID NOs: 2, 7 or 12 and,
determining if GLK, PMAP or TFF1 activity is changed.

46. The method of claim 45, wherein said GLK, PMAP or TFF1 is in a cell.
